SEQUENCE LISTING (21 sequences)

SEQ ID NO: 1 - DNA, 23 bp, Artificial sequence
  siRNA probe
aannnnnnnnnnnnnnnnnnnaa

SEQ ID NO: 2 - DNA, 23 bp, Artificial sequence
  siRNA probe
aannnnnnnnnnnnnnnnnnnnn

SEQ ID NO: 3 - protein, 7 aa, Artificial sequence
  R7 cellular delivery peptide
RRRRRRR

SEQ ID NO: 4 - protein, 9 aa, Artificial sequence
  R9 cellular delivery peptide
RRRRRRRRR

SEQ ID NO: 5 - protein, 11 aa, Artificial sequence
  R11 cellular delivery peptide
RRRRRRRRRRR

SEQ ID NO: 6 - protein, 9 aa, Artificial sequence
  RRG cellular delivery peptide
RRGRGRGRR

SEQ ID NO: 7 - protein, 9 aa, Artificial sequence
  RRQ cellular delivery peptide
RRQRQRGRR

SEQ ID NO: 8 - protein, 9 aa, Artificial sequence
  RG9 cellular delivery peptide
RRRRGRRRR

SEQ ID NO: 9 - protein, 7 aa, Artificial sequence
  K7 cellular delivery peptide
KKKKKKK

SEQ ID NO: 10 - protein, 9 aa, Artificial sequence
  K9 cellular delivery peptide
KKKKKKKKK

SEQ ID NO: 11 - protein, 11 aa, Artificial sequence
  K11 cellular delivery peptide
KKKKKKKKKKK

SEQ ID NO: 12 - protein, 11 aa, Artificial sequence
  PTD3 cellular delivery peptide
YARKARRQARR

SEQ ID NO: 13 - protein, 11 aa, Artificial sequence
  PTDA cellular delivery peptide
YAAKAAAQAAA

SEQ ID NO: 14 - protein, 17 aa, Artificial sequence
  Ant(42-58) cellular delivery peptide
ERQIKIFFQNRRMKFKK

SEQ ID NO: 15 - protein, 17 aa, Artificial sequence
  AntFF cellular delivery peptide
ERQIKIWFQNRRMKWKK

SEQ ID NO: 16 - DNA, 20 bp, Artificial sequence
  FITC-labeled oligonucleotide
tcccgcgcacttgatgcatt

SEQ ID NO: 17 - RNA, 19 nt, Artificial sequence
  siRNA directed against nt 153-173 of p. pyralis luciferase
cguacgcggaauacuucga

SEQ ID NO: 18 - RNA, 19 nt, Artificial sequence
  siRNA directed against GFP
cacuugucacuacuuucuc

SEQ ID NO: 19 - protein, 16 aa, artificial sequence
  Ant(43-58) cellular delivery peptide
RQIKIFFQNRRMKFKK

SEQ ID NO: 20 - protein, 16 aa, Artificial sequence
  AntFF(43-58) cellular delivery peptide
RQIKIWFQNRRMKWKK

SEQ ID NO: 21 - protein, 20 aa, Artificial sequence
  Ant(41-60) cellular delivery peptide
TERQIKIFFQNRRMKFKKKE

(19) United States
(12) Patent Application Publication
(10) Pub. No.: US 2004/0176282 A1
(43) Pub. Date: Sep. 9, 2004

(54) Cellular delivery and activation of polypeptide-nucleic acid complexes

(76) Inventors: Brian Dalby, Carlsbad, CA (US); Robert P. Bennett, Encinitas, CA (US)

Correspondence Address: GREENLEE WINNER AND SULLIVAN P C, 5370 MANHATTAN CIRCLE, SUITE 201, BOULDER, CO, 80303, US

(21) Appl. No.: 10/755,082

(22) Filed: Jan. 9, 2004

Related U.S. Application Data
(60) Provisional application No. 60/438,778, filed on Jan. 9, 2003.

(51) Int. Cl. A61K 48/00; A61K 38/16

(52) U.S. Cl. 514/8; 514/12

(57) ABSTRACT

In general, the present invention provides compositions for the cellular delivery of nucleic acids, polypeptides and/or flourophores, molecular complexes comprising fluorescent molecules or moieties, nucleic acids and polypeptides, and methods of making and using such compositions. Light-activated dispersal of the complexes leads to the intracellular release of one or more nucleic acids and/or peptides from the compositions or complexes. The biological activities of nucleic acids, polypeptides and flourophores may be repressed within the complexes, and these activities are restored upon release from the complexes.

### CROSS-REFERENCE TO RELATED APPLICATIONS

This application claims priority under 35 U.S.C. §119(e) to U.S. provisional application 60/438,778, filed Jan. 9, 2003, which is incorporated by reference in its entirety herein.

### BACKGROUND OF THE INVENTION

1. Field of the Invention

The present invention is in the fields of molecular biology, biochemistry and pharmaceuticals. In general, the invention provides compositions for the cellular delivery of nucleic acids, polypeptides and/or flourophores, molecular complexes comprising fluorescent molecules or moieties, nucleic acids and polypeptides, and methods of making and using such compositions. Light-activated dispersal of the complexes leads to the intracellular release of one or more nucleic acids and/or peptides from the compositions or complexes. The biological activities of nucleic acids, polypeptides and flourophores may be repressed within the complexes, and these activities are restored upon release from the complexes.

2. Related Art

The following description of the background of the invention is provided to aid in understanding the invention, but is not admitted to describe or constitute prior art to the invention. All patents and publications mentioned in the specification are hereby incorporated by reference to the same extent as if each individual patent and publication was specifically and individually indicated to be incorporated by reference.

Translocating proteins are defined by their ability to cross biological membranes. The amino acid sequences that mediate translocation have been referred to as protein transduction domains (PTD). For reviews of translocating proteins and PTD sequences, see Schwartz, J. J., and Zhang, S., Curr Opin Mol Ther. 2:162-167 (2000); and Schwarze, S. R., et al., Trends Cell Biol. 10:290-295 (2000).

When a native translocating protein, or a synthetic protein comprising a PTD, is applied to the medium of cultured mammalian cells, the protein is taken up and may accumulate in the cytoplasm or nucleus of the cell. This translocation may occur in vivo and, in some instances, may allow a protein comprising a PTD to cross the blood brain barrier (BBB).

Translocating proteins and peptides that have been described include but are not limited to the VP22 protein from Herpes Simplex Virus type 1 (Elliott, G., and O'Hare, P., Cell 88:223-233 (1997)), and peptides derived from the HIV Tat protein, the Drosophila homeodomain protein Antennapedia (Derrossi et al., 1994, 1996) or the Kaposi basic FGF receptor (K-FGF) (Rojas et al., 1998; Dokka, S., Pharm Res 14:1759-64 (1997)). In addition, synthetic peptides have been prepared using structural information obtained from naturally-occurring PTDs.

Normand, N., et al., J. Biol. Chem. 276:15042-15050 (2001), assert that when a VP22-derived peptide (corresponding to amino acids 159-301 of the native VP22 protein) and a fluorescently labeled oligonucleotide are mixed, spherical particles are formed that can be taken up by cells and are stable in the cytoplasm for at least 48 hours. Following illumination with light of the appropriate wavelength, the oligonucleotide is released from the complexes and disperses throughout the cell. Using this system, an antisense oligonucleotide directed against the human raf kinase was activated in cells in a light-dependent manner.

Other documents that may comprise information relevant to the invention described herein include without limitation the following:

U.S. Pat. No. 6,342,229 (“Herpes virus particles comprising fusion protein and their preparation and use”), U.S. Pat. No. 6,251,398 (“Materials and methods for intracellular transport and their uses”), U.S. Pat. No. 6,184,038 (“Transport proteins and their uses”), U.S. Pat. No. 6,017,735 (“Materials and methods for intracellular transport and their uses”) and U.S. Pat. No. 6,521,455; published U.S. patent application Nos. US 2002/0064534 A1 (“Herpes virus preparations and their uses”), US 2002/0039765 A1 (“Transport proteins and their uses”), US 2001/0048928 A1 (“Herpes virus particles comprising fusion protein and their preparation and use”), U.S. 2003/0219859A1, US 2002/0142960A, and US 2002/016378; and published PCT Patent Applications WO 02/20060 (“VP22 protein/nucleic acid aggregates, uses thereof”) and related published U.S. application 2002/0142960A1, WO 00/53722 (“Delivery of substances to cells”), WO 98/32866 (“Fusion proteins for intracellular and intercellular transport and their uses”), and WO 97/05265 (“Transport proteins and their uses”), all to O'Hare, et al.

U.S. Pat. No. 6,306,993 (“Method and composition for enhancing transport across biological membranes”) and related U.S. Pat. No. 6,495,663; published U.S. patent application US 2002/0009491; (“Compositions and methods for enhancing drug delivery across biological membranes and tissues”); and published PCT patent applications WO 02/067917 (“Compositions and methods for enhancing drug delivery across and into ocular tissues”); WO 01/62297 (“Compositions and methods for enhancing drug delivery across biological membranes and tissues”); WO 01/13957 (“Enhancing drug delivery across and into epithelial tissues using oligo arginine moieties”), related WO02/069930, U.S. Pat. No. 6,593,292; and U.S. published applications 2003/0083256A1, 2003/0022831A1, and 2002/0127198A1; and WO 98/52614 (“Compositions and method for enhancing transport across biological membranes and tissues”), all to Rothbard, et al.

Published PCT Patent Application WO 00/58488 (“Delivery of functional protein sequences by translocating polypeptides”) to Dalby and Bennett.

Published PCT Patent Application WO 02/065986 and published U.S. application 2003/0032593A1 (“Transporters comprising spaced arginine moieties”) to Wender, et al.

Published PCT Patent Application WO 02/20737 (“Genome Engineering by Cell-Permeable DNA Site-Specific Recombinases”) to Ruley and Jo.

Published U.S application 2002/0120100; and published PCT patent application WO 02/31109 (both entitled “Intracellular delivery of biological effectors”), both to Bonny, et al.

U.S. Pat. Nos. 6,248,558 and 6,432,680; published U.S application 2002/0143142; and published PCT patent application WO 99/49879 (all entitled “Sequence and method for genetic engineering of proteins with cell membrane translocating activity”), all to Lin, et al.

Published U.S application 2002/0132788 (“Inhibition of gene expression by delivery of small interfering RNA to post-embryonic animal cells in vivo”), to Lewis, et al.

Published U.S application 2002/0162126 (“Methods and compositions for RNA interference”) to Beach, et al.

Published U.S application 2002/0086356 (“RNA sequence-specific mediators of RNA interference”) to Tuschl, et al.

Published U.S application 2002/0160393 (“Double-stranded RNA-mediated gene suppression”) to Symonds, et al.

Published U.S application 2002/0137210 (“Method for modifying genetic characteristics of an organism”) to Churikov, et al.

Published U.S application 2002/132346 (“Use of RNA interference for the creation of lineage specific ES and other undifferentiated cells and production of differentiated cells in vitro by co-culture”) to Cibelli et al.

### BRIEF SUMMARY OF THE INVENTION

The present invention provides compositions and non-covalent complexes comprising one or more nucleic acid molecules (e.g., one or more oligonucleotides) and one or more polypeptides. The invention also provides compositions comprising such complexes. One or more fluorescent molecules or moieties, which may be the same or different, and may be covalently attached to one or more polypeptides and/or nucleic acid molecules in the complexes of the invention. Alternatively, or in addition, complexes of the invention may comprise one or more “free” fluorescent molecule (i.e., one or more fluorescent molecules that are not covalently attached to either the polypeptide or the oligonucleotide but may still be associated with the complex). One or more of the compounds of the compositions or complexes can be a biologically active molecule.

Complexes according to the invention or portions thereof, can comprise a cellular delivery molecule that can facilitate the translocation of the complex or portion thereof into cells. In some embodiments, polypeptides for use in the present invention may comprise one or more cellular delivery molecules.

In some embodiments, complexes of the invention include complexes that may dissociate when contacted with an appropriate stimulus. Suitable stimuli include, but are not limited to, electromagnetic radiation (e.g., light), particularly electromagnetic radiation having a wavelength in the range of wavelengths from about 200 nm to about 800 nm. Dissociation of a complex in a cell can make a biologgically active molecule that was within the complex available to function in the cell.

In some embodiments, a cell, tissue, organ or organism may be contacted with a complex of the invention. Preferably, the complex is taken up by the cell or by one or more cells of the tissue, organ or organism. The complex may then be contacted with a suitable stimulus to dissociate the complex. For example, one or more cells, tissues, organs or organisms containing one or more complexes of the invention may be contacted with an extracellular stimulus (e.g., light), resulting in the dissociation of the nucleic acid, polypeptide, and/or fluorescent molecule, or any combination thereof, from the complexes.

In some embodiments, dissociation of one or more of the components of the complex may result in a change in the activity level of the component and/or the complex. In some embodiments, a component that dissociates from a complex may interact with one or more intracellular molecules thereby modulating the activity of the intracellular molecule, and thereby exhibiting biological activity.

The sensitivity of these complexes to an appropriate stimulus allows for the controlled dissociation of one or more of the components of the complex. The dissociation may be controlled, for example, to release one or more of the components at a desired time and/or at a desired location (e.g. intracellularly). Release of a nucleic acid component may stimulate one or more activities associated with the nucleic acid, such as an antisense effect or a ribozyme activity. Release of a polypeptide component may stimulate one or more activities associated with the protein, such as site-specific recombination. Any one or more of the components of a complex may have one or more activities. For example, a polypeptide, a nucleic acid and/or a fluorescent molecule may be an active agent. Alternatively or additionally, the complex can comprise an active agent other than a polypeptide, a nucleic acid and/or a fluorescent molecule, and the agent can be released and activated by an appropriate stimulus (e.g., light).

In one embodiment, the cellular delivery molecule of the complex is a cellular delivery polypeptide. In another embodiment, the active agent is a bioactive polypeptide. If both the active agent and the cellular delivery molecule are polypeptides, a fusion protein may comprise both the cellular delivery polypeptide and the bioactive polypeptide. Complexes comprising this fusion protein can be formed, and the activity of the bioactive polypeptide may be stimulated in a light dependent manner.

In another exemplary and non-limiting embodiment of the invention, compositions comprising complexes between cellular delivery polypeptides and oligonucleotides are formed and can be applied to cultured mammalian cells. Either the cellular delivery polypeptide or the oligonucleotide, or both, is labeled with a fluorescent molecule such as FITC. These complexes allow delivery followed by light activated dispersal of the oligonucleotide and peptide components within the cell. The complex may also comprise a combination of labeled and nonlabeled nucleic acid and or peptide. The light sensitivity of these complexes allows controlled release of an activity associated with the oligonucleotide, which, by way of non-limiting example, can be a gene-containing oligonucleotide, an antisense oligonucleotide, an aptamer, a short interfering RNA (siRNA), a short hairpin RNA (shRNA), a small temporally regulated RNA (stRNA), and the like. In some embodiments, oligonucleotides are preferred.

The invention encompasses a method of delivering a polypeptide to a cell, comprising:

(a) contacting said cell with, in any order or combination, said polypeptide, a nucleic acid, a fluorescent molecule, and a cellular delivery molecule; and

(b) treating said cell with a treatment that results in the dissociation of said polypeptide from one or more of said nucleic acid, said fluorescent molecule, and said cellular delivery molecule.

Preferably step a is conducted such that a complex is formed copmising the cellular delivery molecule and a fluorescent molecular or moiety.

In related embodiments, the method of treatment further comprises irradiation of said cell or a tissue or organism containing the cell. The irradiation typically involves electromagnetic radiation at a wavelength of from about 200 nm to about 800 nm. The treatment may additionally or alternatively comprise a treatment, such as contacting with chloroquine, that disrupts endosomes. In this and other embodiments described herein, the polypeptide, nucleic acid, fluorescent molecule, and/or the cellular delivery molecule can be admixed to form complexes before said contacting.

In some embodiments, the cellular delivery molecule is a cellular delivery polypeptide. The cellular delivery polypeptide can have an amino acid sequence that is derived from the amino acid sequence of a protein encoded by a retrovirus, a prokaryote, a bacteriophage, an archea, an archeal virus, or a eukaryotic cell. In other embodiments, the cellular delivery polypeptide is derived from a homeobox gene product, including by way of non-limiting example the Drosophila Antennapedia protein (Antp). The cellular delivery polypeptide can be a synthetic peptide. The synthetic peptide may comprise one or more unnatural amino acids, such as Ornithine (Orn). Exemplary synthetic peptides that can be used to practice the invention include without limitation those described herein. In specific embodiments, the cellular delivery polypeptide can be a polypeptide having 9 or more amino acids in which 5 or more the amino acids are argenines.

In other specific embodiments, the cell delivery polypeptide is covalently labeled with a fluorophores (fluorescent moiety), for example with fluorescein or a derivative of fluorescein. The peptide may be labeled at its N-terminus or at its C-terminus. In more specific embodiments, the cell delivery polypeptide is covalently labeled to a fluorophore through a linker group. The length of the linker group and the functional groups of the linker group may be varied. The linker may be a carboxyamide linker or a thiourea linker. The length of the linker group can be adjusted as is known in the art by introduction of a spacer group (e.g., a —(CH₂)ₓ-group (where x is an integer, e.g., an integer from 1-about 10) or an ether or polyether spacer, preferably being 1-10 atoms in length.

The cellular delivery polypeptide may be comprised within a fusion protein that further comprises other elements, such as an accessory polypeptide. An accessory polypeptide can be, by way of non-limiting example, an accessory element (e.g., an affinity tag, a purification element, an epitope, a protease cleavage site, or an intracellular targeting element); a bioactive polypeptide (e.g., an enzyme, a detectable polypeptide, a hormone, a growth factor, an antibody or antibody derivative, etc.). The enzyme may be one that has a nucleic acid as one of its reactants or one of its products (e.g., a recombinase, such as a site-specific recombinase. In such embodiments, it may be desirable to include at least one site recognized by the site-specific recombinase in the nucleic acid.

The invention provides a method of delivering a polypeptide to a cell, wherein said cellular delivery polypeptide:

(a) comprises m% basic amino acids, wherein m % is from about 50% to 100%;

(b) comprises a sequence of n contiguous basic amino acids, wherein n is any whole integer between 2 and about 75; and, additionally or alternatively,

(c) has an amino acid sequence that is not present in the amino acid sequence of a protein encoded by herpes simplex virus (HSV).

Generally, m % can be from about 50% to 100%, from about 55% to about 95%, from about 60% to about 90%, from about 65% to about 85%, from about 70% to about 80%, about 75%, from about 65% to 100%, from about 70% to 100%, from about 75% to 100%, from about 80% to 100%, from about 85% to 100%, from about 90% to 100%, from about 95% to 100%, about 99%, 100%, from about 55% to about 90%, from about 55% to about 85%, from about 55% to about 80%, from about 55% to about 75%, from about 55% to about 70%, from about 55% to about 65%, or from about 55% to about 60%.

In some embodiments, the cellular delivery polypeptide has a pI from about 10.5 to 14, and/or an oligopeptide having the sequence of the n contiguous basic amino acids has a pI from about 10.5 to 14. In other embodiments, the cellular delivery polypeptide has a pI or greater than about 12.0 and/or the cellular delivery polypeptide can also be an oligopeptide comprising a sequence of n contiguous amino acids having a pI of 12 or more. In general, the pI for the cellular delivery polypeptide and/or an oligopeptide having the sequence of the n contiguous basic amino acids can range from a lower value of about 9.5, about 10, about 10.5, about 11, about 11.5, about 12, about 12.5, about 13 or about 13.5 to an upper value of about 13.5, about 14, about 14.5, about 15, about 15.5, about 16, about 16.5, about 17, or about 17.5, and any intermediate ranges contained within the above ranges (i.e., the range of “from about 10.5 to about 14” encompasses the intermediate ranges of, e.g., “from about 10.5 to about 10.6,” “from about 11.2 to about 13.2,” “from about 13.6 to about 13.9,” etc.).

The cellular delivery molecule, or accessory polypeptide or element, can be a nucleic acid binding protein. By way of non-limiting example, such proteins include histones, histonelike proteins, poly-Lysine, poly-Arginine and combinations and derivatives thereof.

In another embodiment, the invention provides a method of delivering a polypeptide to a cell, comprising

(a) contacting said cell with, in any order or combination, said polypeptide, a nucleic acid, a fluorescent molecule, a cellular delivery molecule, and a transfection agent; and

(b) treating said cell with a treatment that results in the dissociation of said polypeptide from one or more of said nucleic acid, said fluorescent molecule, and said cellular delivery molecule.

In another embodiment, the invention provides a kit comprising at least one fluorescent molecule and at least one cellular delivery molecule. In the kit, one or both of the cellular delivery molecule and the fluorescent molecule may be polypeptides and may be comprised within a single fusion protein.

Kits according to the invention may further comprise one or more transfection agents, one or more cells, one or more nucleic acids, one or more set of instructions, and one or more photoilluminators and, optionally, a power supply therefore, or means for connecting one or more of the kit components to a power supply. Batteries and solar panels are representative power supplies.

In a specific embodiment, a kit contains at leastone cell delivery molecule and components for fluorescently labeling the cell delivery molecule.

Other additional kit components include without limitation: additional nucleic acids, such as oligonucleotides, iRNA molecules, plasmids, etc.; one or more transfection agents, such as those described in Table 4; one or more recombinases, including without limitation site-specific recombinases; one or more recombination proteins; and/or one or more cells. In some embodiments, the cells are competent for transfection or transformation, and may express or overexpress dicer.

In other embodiments, the invention provides a complex comprising a cellular delivery polypeptide and an agent that is desirably taken up by cells, wherein the cellular delivery polypeptide comprises a fluorescent moiety. The activity of the agent that is desirably taken up by cells can be an activity that is repressed within the complex but is activated once said agent dissociates therefrom.

The nucleic acid of the complexes and other embodiments of the invention can comprise from 5 bases to about 200 kilobases. Any type of nucleic acid may be used, including by way of non-limiting example mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof. Reviews of tmRNA include Muto A, Ushida C, Himeno H. A bacterial RNA that functions as both a tRNA and an mRNA. Trends Biochem Sci. 1998 January;23(1):25-9; and Withey J H, Friedman D I. The biological roles of trans-translation. Curr Opin Microbiol. 2002 April;5(2):154-9). The nucleic acid may comprise one or more chemical modifications.

A complex according to the invention may further comprise one or more transfection agents, one or more recombinases and, additionally or alternatively, one or more recombination proteins.

In another embodiment, the invention provides a method of delivering a polypeptide to a cell, the method comprising:

(a) contacting said cell with, in any order or combination, said nucleic acid, a fluorescent molecule, and a cellular delivery molecule; and

(b) treating said cell with a treatment that results in the dissociation of said nucleic acid from one or both of said fluorescent molecule and said cellular delivery molecule.

In some embodiments, the treatment in (b) comprises irradiation. The nucleic acid is preferably dispersed in the cytoplasm of said cell, and/or becomes biologically active, following the treatment.

In the methods and compositions of the invention, the fluorescent molecule need not be attached to the nucleic acid; it can also be attached to the cellular delivery molecule, another agent (such as a transfection agent) used in the methods, or can be non-covalently associated with one or more other components of, or additions to, the complexes.

The cellular delivery polypeptide may be a synthetic peptide. The synthetic peptide may comprise one or more unnatural amino acids, such as Ornithine (Orn). Exemplary synthetic peptides that can be used to practice the invention include without limitation those described herein.

In some embodiments, the cellular delivery polypeptide:

(a) comprises m % basic amino acids, wherein m % is from about 50% to 100%;

(b) comprises a sequence of n contiguous basic amino acids, wherein n is any whole integer between 2 and about 75; and, additionally or alternatively,

(c) has an amino acid sequence that is not present in the amino acid sequence of a protein encoded by herpes simplex virus (HSV).

A nucleic acid used in the invention includes, in some embodiments, a sequence that encodes a protein or a portion thereof. In some embodiments, a cellular nucleic acid encoding the protein, or a portion thereof, is desirably replaced by said sequence in one form of gene therapy. Additionally or alternatively, the protein is expressed in the cell. The protein may be exogenous or endogenous. In the latter case, the cells to be transfected may comprise a non-functional form of said protein.

In another embodiment, the invention provides a method of delivering a polypeptide to a cell, comprising:

(a) contacting said cell with, in any order or combination, said nucleic acid, a fluorescent molecule, a cellular delivery molecule, and a transfection agent; and

(b) treating said cell with a treatment that results in the dissociation of said nucleic acid from one or more of said fluorescent molecule and said cellular delivery molecule.

In another embodiment, the invention provides a molecular complex comprising one or more nucleic acids, one or more fluorophores, and one or more cellular delivery polypeptides, wherein each cellular delivery polypeptide:

(a) is m % basic amino acids, wherein m is from about 10% to 100%;

(b) comprises a sequence of n contiguous basic amino acids, wherein n is any whole integer between 2 and 50; and

(c) is not derived from a herpes simplex virus (HSV) protein.

In related embodiments, a composition comprises the molecular complex, and optionally further comprises a transfection agent, a transfection enhancing agent, and/or an endosome disrupting agent. Also provided are cells comprising the molecular complex, and compositions comprising such cells. In certain embodiments, including kit embodiments, the cells preferably remain viable even if the composition is frozen and/or dried. Accordingly, the composition may further comprise an agent such as glycerol. Compositions of the invention may be held within a container. Typically, the container is sealed, and composed of a material that is opaque. The container can be open in formats such as, by way of non-limiting example, microtiter plates. Generally, the microtiter plate is composed of an opaque material and is covered by an opaque film, such as aluminum foil.

A composition of the invention may be a pharmaceutical composition. In certain embodiments, one or more of the nucleic acid, the polypeptide and/or the fluorophore has a biological activity, including but not limited to therapeutic activity. By way of non-limiting example, biologically active nucleic acids are selected from the group consisting of mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof.

Additionally or alternatively, the polypeptide of the complex is biologically active. A biologically active polypeptide may be a therapeutic protein. By way of non-limiting example, bioactive proteins include antibodies or antibody fragments, hormones, enzymes, transcription factors, growth factors, and the like.

The invention further provides a method of providing gene therapy to an individual in need thereof, of treating an individual suffering from a disease or disorder, the method comprising contacting the individual, or cells therefrom, with one or more complexes, compositions and/or pharmaceutical compositions of the invention.

The invention further provides a method of testing a cellular response to a test compound, the method comprising:

(a) contacting a first cell with, in any order or combination, a first nucleic acid, a fluorescent molecule, and a cellular delivery molecule;

(b) contacting a second cell with, in any order or combination, a second nucleic acid, said fluorescent molecule, and said cellular delivery molecule;

(c) treating said cell with a treatment that results in the dissociation of said polypeptide from one or more of said nucleic acid, said fluorescent molecule, and said cellular delivery molecule;

(d) contacting said cells with said test compound, before (a); during (a), (b) or (c); between (a) and (b); between (b) and (c); and, additionally or alternatively, after (c);

(e) detecting a signal from said cells, wherein said signal corresponds to a cellular response; and

(f) comparing the signal from said first cell with the signal from said second cell.

In certain embodiments, one or more of the cells comprise one or more reporter genes that generate a detectable signal or interfere with the production of a detectable signal.

The invention further provides a method of identifying a compound having a preselected activity or effect, the method comprising:

(a) contacting a first cell with, in any order or combination, a first nucleic acid, a fluorescent molecule, and a cellular delivery molecule;

(b) contacting a second cell with, in any order or combination, a second nucleic acid, a fluorescent molecule, and a cellular delivery molecule;

(c) treating said cell with a treatment that results in the dissociation of said polypeptide from one or more of said nucleic acid, said fluorescent molecule, and said cellular delivery molecule;

(d) contacting said cells with said test compound, before (a); during (a), (b) or (c); between (a) and (b); between (b) and (c); and, additionally or alternatively, after (c);

(e) detecting a signal from said cells, wherein said signal corresponds to a cellular response; and

(f) comparing the signal from said first cell with the signal from said second cell,

wherein a difference in the signal from first cell from the signal from said second cell corresponds to said preselected activity or effect.

Other features and advantages of the invention will be apparent from the following detailed description and claims.

### BRIEF DESCRIPTION OF THE DRAWINGS

FIG. 1 shows the time course of photoactivated redistribution of a [FITC-labeled oligonucleotide:R9 peptide] complex in CHO cells.

FIG. 2 shows BrdU labeling of cells treated with anti-raf oligo delivered using peptide R9 or VP22. The staining of cell nuclei indicates BrdU incorporation due to active DNA synthesis and cell cycle progression. Symbols: “+” indicates cells were illuminated for 5 min, “−” indicates cells were not illuminated.

FIG. 3 shows light dependent activation of a lacZ reporter gene using a VP22-Cre/FITC oligo complex. Panel A shows the DNA constructs used in the experiments. In these constructs, the lacZ ORF is separated from the CMV promoter by an intervening sequence containing a transcriptional terminator (int). Cre allows recombination between the lox sites and expression of lacZ. Panel B shows the post-illumination pattern of expression of lacZ (dark patched in upper part of the panel). A distinct boundary between illuminated and unilluminated cells can be seen in cells transiently transfected with the lacZ reporter gene.

### DETAILED DESCRIPTION OF THE INVENTION

I. Definitions and Abbreviations

In the description that follows, a number of terms used in molecular biology and medical/pharmaceutical sciences are utilized extensively. In order to provide a clear and consistent understanding of the specification and claims, including the scope to be given such terms, the following definitions are provided. Under these definitions, the following terms have the following meaning unless otherwise specified herein:

Amplification: As used herein, amplification is any in vitro method for increasing a number of copies of a nucleotide sequence with the use of one or more polypeptides having polymerase activity (e.g., one or more nucleic acid polymerases or one or more reverse transcriptases). Nucleic acid amplification results in the incorporation of nucleotides into a DNA and/or RNA molecule or primer thereby forming a new nucleic acid molecule complementary to a template. The formed nucleic acid molecule and its template can be used as templates to synthesize additional nucleic acid molecules. As used herein, one amplification reaction may consist of many rounds of nucleic acid replication. DNA amplification reactions include, for example, polymerase chain reaction (PCR). One PCR reaction may consist of 5 to 100 cycles of denaturation and synthesis of a DNA molecule.

Association: the covalent or non-covalent joining of two or more molecules, which may occur permanently, temporary, or transiently. A molecular complex is formed by the stable or semi-stable association of two or more compounds.

Base Pair (bp): a partnership of adenine (A) with thymine (T), or of cytosine (C) with guanine (G) in a double stranded DNA molecule. In RNA, uracil (U) is substituted for thymine. Base pairs are said to be “complementary” when their component bases pair up normally when a DNA or RNA molecule adopts a double stranded configuration.

Blocking Agent: a nucleotide (or derivatives thereof), modified oligonucleotides and/or one or more other modifications which are incorporated into nucleic acid inhibitors of the invention to prevent or inhibit degradation or digestion of such nucleic acid molecules by nuclease activity. One or multiple blocking agents may be incorporated in the nucleic acid inhibitors of the invention internally, at or near the 3′ termini and/or at or near the 5′ termini of the nucleic acid inhibitors. Preferably, such blocking agents are located, for linear inhibitor nucleic acid molecules, at or near the 3′ termini and/or at or near the 5′ termini and/or at the preferred cleavage position of the 5′ to 3′ exonuclease of such molecules (Lyamichev, V., Brow, M. A. D., and Dahlberg, J. E., Science 260:778-783 (1993)). Preferably, such blocking agents prevent or inhibit degradation or digestion of the inhibitor nucleic acid molecules by exonuclease activity associated with the polymerase or reverse transcriptase used or that may be present in the synthesis reaction. For example, blocking agents for the invention prevent degradation or digestion of inhibitor nucleic acid molecules by 3′ exonuclease activity and/or 5′ exonuclease activity associated with a polymerase (e.g., a DNA polymerase). Blocking agents include, but are not limited to, dideoxynucleotides and their derivatives such as ddATP, ddCTP, ddGTP, ddITP, and ddTTP; AZT; phosphorothioate backbones; phosphamide backbones (e.g., PNAs), 3′-dNTPs (e.g., Condycepin) or any nucleotide containing a blocking group, preferably at its 3′-position. Such blocking agents preferably act to inhibit or prevent exonuclease activity (e.g., 3′-exonuclease activity) from altering or digesting the inhibitory nucleic acids of the invention. In some embodiments, the 5′-terminal of the oligonucleotides of the present invention may be modified in order to make them resistant to 5′-to-3′ exonuclease activity. One such modification may be to add an addition nucleotide to the 5′-end of the oligonucleotide in a 5′-5′-linkage (see, Koza. M. et al., Journal of Organic Chemistry 56:3757).

Cellular Delivery (also referred to herein interchangeably and equivalently as “delivery”): a process by which a desired compound is transferred to a target cell such that the desired compound is ultimately located inside the target cell, or in or on the target cell membrane. In certain uses delivery to a specific target cell type is preferable.

Cellular Delivery Molecule: a molecule that mediates the Cellular Delivery of itself, a molecular complex comprising the Cellular Delivery Molecule, and/or a molecule comprising the Cellular Delivery Molecule. Preferably, Cellular Delivery Molecules possess one or more of the following properties: resistance to degradation, both in vitro and in vivo; receptor-independent delivery to cells; and substantially energy-free penetration of cell membranes.

Cellular Delivery Polypeptide: a polypeptide that functions as a Cellular Delivery Molecule, either by itself, as a part of a molecular complex, and/or as part of a fusion protein. By way of non-limiting example, Cellular Delivery Polypeptides include translocating proteins having amino acid sequences referred to as protein transduction domains (PTD).

Competent Cells: cells having the ability to take up and establish an exogenous nucleic acid, such as a DNA molecule.

Complementary Nucleotide Sequence: a sequence of nucleotides in a single-stranded molecule of DNA or RNA that is sufficiently complementary to another single strand to specifically (non-randomly) hybridize to it with consequent hydrogen bonding.

Construct: a vector sequence, or a portion thereof, that has been linked with one or more non-vector sequences.

Dissociation: the separation of two or more molecules in association with each other, and/or the release of one or more molecules from a molecular complex.

DNA molecule: any DNA molecule, of any size, from any source, including DNA from viral, prokaryotic, and eukaryotic organisms. The DNA molecule may be in any form, including, but not limited to, linear or circular, and single or double stranded. Non-limiting examples of DNA molecules include plasmids, vectors, and expression vectors

Expression: the process by which a gene produces a polypeptide. It includes transcription of the gene into messenger RNA (mRNA) and the translation of such mRNA into polypeptide(s).

Fusion Protein: a polypeptide comprising two distinct proteins, polypeptides, peptides, and/or fragments thereof that are not normally encoded by the same ORF. In order to produce a fusion protein, genetic engineering is used to prepare a nucleic acid having an ORF (a chimeric ORF) comprising nucleotide sequences encoding the two or more distinct proteins, polypeptides, peptides, and/or fragments thereof. The fusion protein is the polymer of amino acids that results from the translation of the chimeric ORF. A fusion protein may further include sequences that function for detection and/or purification of the fusion protein, (e.g., protein tags). The fusion protein may further contain sequences that function in the selective cleavage of the fusion protein.

Gene: a DNA sequence that contains information necessary for expression of a polypeptide or protein. It includes the promoter and the structural gene as well as other sequences involved in expression of the protein. The term “structural gene” as used herein refers to a DNA sequence that is transcribed into messenger RNA that is then translated into a sequence of amino acids characteristic of a specific polypeptide.

Host: any prokaryotic, eukaryotic or archeabacterial microorganism or cell that is the recipient of a replicable expression vector, cloning vector or any nucleic acid molecule including the inhibitory nucleic acid molecules of the invention. The nucleic acid molecule may contain, but is not limited to, a structural gene, a promoter and/or an origin of replication. The term “recombinant host” as used herein refers to any prokaryotic or eukaryotic microorganism which contains the desired cloned genes in an expression vector, cloning vector or any other nucleic acid molecule. The term “recombinant host” is also meant to include those host cells which have been genetically engineered to contain the desired gene on a host chromosome or in the host genome. As used herein, the term “host” may be used interchangeably and equivalently with the term “host cell.” Similarly, as used herein, the term “recombinant host” may be used interchangeably and equivalently with the term “recombinant host cell.”

Incorporating: becoming a part of a DNA and/or RNA molecule or primer.

Inducer: a molecule that triggers gene transcription by binding to a regulator protein such as a repressor.

Induction: the switching on of transcription as a result of interaction of an inducer with a positive or negative regulator.

Insert or Inserts: a desired nucleic acid segment or a population of nucleic acid segments that may be manipulated by the methods of the present invention. Thus, the terms Insert(s) are meant to include a particular nucleic acid (preferably DNA) segment or a population of segments. Such Insert(s) can comprise one or more genes.

Insert Donor: one of the two parental nucleic acid molecules (e.g. RNA or DNA) of the present invention which carries the Insert. The Insert Donor molecule comprises the Insert flanked on both sides with recombination sites. The Insert Donor can be linear or circular. In one embodiment of the invention, the Insert Donor is a circular DNA molecule and further comprises a cloning vector sequence outside of the recombination signals. When a population of Inserts or population of nucleic acid segments are used to make the Insert Donor, a population of Insert Donors result and may be used in accordance with the invention.

Molecular Complex (Complex): an aggregate of two or more molecules that is held together by covalent and/or non-covalent bonds. The formation and maintenance of a complex may be dependent on conditions such as pH, temperature, concentration or nature of one or more compounds in a composition comprising the complex, and the like. A “protein complex” is a molecular complex that comprises two or more distinct polypeptides.

Negative Regulation of Transcription: a mechanism of control of gene expression where a gene is transcribed unless transcription is prevented by the action of a negative regulator, or repressor.

Nucleotide: a base-sugar-phosphate combination. Nucleotides are monomeric units of a nucleic acid sequence (DNA and RNA). Nucleotides may also include mono-, di- and triphosphate forms of such nucleotides. The term nucleotide includes ribonucleoside triphosphates ATP, UTP, ITP, CTG, GTP and deoxyribonucleoside triphosphates such as dATP, dCTP, dITP, dUTP, dGTP, dTTP, or derivatives thereof.

Such derivatives include, for example, [aS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels. Various labeling methods known in the art can be employed in the practice of this invention.

Nucleotide Analog: a purine or pyrimidine nucleotide that differs structurally from an A, T, G, C, or U base, but is sufficiently similar to substitute for the normal nucleotide in a nucleic acid molecule. Inosine (I) is a nucleotide analog that can hydrogen bond with any of the other nucleotides, A, T, G, C, or U. In addition, methylated bases are known that can participate in nucleic acid hybridization. Methods of preparing and using modified oligonucleotides are described in: Verma S, Eckstein F. Modified oligonucleotides: synthesis and strategy for users. Annu Rev Biochem. 1998;67:99-134. By way of non-limiting example, nucleotide analogs include 2,6-diamino purine, 6-methyladenine, 8-azaguanine, 5-bromouracil, 5-hydroxymethyl uracil, 5-methylcytosine (5MC), 5-hydroxymethylcytosine (HMC), 8-chloroadenosine, glycosyl HMC, and gentobiosyl HMC. Fluorescent nucleotide analogs, such as those described by Jameson and Eccleston (Fluorescent nucleotide analogs: synthesis and applications. Methods Enzymol. 1997;278:363-90), and cyclic nucleotide analogs, such as those described by Schwede et al. (Cyclic nucleotide analogs as biochemical tools and prospective drugs. Pharmacol Ther 2000 87(2-3):199-226) may also be used in the invention.

Operably Linked: As used herein, the phrase “operably linked” refers to a linkage in which a first nucleotide sequence is connected to one or more second nucleotide sequences in such a way as to be capable of altering the functioning of the second sequence(s). For example, a protein coding sequence which is “operably linked” to a promoter/operator places expression of the protein coding sequence under the influence or control of these promoter/operator sequences. Two nucleotide sequences (such as a protein encoding sequence and a promoter region sequence linked to the 5′ end of the encoding sequence) are said to be operably linked if induction of promoter function results in the transcription of the protein encoding sequence mRNA and if the nature of the linkage between the two nucleotide sequences results in neither (1) the introduction of a frame-shift mutation nor (2) prevention of the regulatory sequences from directing the expression of the mRNA or protein. Thus, a promoter region is said to be “operably linked” to a nucleotide sequence if the promoter is capable of effecting transcription of that nucleotide sequence. As one of ordinary skill will appreciate, two nucleic acid sequences (such as a promoter/operator sequence and a protein encoding sequence) may be operably linked without necessarily being physically located adjacent to one another; so long as the promoter/operator sequence is capable of directing the expression of the protein encoding sequence, the sequences are said to be operably linked regardless of whether the two sequences are located immediately next to each other on the same nucleic acid molecule or are located distal to one another with one or more intervening sequences located between them.

Operator: As used herein, an operator is an example of a transcriptional regulatory sequence, and specifically is the site on DNA at which a repressor protein binds to prevent transcription from initiating at the adjacent promoter.

Photoilluminator: any energy source capable of providing electromagnetic energy (e.g., light) having an appropriate wavelength (typically a wavelength falling within a range of wavelengths from about 200 nm to about 800 nm and intensity for a period of time sufficient to bring about dissociation of the complexes of the invention, thereby disbursing one or more components of the complex into a cell, tissue, organ or organism that has been contacted with one or more complexes of the invention.

Nucleic Acid: As used herein “nucleic acid” and its grammatical equivalents will include the full range of polymers of single or double stranded nucleotides. A nucleic acid typically refers to a polynucleotide molecule comprised of a linear strand of two or more nucleotides (deoxyribonucleotides and/or ribonucleotides) or variants, derivatives and/or analogs thereof. The exact size will depend on many factors, which in turn depends on the ultimate conditions of use, as is well known in the art. The nucleic acids of the present invention include without limitation primers, probes, oligonucleotides, vectors, constructs, plasmids, genes, transgenes, genomic DNA, cDNA, PCR products, restriction fragments, and the like.

Positive Regulation of Transcription: a mechanism of control of gene expression where a gene is transcribed poorly or not at all unless a positive regulator (an “activator”) stimulates or allows, respectively, initiation of transcription.

Primer: a single stranded or double stranded oligonucleotide that is extended by covalent bonding of nucleotide monomers during amplification or polymerization of a nucleic acid molecule (e.g. a DNA molecule). In a preferred aspect, the primer comprises one or more recombination sites or portions of such recombination sites.

Portions of recombination sties comprise at least 2 bases, at least 5 bases, at least 10 bases or at least 20 bases of the recombination sites of interest. When using portions of recombination sites, the missing portion of the recombination site may be provided by the newly synthesized nucleic acid molecule. Such recombination sites may be located within and/or at one or both termini of the primer. Preferably, additional sequences are added to the primer adjacent to the recombination site(s) to enhance or improve recombination and/or to stabilize the recombination site during recombination. Such stabilization sequences may be any sequences (preferably G/C rich sequences) of any length. Preferably, such sequences range in size from 1 to about 1000 bases, 1 to about 500 bases, and 1 to about 100 bases, 1 to about 60 bases, 1 to about 25, 1 to about 10, 2 to about 10 and preferably about 4 bases. Preferably, such sequences are greater than 1 base in length and preferably greater than 2 bases in length.

Promoter: As used herein, a promoter is an example of a transcriptional regulatory sequence, and specifically is a DNA sequence generally described as the 5′-region of a gene located proximal to the start codon. The transcription of an adjacent DNA segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Recombinant DNA (rDNA) Molecule: a DNA molecule produced by operatively linking a nucleic acid sequence, such as a gene, to a DNA molecule sequence of the present invention. Thus, a recombinant DNA molecule is a hybrid DNA molecule comprising at least two nucleotide sequences not normally found together in nature. Different rDNAs, not having a common biological origin (i.e., are evolutionarily distinct) are said to be “heterologous.” It should be noted that, as used herein, “rDNA” does not refer to a DNA that serves as a template for ribosomal RNA (rRNA).

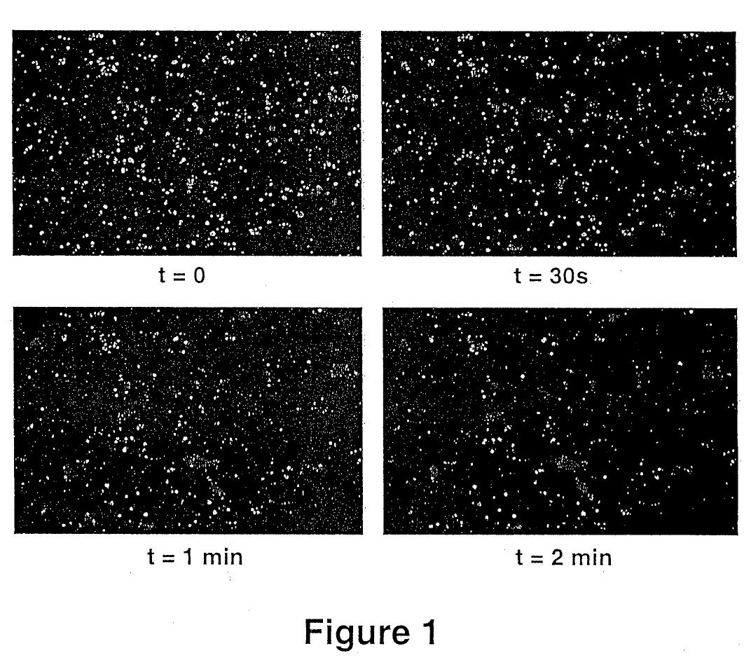

Recognition sequence: As used herein, a recognition sequence is a particular sequence to which a protein, chemical compound, DNA, or RNA molecule (e.g., restriction endonuclease, a modification methylase, or a recombinase) recognizes and binds. In the present invention, a recognition sequence will typically, but need not, refer to a recombination site. For example, the recognition sequence for Cre recombinase is loxP which is a 34 base pair sequence comprised of two 13 base pair inverted repeats (serving as the recombinase binding sites) flanking an 8 base pair core sequence. See FIG. 1 of Sauer, B., Current Opinion in Biotechnology 5:521-527 (1994). Other examples of recognition sequences are the attB, attP, attL, and attR sequences which are recognized by the recombinase enzyme Integrase. The attB site is an approximately 25 base pair sequence containing two 9 base pair core-type Int binding sites and a 7 base pair overlap region. The attP site is an approximately 240 base pair sequence containing core-type Int binding sites and arm-type Int binding sites as well as sites for the auxiliary proteins integration host factor (IHF), FIS and excisionase (Xis). See Landy, Current Opinion in Biotechnology 3:699-707 (1993). Such sites may also be engineered according to the present invention to enhance production of products in the methods of the invention. When such engineered sites lack the P1 or H1 domains to make the recombination reactions irreversible (e.g., attR or attP), such sites may be designated attR′ or attP′ to indicate that the domains of these sites have been modified in some way.

Recombinase: an enzyme which catalyzes the exchange of DNA segments at specific recombination sites.

Recombinational Cloning: a method whereby segments of nucleic acid molecules or populations of such molecules are exchanged, inserted, replaced, substituted or modified, in vitro or in vivo. See U.S. Pat. Nos. 5,888,732; 6,143,557; 6,171,861; 6,270,969; and 6,277,608; the disclosures of all of which are incorporated herein by reference in their entireties.

Recombination proteins: As used herein, recombination proteins include excisive or integrative proteins, enzymes, co-factors or associated proteins (e.g., IHF and/or other histonelike proteins) that are involved in recombination reactions involving one or more recombination sites. Recombination proteins may be wild-type proteins or mutants, derivatives, fragments, or variants thereof.

Recombination site: A used herein, a recombination site is a recognition sequence on a nucleic acid molecule participating in an integration/recombination reaction by recombination proteins. Non-limiting examples of recognition sequences include the attB, attP, attL, and attR sequences described herein, and mutants, fragments, variants and derivatives thereof, which are recognized by the recombination protein λ Int and by the auxiliary proteins integration host factor (IHF), FIS and excisionase (Xis). See Landy, Curr. Opin. Biotech. 3:699-707 (1993).

Reporter gene: a nucleic acid encoding a readily assayable protein. The assays can be qualitative, quantitative, manual, automated, semi-automated, etc. By way of non-limiting example, reporter genes include genes encoding β-galactosidase (lacZ), neomycin resistance, HIS3, luciferase (LUC), chloramphenicol acetyltransferase (CAT), β-glucuronidase (GUS), human growth hormone (hGH), alkaline phosphatase (AP), secreted alkaline phosphatase (SEAP), and fluorescent polypeptides such as GFP. Those skilled in the art will be able to select reporter genes appropriate for the host cell and application of interest. For reviews of vectors and reporter genes see Baneyx F. Recombinant protein expression in Escherichia coli. Curr Opin Biotechnol 10:411-421, 1999; Van Craenenbroeck K, Vanhoenacker P, Haegeman G. Episomal vectors for gene expression in mammalian cells. Eur. J. Biochem. 2000 September;267(18):5665-78; Soll D R, Srikantha T. Reporters for the analysis of gene regulation in fungi pathogenic to man. Curr Opin Microbiol. 1998 August;1(4):400-5; Possee R D. Baculoviruses as expression vectors. Curr Opin Biotechnol. 1997 October;8(5):569-72; and Mount R C, Jordan B E, Hadfield C. Reporter gene systems for assaying gene expression in yeast. Methods Mol Biol. 1996;53:239-48.

Repression: the inhibition of transcription effected by the binding of repressor protein to a specific site on DNA.

Repression Cassette: a nucleic acid segment that contains a repressor of a Selectable marker present in the subcloning vector.

Repressor: a protein which prevents transcription by binding to a specific site on DNA.

Selectable Marker: a DNA segment that allows one to select for or against a molecule or a cell that contains it, of ten under particular conditions. These markers can encode an activity, such as, but not limited to, production of RNA, peptide, or protein, or can provide a binding site for RNA, peptides, proteins, inorganic and organic compounds or compositions and the like. Examples of Selectable markers include but are not limited to: (1) DNA segments that encode products which provide resistance against otherwise toxic compounds (e.g., antibiotics); (2) DNA segments that encode products which are otherwise lacking in the recipient cell (e.g, tRNA genes, auxotrophic markers); (3) DNA segments that encode products which suppress the activity of a gene product; (4) DNA segments that encode products which can be readily identified (e.g., phenotypic markers such as β-galactosidase, green fluorescent protein (GFP), and cell surface proteins); (5) DNA segments that bind products which are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g., restriction endonucleases); (8) DNA segments that can be used to isolate or identify a desired molecule (e.g specific protein binding sites); (9) DNA segments that encode a specific nucleotide sequence which can be otherwise non-functional (e.g. for PCR amplification of subpopulations of molecules); (10) DNA segments, which when absent, directly or indirectly confer resistance or sensitivity to particular compounds; and/or (11) DNA segments that encode products which are toxic in recipient cells.

Selection Scheme: any method which allows selection, enrichment, or identification of a desired Construct or Constructs from a mixture containing one or more DNA inserts Vectors, undesirable alternative Constructs, and reagents, intermediates and/or byproducts from the processes used to generate Constructs. The selection schemes of one embodiment have at least two components that are either linked or unlinked during cloning. One component is a Selectable marker; the other component controls the expression in vitro or in vivo of the Selectable marker, or survival of the cell harboring the Construct carrying the Selectable marker. Generally, this controlling element will be a repressor or inducer of the Selectable marker, but other means for controlling expression of the Selectable marker can be used. Whether a repressor or activator is used will depend on whether the marker is for a positive or negative selection, and the exact arrangement of the various DNA segments, as will be readily apparent to those skilled in the art. A preferred requirement is that the selection scheme results in selection of or enrichment for only one or more desired Constructs. As defined herein, selecting for a DNA molecule includes (a) selecting or enriching for the presence of the desired DNA molecule, and (b) selecting or enriching against the presence of DNA molecules that are not the desired DNA molecule.

Site-Specific Recombinase: a type of recombinase which typically has at least the following four activities (or combinations thereof): (1) recognition of one or two specific nucleic acid sequences; (2) cleavage of said sequence or sequences; (3) topoisomerase activity involved in strand exchange; and (4) ligase activity to reseal the cleaved strands of nucleic acid. See Sauer, B., Current Opinions in Biotechnology 5:521-527 (1994). Conservative site-specific recombination is distinguished from homologous recombination and transposition by a high degree of specificity for both partners. The strand exchange mechanism involves the cleavage and rejoining of specific DNA sequences in the absence of DNA synthesis (Landy, A., Ann. Rev. Biochem. 58:913-949 (1989)).

Substantially pure: as used herein, “substantially pure” means that the desired purified molecule such as a protein or nucleic acid molecule (including the inhibitory nucleic acid molecule of the invention) is essentially free from contaminants which are typically associated with the desired molecule. Contaminating components may include, but are not limited to, compounds or molecules which may interfere with the inhibitory or synthesis reactions of the invention, and/or that degrade or digest the inhibitory nucleic acid molecules of the invention (such as nucleases including exonucleases and endonucleases) or that degrade or digest the synthesized or amplified nucleic acid molecules produced by the methods of the invention

Target Cell: any cell to which a desired compound is delivered. Cells to which the delivery methods of this invention can be applied include cells in vitro, cells ex vivo or cells in vivo. Target cells may be in cell culture, on tissue culture, in any form of immobilized state, or grown on liquid, semi-solid or solid medium.

Target cells may be in the form of a monolayer. Target cells may be collected from an organism and/or cultured by any known method. Target cells include cells without cell walls and cells from which cell walls have been removed by any known treatment (e.g., formation of protoplasts) from which viable cells can be recovered.

Transcriptional regulatory sequence: As used herein, transcriptional regulatory sequence is a functional stretch of nucleotides contained on a nucleic acid molecule, in any configuration or geometry, that acts to regulate the transcription of one or more structural genes into messenger RNA. Examples of transcriptional regulatory sequences include, but are not limited to, promoters, enhancers, repressors, and the like. “Transcription regulatory sequence,” “transcription sites” and “transcription signals” may be used interchangeably.

Transfection: the delivery of expressible nucleic acid to a target cell, such that the target cell is rendered capable of expressing said nucleic acid. It will be understood that the term “nucleic acid” includes both DNA and RNA without regard to molecular weight, and the term “expression” means any manifestation of the functional presence of the nucleic acid within the cell including, without limitation, both transient expression and stable expression.

Transfection Agent: any substance which provides significant enhancement of transfection (2-fold or more) over transfection compositions that do not comprise the transfection agent.

Vector: As used herein, a vector is a nucleic acid molecule that provides a useful biological or biochemical property to a nucleic acid sequence or molecule of interest, for example, an Insert, a coding region, etc. Examples include plasmids, phages, autonomously replicating sequences (ARS), centromeres, and other nucleic acid sequences that are able to replicate or be replicated in vitro or in a host cell, or to convey a desired nucleic acid segment to a desired location within a host cell. A vector may comprise various structural and/or functional sequences, for example, one or more restriction endonuclease recognition sites at which the vector sequences can be manipulated in a determinable fashion without loss of an essential biological function of the vector, and into which a nucleic acid fragment can be inserted, for example to bring about its replication and/or cloning. Vectors can further provide primer sites, e.g., for PCR, transcriptional and/or translational initiation and/or regulation sites, recombinational signals, replicons, selectable markers, and other sequences known to those skilled in the art. A vector comprising a nucleic acid insert is a Construct. Thus, a gene therapy construct is a gene therapy vector into which a therapeutic gene has been cloned. Similarly, a construct that expresses an antisense transcript is an “antisense construct.”

Cloning Vector: A plasmid, cosmid, viral, or phage DNA or other DNA molecule which is able to replicate autonomously in a host cell, into which DNA may be spliced without loss of an essential biological function of the vector, in order to bring about its replication and cloning. The cloning vector may further contain a marker suitable for use in the identification of cells transformed with the cloning vector. Markers may be, for example, antibiotic resistance genes, e.g., tetracycline resistance or ampicillin resistance. Clearly, methods of inserting a desired nucleic acid fragment which do not require the use of homologous recombination, transpositions or restriction enzymes (such as, but not limited to, UDG cloning of PCR fragments (U.S. Pat. No. 5,334,575, entirely incorporated herein by reference), T:A cloning, and the like) can also be applied to clone a fragment into a cloning vector to be used according to the present invention. The cloning vector can further contain one or more selectable markers suitable for use in the identification of cells transformed with the cloning vector.

Subcloning Vector: a cloning vector comprising a circular or linear nucleic acid molecule which includes preferably an appropriate replicon. A subcloning vector can also contain functional and/or regulatory elements that are desired to be incorporated into the final product to act upon or with the cloned DNA Insert.

Additionally or alternatively, the subcloning vector can also contain a Selectable marker (preferably DNA).

Expression Vector: A vector similar to a cloning vector but which is capable of enhancing the expression of a gene which has been cloned into it, after transformation into a host. The cloned gene is usually placed under the control of (i.e., operably linked to) certain transcriptional regulatory sequences such as promoter sequences. An expression vector comprising an operably linked nucleic acid insert is an “expression construct.”

Vector Gene: a gene or portion thereof present on a vector, usually included to provide a necessary function to the maintenance of the vector (e.g., genes required for DNA replication) or otherwise included on the vector in order to identify, distinguish or select cells comprising the vector or desired constructs prepared from the vector. A non-limiting example of a Vector Gene is a Selectable Marker.

Biologically Active: As used herein, the term “biologically active” (synonymous with “bioactive”) indicates that a composition or compound itself has a biological effect, or that it modifies, causes, promotes, enhances, blocks, or reduces a biological effect, or which limits the production or activity of, reacts with and/or binds to a second molecule that has a biological effect. The second molecule can, but need not, be endogenous. A “biological effect” may be but is not limited to one that stimulates or causes an immunoreactive response; one that impacts a biological process in a cell, tissue or organism (e.g., in an animal); one that impacts a biological process in a pathogen or parasite; one that generates or causes to be generated a detectable signal; and the like. Biologically active compositions, complexes or compounds may be used in investigative, therapeutic, prophylactic and diagnostic methods and compositions. Biologically active compositions, complexes or compounds act to cause or stimulate a desired effect upon a cell, tissue, organ or organism (e.g., an animal). Non-limiting examples of desired effects include modulating, inhibiting or enhancing gene expression in a cell, tissue, organ, or organism; preventing, treating or curing a disease or condition in an animal suffering therefrom; limiting the growth of or killing a pathogen in an animal infected thereby; augmenting the phenotype or genotype of an animal; stimulating a prophylactic immunoreactive response in an animal; or diagnosing a disease or disorder in an animal.

In the context of investigative applications of the invention, including but not limited to forensic and scientific research applications, the term “biologically active” indicates that the composition, complex or compound has an activity that results, directly or indirectly, in a change in some form of measurable output in materials, biological samples, cells or organisms that have been contacted therewith. Investigative applications may be used to determine the quantity or concentration of a selected target compound in a test sample, to determine the effect of a bioactive compound upon cells or animals, or to screen for compounds having an activity that alters, blocks or augments a selected biological activity.

In the context of therapeutic applications of the invention, the term “biologically active” indicates that the composition, complex or compound has an activity that impacts an animal suffering from a disease or disorder in a positive sense and/or impacts a pathogen or parasite in a negative sense. Thus, a biologically active composition, complex or compound may cause or promote a biological or biochemical activity within an animal that is detrimental to the growth and/or maintenance of a pathogen or parasites; or of cells, tissues or organs of an animal that have abnormal growth or biochemical characteristics, such as cancer cells.

In the context of prophylactic applications of the invention, the term “biologically active” indicates that the composition or compound induces or stimulates an immunoreactive response. In some preferred embodiments, the immunoreactive response is designed to be prophylactic, i.e., to prevent infection by a pathogen. In other preferred embodiments, the immunoreactive response is designed to cause the immune system of an animal to react to the detriment of cells of an animal, such as cancer cells, that have abnormal growth or biochemical characteristics. In this application of the invention, compositions, complexes or compounds comprising antigens are formulated as a vaccine.

In the context of diagnostic applications on the invention, the term “biologically active” indicates that the composition, complex or compound can be used for in vivo or ex vivo diagnostic methods and in diagnostic compositions and kits. For diagnostic purposes, a preferred biologically active composition or compound is one that can be detected, typically (but not necessarily) by virtue of comprising a detectable polypeptide. Antibodies to an epitope found on composition or compound may also be used for its detection.

It will be understood by those skilled in the art that a given composition, complex or compound may be biologically active in therapeutic, diagnostic and/or prophylactic applications. A composition, complex or compound that is described as being “biologically active in a cell” is one that has biological activity in vitro (i.e., in a cell or tissue culture) or in vivo (i.e., in the cells of an animal). A “biologically active component” of a composition or compound is a portion thereof that is biologically active once is liberated from the composition or compound. It should be noted that such a component may also be biologically active as a moiety or other portion of the composition or compound.

In the disclosure and the claims, “and/or” means additionally or alternatively. Moreover, any use of a term in the singular also encompasses plural forms.

Other terms used in the fields of recombinant DNA technology, molecular and cell biology, and the medical/pharmaceutical arts, as used herein, are intended to encompass the broadest scope term understood in the art for a given and will be generally understood by one of ordinary skill in the applicable arts.

TABLE 1
Abbreviations and Suppliers

| Abbreviation | Full Term | Suppliers & Sources |
|---|---|---|
| BrdU | Bromo-deoxyuridine | |
| Bp | Base pair(s) | |
| CHO cells | Chinese hamster ovary cells | ATCC CCL-61 (CHO-K1) |
| DMEM | Dulbecco's Modified Eagle | Invitrogen Corporation, |
| | Medium | Carlsbad, CA |
| D-PBS | Dulbecco's Phosphate- | Invitrogen |
| | Buffered Saline | |
| FITC | Fluorescein isothiocyanate | Molecular Probes, Inc., |
| | | Eugene, OR |
| GFP | green fluorescent protein | |
| kbp | Kilobase(s) or kilobase pairs | |
| HAM | Ham's F-12 media | Invitrogen |
| ORF | open reading frame | |
| ORN | ornithine | |
| pI | “Isoelectric focusing point” | |
| PBS | Phosphate Buffered Saline | Invitrogen |
| PTD | protein transduction domain | |
| RNAi | RNA interference | |
| shRNA | short hairpin RNA | |
| siRNA | short interfering RNA | |
| UDG | Uracil DNA glycosylase | |

TABLE 2
Concordance of One- and Three-Letter Codes for Amino Acids

| | Three-letter | One-letter |
|---|---|---|
| Full name | Code | Code |
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic Acid | Glu | E |
| Glycince | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Trypophan | Trp | W |
| Tyrosine | Tyr | Y |
| Valine | Val | V |

II. Overview

In general terms, the present invention provides compositions, complexes and methods for delivering one or more nucleic acids (e.g., one or more nucleic acid molecules, oligonucleotides, polynucleotides, vectors, genes and the like) and/or one or more peptides (e.g., one or more peptides, oligopeptides, polypeptides, proteins or protein complexes) to cells, tissues, organs and whole organisms. The compositions and complexes of the invention typically comprise one or more nucleic acids and one or more proteins or polypeptides (which can be cellular delivery (suitably, translocating) peptides, polypeptides or proteins, such as those described and used in WIPO/PCT publication no. WO 00/58488, the disclosure of which is incorporated herein by reference in its entirety). In certain embodiments, the compositions and complexes of the invention optionally comprise one or more light-activated compounds such as one or more fluorophores, in a complex suitable for delivery of the one or more nucleic acids and/or one or more peptides to the cells, tissues, organs or organisms.

In certain such aspects of the invention, the complexes comprising one or more nucleic acids and/or one or more peptides are delivered to and taken up by the cells, tissues, organs or organisms, and cells, tissues, organs or organisms are then treated with light at a suitable wavelength and intensity to cause photoactivation of the one or more light-activated compounds. This photoactivation results in the release of the one or more nucleic acids and/or one or more peptides from the complexes of the invention such that they have a desired biological activity on the cells, tissues, organs or organisms into which the nucleic acids and/or peptides have been introduced. The invention also provides compositions comprising the complexes of the invention and one or more additional components. Suitable such compositions, for example, include pharmaceutical compositions comprising one or more of the complexes of the invention and one or more pharmaceutically acceptable carriers, excipients or diluents therefor. The invention also provides methods for producing such complexes and compositions, and methods of using such complexes and compositions to deliver one or more nucleic acid molecules and/or one or more peptides to cells, tissues, organs or organisms, for example for therapeutic or prophylactic purposes. The invention also provides kits comprising the complexes and compositions of the invention, and optionally further comprising one or more additional components suitable for use in or with the complexes and compositions, and/or for carrying out the methods, of the present invention.

III. Polypeptides

As noted above, the compositions and complexes of the present invention comprise one or more peptides, polypeptides or proteins. In certain aspects of the invention, the peptides, polypeptides or proteins used in these complexes and compositions are peptides, polypeptides or proteins that are to be delivered to cells, tissues, organs or organisms for any suitable biological, therapeutic and/or prophylactic purpose. In certain other aspects of the invention, the peptides, polypeptides or proteins used in the complexes of the present invention are cellular delivery peptides, polypeptides or proteins, such as (but not limited to) those described and used in WIPO/PCT publication no. WO 00/58488, the disclosure of which is incorporated herein by reference in its entirety.

As used herein, the term “polypeptide” includes without limitation peptides (oligopeptides), proteins, and polypeptides. All of these are polymers of two or more amino acids joined by an amino bond. Generally, peptides comprise from 2 to about a amino acid residues, wherein “a” is any whole integer between 5 and 50, preferably between 10 and 30, and may be isolated from natural sources or more typically are synthesized in vitro. As used herein, the term “oligopeptide” may be used interchangeably and equivalently with the term “peptide” as defined above. As used herein, “polypeptides” generally comprise about b amino acids, wherein “b” is any whole integer between 25 and 50,000, preferably between 50 and 10,000, and more preferably between 50 and 1,000. The term “protein” encompasses polypeptides, as well as complexes of two or more covalently or non-covalently bonded polypeptides. Polypeptides and proteins are purified from their natural sources and/or are synthesized using recombinant DNA technology.

Peptides, polypeptides, proteins and protein complexes suitable for use in the complexes, compositions and methods of the present invention include any peptide, polypeptide, protein and protein complex, or portion thereof, that has a desired biological or physiological effect on the cells, tissues, organs and organisms to which the peptides, polypeptides, proteins and protein complexes are delivered. Non-limiting examples of such peptides, polypeptides, proteins and protein complexes include:

enzymes, e.g., kinases; peptidases/proteinases; oxidoreductases; nucleases;

recombinases (including Cre, Int, Flp, Tn5 resolvase, and the like); ligases (including DNA ligases and the like); lyases; isomerases (including topoisomerases and the like);

polymerases (including DNA polymerases, RNA polymerases, reverse transcriptases, and the like); transferases (including terminal transferases, glutathione S-transferases, and the like); ATPases; GTPases; etc.;

cytokines, e.g., growth factors (such as epidermal growth factor (EGF), fibroblast growth factors (FGFs), keratinocyte growth factors (KGFs), hepatocyte growth factors (HGFs), platelet-derived growth factor (PDGF), transforming growth factors alpha and beta (TGF-α and TGF-β), neurotrophic factor (NTF), ciliary neurotrophic factor (CNTF), brain-derived neurotrophic factor (BDNTF), glial-derived neurotrophic factor (GDNTF), bone morphogenic proteins (BMPs), and the like, and variants thereof); interleukins (such as IL-1 through IL-18, and the like, and variants thereof); interferons (such as IFN-α, IFN-β, IFN-γ, and the like, and variants thereof); colony-stimulating factors (such as granulocyte colony-stimulating factor (G-CSF), macrophage colony-stimulating factor (M-CSF), granulocyte-macrophase colony-stimulating factor (GM-CSF); erythropoietin (Epo); thrombopoietin (Tpo); leukemia inhibitory factor (LIF/Steel Factor); tumor-necrosis factors (TNFs); and the like, and variants thereof); peptide hormones (such as antidiuretic hormone, chorionic gonadotropin, leutenizing hormone, follicle-stimulating hormone, insulin, prolactin, somatomedins, growth hormone, thyroid-stimulating hormone, placental lactogen, and the like, and variants thereof); etc.;

intraceullar signalling peptides;

receptors (e.g., cytokine receptors, hormone receptors, antibody receptors, integrins and other extracellular matrix receptors, neurotransmitter receptors, viral receptors, and the like, and variants thereof);

antibodies (e.g., polyclonal or monoclonal antibodies, fragments thereof (including Fab and Fc fragments and portions thereof), and multi-antibody complexes);

vaccine components (including, but not limited to, proteins or peptides of etiologic agents such as viruses, bacteria, fungi (including yeasts), parasites and the like; proteins or peptides of tumor cells or other cancer-related proteins or peptides; and other proteins or peptides against which it is desirable to produce an immune response in an animal, suitably a mammal such as a human);

structural and/or functional proteins or peptides (e.g., hemoglobin, albumins including serum albumins, cytoskeletal proteins, transmembrane channel proteins or peptides, and the like, and fragments or variants thereof);

synthetic peptides (e.g., hexahistidine (His₆), polylysine, and other synthetic peptides of any length containing a desired sequence of two or more amino acids linked together by peptide bonds to form a peptide, oligopeptide, polypeptide or protein, any and all of which can be produced by art-known methods of synthetic peptide synthesis that will be familiar to the ordinarily skilled artisan, and that are described herein);

and the like. Of course, other suitable peptides, oligopeptides, polypeptides and proteins suitable for use in accordance with the present invention (i.e., in the complexes, compositions and methods of the invention) will be familiar to one of ordinary skill and therefore are encompassed by the present invention.

A. Amino Acids

The term “amino acid” as used herein refers generally to a molecule having both a carboxyl (—COOH) and an amino (—NH₂) group attached to the same carbon atom, called the alpha-carbon atom. Amino acids can be represented by the general formula R—CH(NH₂)COOH, wherein R is a side chain or residue which may or may not occur naturally. Generally, the side chain (R) of an amino acid contains c carbon atoms, d nitrogen atoms, 0, 1 or 2 sulfur atoms, d oxygens, and/or d halogen atoms, wherein “c” is any whole integer from 0 to about 20, and “d” is any whole integer from 0 to about 5.

The terms “natural amino acid” and “naturally-occurring amino acid” refer to Ala, Asp, Cys, Glu, Phe, Gly, His, Ile, Lys, Leu, Met, Asn, Pro, Gln, Arg, Ser, Thr, Val, Trp, and Tyr. “Unnatural amino acids” (i.e., amino acids do not occur naturally) include, by way of non-limiting example, homoserine, homoarginine, citrulline, phenylglycine, taurine, iodotyrosine, seleno-cysteine, norleucine (“Nle”), norvaline (“Nva”), beta-Alanine, L- or D-naphthalanine, omithine (“Orn”), and the like.

Amino acids also include the D-forms of natural and unnatural amino acids. “D-” designates an amino acid having the “D” (dextrorotary) configuration, as opposed to the configuration in the naturally occurring (“L-”) amino acids. Where no specific configuration is indicated, one skilled in the art would understand the amino acid to be an L-amino acid. The amino acids can, however, also be in racemic mixtures of the D- and L-configuration. Natural and unnatural amino acids can be purchased commercially (Sigma Chemical Co.; Advanced Chemtech) or synthesized using methods known in the art. Amino acid substitutions may be made on the basis of similarity in polarity, charge, solubility, hydrophobicity, hydrophilicity, and/or the amphipathic nature of the residues as long as their biological activity is retained.

B. Peptide Synthesis

Peptides used in accordance with the present invention may be produced by a variety of methods that will be familiar to those of ordinary skill in the art. For reviews and enabling disclosures of peptide synthesis, see M. Bodanzsky, “Principles of Peptide Synthesis,” 1st and 2nd revised ed., Springer-Verlag, New York, N.Y., 1984 and 1993; Stewart and Young, “Solid Phase Peptide Synthesis,” 2nd ed., Pierce Chemical Co., Rockford, Ill., 1984; Fox J E. Multiple peptide synthesis. Mol Biotechnol. 3:249-258, 1995; Kiso Y, Fujii N, Yajima H. New disulfide bond-forming reactions for peptide and protein synthesis. Braz J Med Biol Res. 27:2733-2744, 1994; Bongers J, Heimer E P. Recent applications of enzymatic peptide synthesis. Peptides. 15:183-193, 1994; Wade J D, Tregear G W. Solid phase peptide synthesis: recent advances and applications. Australas Biotechnol. 3:332-336, 1993; Fields G B, Noble R L. Solid phase peptide synthesis utilizing 9-fluorenylmethoxycarbonyl amino acids. Int J Pept Protein Res. 35:161-214, 1990; Newton R, Fox J E. Automation of peptide synthesis. Adv Biotechnol Processes. 10:1-24, 1988; Barany G, Kneib-Cordonier N, Mullen D G. Solid-phase peptide synthesis: a silver anniversary report. Int J Pept Protein Res. 30:705-739, 1987; Bodanszky M. In search of new methods in peptide synthesis. A review of the last three decades. Int J Pept Protein Res. 25:449-474, 1985; Chaiken I M. Semisynthetic peptides and proteins. CRC Crit Rev Biochem. 11:255-301, 1981; Fridkin M, Patchomnik A. Peptide synthesis. Annu Rev Biochem. 43:419-443, 1974; Merrifield R B. Solid-phase peptide synthesis. Adv Enzymol Relat Areas Mol Biol. 32:221-296, 1969; and U.S. Pat. No. 4,748,002 (Semi-automatic, solid-phase peptide multi-synthesizer and process for the production of synthetic peptides by the use of the multi-synthesizer) to Neimark et al.

C. Fusion Proteins

In certain embodiments, the peptides, polypeptides or proteins used in the present invention are in the form of fusion proteins. As used herein, the term “fusion protein” refers to a peptide, polypeptide or protein comprising a series of contiguous amino acids from one peptide, polypeptide or protein that are linked via peptide bonds to a series of contiguous amino acids from one or more additonal peptides, polypeptides or proteins. For example, fusion of the glutathione S-transferase (GST) domain to a peptide, polypeptide or protein of interest allows the fusion protein to be purified by affinity chromatography on glutathione agarose (Pharmacia, Inc., 1995 catalog). The fusion protein may include one or more accessory sequences which function for detection, purification or cleavage of the fusion protein. If the peptide, polypeptide or protein of interest is fused to a series of of consecutive histidines (for example 6×His), the fusion protein can be purified by affinity chromatography on chelating resins containing metal ions (Qiagen, Inc.). Fusion proteins may include sequences which function as a protein tag, such as an antibody epitope (e.g., dervied from Myc), a thiorescent peptide or a poly Histag. Tags and other elements may function in the purification and/or detection of the fusion protein. In producing fusion proteins according to this aspect of the invention, it is often desirable to compare amino terminal and carboxy terminal fusions for activity, solubility, stability, and the like.

Targeting sequences are another type of accessory element that can be comprised in a fusion protein. Cellular targeting elements, which direct fusion proteins to specific cell types, include such things as antibody fragments directed to a cellular surface molecule, fragments of ligands for receptors present on a cell, cell-specific targeting sequences derived from pathogens, derivatives of cellular adhesion molecules, and the like. Intracellular targeting elements, which direct fusion proteins to subcellular locations including, without limitation, the nucleus, the cell membrane, the chloroplast, the mitochondrion, the endoplasmic reticulum, the cytoplasm, and membranes or intermembrane spaces of any of the preceding, are known and are commercially available (e.g., Invitrogen's line of pShooter™ vectors). Various targeting sequences are known in the art and can be readily incorporated into fusion proteins using methods known in the art. Polynucleotides encoding fusion proteins may be constructed by standard molecular biology techniques (J. Sambrook, E. F. Fritsch and T. Maniatis (1989). Molecular Cloning, A Laboratory Manual. Cold Spring Harbor Laboratory Press. Cold Spring Harbor, N.Y.).

IV. Cellular Delivery Molecules

Non-limiting examples of cellular delivery molecules suitable for use in the compositions, complexes and methods of the present invention include translocating peptides and proteins, and peptide and protein analogs (peptoids), which are defined by their ability to cross biological membranes, and DNA-binding peptides, oligopeptides or polypeptides. Translocating peptides and proteins include, but are not limited to, those described and used in WIPO/PCT publication no. WO 00/58488, the disclosure of which is incorporated herein by reference in its entirety and peptoid analogs thereof.

1. Translocating Peptides and Proteins

When a translocating peptide or protein is applied to the medium of cultured mammalian cells, the peptide or protein is taken up and may accumulate in the cytoplasm or nucleus (or other organelle) of the cell. Translocating peptides and proteins that have been described include but are not limited to the VP22 protein and functional fragments thereof from Herpes Simplex Virus type 1 (Elliott, G., and O'Hare, P., Cell 88:223-233 (1997)), peptides derived from the HIV Tat protein, the Drosophila homeodomain protein Antennapedia (Derrossi et al., 1994, 1996) and fragments thereof or the Kaposi basic FGF receptor (K-FGF) (Rojas et al., 1998; Dokka, S., Pharm Res 14:1759-64 (1997)). Peptides which have the ability to penetrate cell membranes have been called “cell penetrating peptides” or “protein transduction sequences.” For reviews of translocating proteins and peptides, see Schwartz, J. J., and Zhang, S., Curr Opin Mol Ther. 2:162-167 (2000); and Schwarze, S. R., et al., Trends Cell Biol. 10:290-295 (2000); Schwarze, S. R. and Dowey, S. F., Trends Pharmacol. Sci.21:45-48 (2000); and Lindgren, M. et al. Trends Pharmacol. Sic. 21:99-113 (2000).

The biological functions of the VP22, Tat and Antennapedia proteins are distinct. VP22 is a structural protein found in the tegument region of HSV-1 and is essential for viral infectivity (Ellliot, G. D. and Meredith, J. Gen Virol. 73:723-726 (1992)). Tat is required for activation of expression from the HIV-1 long terminal repeat (reviewed by Cullen, B. R. and Green, W. C., Cell 58:423-426 (1989)). In contrast, Antennapedia is a transcriptional activator containing a homeodomain and is required for Drosophila development (reviewed by Gering, W. J., Science 236:1245-1252 (1987)).

Similarly, the amino acid sequences of these proteins that are involved in cellular uptake are distinct. Amino acids 159-301 of VP22 are sufficient for uptake by cultured mammalian cells, and uptake of VP22 is abolished by deletion of the C-terminal 34 residues. Smaller fragments of Tat (amino acids 46-60), Antennapedia (amino-acids 42-58) Antennapedia(43-58), Antennapedia(41-50), and KFGF (amino-acids 1-12) are sufficient for uptake by mammalian cells. A number of protein and peptide fusions with VP22(159-301), Tat(48-60), Antennapedia(43-58) and kFGF(1-12) have been described, including fusion proteins comprising a second polypeptide, i.e., green fluorescent protein (GFP), p53, thymidine kinase, p₂₇^{kiP1} caspase-3, β-galactosidase, members of the rab small GTPase family, the Grb2 SH2 domain and Cre recombinase. In each case, the biological activity of the second polypeptide has been demonstrated following intracellular delivery (Elliot and O'Hare, 1997, Dilber et al., 1999, Nagahra, et al., Vocero-Akbani et al., 1999, Schwarze, S. R., et al., Science 285:1569-1572 (1999), Rojas et al. 1999, Perez, F., et al., Mol. Endocrinol. 8:1278-1287 (1994), Rojas et al., 1998, Jo, D., et al., Nat. Biotechnol. 19:929-933 (2001)).

VP22, Antennapedia and Tat have some gross structural similarities, e.g., each protein has a region containing a number of lysine or arginine residues separated by uncharged residues. Secondary structure predictions indicate that these basic regions can form alpha-helices. Recently, a number of other membrane translocating peptide have been identified (Mi, Z., et al., Mol. Therapy 2:339-347 (2000); Suzuki, et al., 2001; Futaki et al., 2002; and Wender, P. A., et al., Proc. Natl Acad. Sci. USA. 97:13003-13008 (2000)), and the only similarity between these peptides is their high arginine content. Polyarginine peptides only six to eleven residues in length appear to have translocating activities similar to Tat(48-60) (Wender, P. A., et al., Proc. Natl Acad. Sci. USA. 97:13003-13008 (2000); Suzuki et al., 2001; Masayuki et al., 2001; and Han, K., et al., Mol. Cells 12:267-271 (2001)). Published U.S. patent application 20030032593 (Feb 13, 2003) describes translocating peptides having spaced arginine moieties.

The peptides are described as having structures selected from the group (ZYZ)ₙZ, (ZY)ₙZ, (ZYY)ₙA and (ZYYY)ₙZ, where Z is L-arginine or D-arginine, Y is an amino acid other than one that contains an amidino or guanidino moiety and n is an integer ranging from 2 to 10. This published application is incorporated by reference herein it its entirety and specifically for its description of the synthesis and application of the translocating peptides.

The description and synthesis of protein targeting domains, whether they are designed de novo or are derived from a naturally occurring protein such as VP22, Tat and Ant, is known. See, by way of non-limiting example, Dokka, S., Pharm Res 14:1759-64 (1997). Cellular delivery of oligonucleotides by synthetic import peptide carrier. Pharm Res 14, 1759-64; Futaki, S., Suzuki, et al., J. Biol. Chem. 276:5836-5840 (2001a). Arginine-rich peptides. An abundant source of membrane-permeable peptides having potential as carriers for intracellular protein delivery. J. Biol. Chem. 276:5836-5840; Futaki S., et al., Bioconjug Chem. 12:1005-1011 (2001b). Stearylated arginine-rich peptides: a new class of transfection systems. Bioconjug Chem. 12:1005-1011; Ho, et al., Cancer Research 61:474-477 (2001). Synthetic protein transduction domains: enhanced transduction potential in vitro and in vivo. Cancer Research 62, 474-477; Mi, Z., et al., Mol. Therapy 2:339-347 (2000), Mai, et al., (2002). Characterization of a Class of Cationic Peptides Able to Facilitate Efficient Protein Transduction in vitro and in vivo. Mol. Therapy 2, 339-347; Morris, M. C., et al., Nat Biotechnol 19:1173-6 (2001). A peptide carrier for the delivery of biologically active proteins into mammalian cells. Nat Biotechnol 19, 1173-6; Rothbard, J. B., et al., J Med Chem. 15:3612-3618 (2002). Arginine-rich molecular transporters for drug delivery: role of backbone spacing in cellular uptake. J Med Chem. 15, 3612-8; Wender, P. A., et al., Proc. Natl Acad. Sci. USA. 97:13003-13008 (2000)). The design, synthesis, and evaluation of molecules that enable or enhance cellular uptake: peptoid molecular transporters. Proc. Natl Acad. Sci. USA. 97, 13003-13008; Suzuki, T., et al., J. Biol. Chem. 277:2437-2443 (2002). Possible existence of common internalization mechanisms among arginine-rich peptides. J. Biol. Chem. 277, 2437-2443; and published PCT Patent Application WO 02/065986, Transporters comprising spaced arginine moieties, to Wender et al.

1A. Peptoid Analogs of Translocating Peptides

Peptoid analogs of certain translocating peptides have been shown to function for translocation across cell membranes. For example, a series of polyguanidine peptoid derivatives (N-argX, where x is 5-9) were designed as peptidomimetic analogs of Tat49-57 and were demonstrated to be taken up by cells in amounts only slightly lower than R5, R7 and R9 (Wender, P. A. et al. Proc. Natl. Acad. Sci. 97:13003-13008 (2000)). This reference discloses polyguanidine peptoids having general structure:

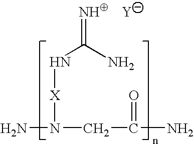

where X is (CH₂)ₘ, m is 2, 3, 4, 6 or 8, and Ye is an anion, e.g. CF3CO₂, which function for translocation into cells. Peptoids of this formula where N is 9 and m is 4-8 are particularly useful for translocation. Methods for synthesis of peptoids are known in the Wendu et al. 2000 supra and references therein provide useful synthetic methods.

2. DNA-Binding Peptides and Proteins

A variety of DNA-binding proteins, particularly those that are basic, more particularly DNA-binding proteins with a relatively high percentage of Lysine and Arginine residues (“Arg- and Lys-rich proteins”), can be used to practice the invention. A DNA-binding protein can be sequence-specific, partially sequence specific, or non-specific. Non-limiting examples of DNA-binding peptides and proteins suitable for use in the present invention are detailed in the following subsections.

a. Polylysine and Other Cationic Homopolypeptides

Polylysine (“poly-Lys”) is known in the art to complex and compact nucleic acids. Studies by Olins and von Hipple (J. Mol. Bio. 24:157-176, 1967) using cationic homopolypeptides as models for nucleoprotein complex formation suggested that complexes of DNA with cationic polypeptides (including without limitation poly-lysine) form after “annealing” both components in solution, i.e., by step-down dialysis from NaCl concentrations of 2 M to 0.010 M. Studies of nucleic acid compaction by poly-Lys are ongoing (Laurent et al., 1999. Uptake by rat liver and intracellular fate of plasmid DNA complexed with poly-L-lysine or poly-D-lysine. FEBS Lett 443:61-65. Molas et al., 2002. Single-stranded DNA condensed with poly-L-lysine results in nanometric particles that are significantly smaller, more stable in physiological ionic strength fluids and afford higher efficiency of gene delivery than their double-stranded counterparts. Biochim Biophys Acta 1572:37-44; and Schwarzenberger et al., 2001. Poly-L-lysine-based molecular conjugate vectors: a high efficiency gene transfer system for human progenitor and leukemia cells. Am J Med Sci 321:129-136).

In addition to poly-Lys per se, various chemically modified derivatives of poly-Lys have been used. These include without limitation:

lactosylated poly-Lys (Erbacher et al., 1996. Putative role of chloroquine in gene transfer into a human hepatoma cell line by DNA/lactosylated polylysine complexes. Exp Cell Res 225:186-194; Kollen et al., 1999. Enhanced efficiency of lactosylated poly-L-lysine-mediated gene transfer into cystic fibrosis airway epithelial cells. Am J Respir Cell Mol Biol 20:1081-1086; and Klink et al., 2001. Nuclear translocation of lactosylated poly-L-lysine/cDNA complex in cystic fibrosis airway epithelial cells. Mol Ther 3:831-841);

galactosylated poly-Lys (Han J, Il Yeom Y., 2000. Specific gene transfer mediated by galactosylated poly-L-lysine into hepatoma cells. Int J Pharm 202:151-160; and Hashida et al., 1998. Targeted delivery of plasmid DNA complexed with galactosylated poly(L-lysine). J Control Release 53:301-310);

histidylated poly-Lys (Aoki et al., 2001. Potential tumor-targeting peptide vector of histidylated oligolysine conjugated to a tumor-homing RGD motif. Cancer Gene Ther 8:783-787; Midoux P, Monsigny M., 1999. Efficient gene transfer by histidylated polylysine/pDNA complexes. Bioconjug Chem 10:406-411; and Bello et al, 2001. Histidylated polylysine as DNA vector: elevation of the imidazole protonation and reduced cellular uptake without change in the polyfection efficiency of serum stabilized negative polyplexes. Bioconjug Chem 12:92-99);

poly-Lys conjugated with hydrophilic polymers, such as, by way of non-limiting example, polyethylene glycol (PEG) and derivatized PEG moieties (Toncheva et al., 1998. Novel vectors for gene delivery formed by self-assembly of DNA with poly(L-lysine) grafted with hydrophilic polymers. Biochim Biophys Acta 1380:354-368; Lee et al., 2002. PEG grafted polylysine with fusogenic peptide for gene delivery: high transfection efficiency with low cytotoxicity. J Control Release 79:283-291; Choi et al., 1998. Polyethylene glycol-grafted poly-L-lysine as polymeric gene carrier. J Control Release 54:39-48; Nah et al., 2002. Artery wall binding peptide-poly(ethylene glycol)-grafted-poly(L-lysine)-based gene delivery to artery wall cells. J Control Release 78:273-284; and Choi et al., 1999. Characterization of a targeted gene carrier, lactose-polyethylene glycol-grafted poly-L-lysine and its complex with plasmid DNA. Hum Gene Ther 10:2657-2665);

poly-Lys conjugated with folic acid (Ginobbi et al., 1997. Folic acid-polylysine carrier improves efficacy of c-myc antisense oligodeoxynucleotides on human melanoma (M14) cells. Anticancer Res 17:29-35);

poly-Lys conjugated with disulfide-containing cationic polymers, which allow for the intracellular release of nucleic acid in a reductive medium, including without limitation Poly[Lys-(AEDTP) (Pichon et al., 2002. Poly[Lys-(AEDTP)]: a cationic polymer that allows dissociation of pDNA/cationic polymer complexes in a reductive medium and enhances polyfection. Bioconjug Chem 13:76-82); and

gluconoylated poly-Lys (Erbacher et al., 1997. The reduction of the positive charges of polylysine by partial gluconoylation increases the transfection efficiency of polylysine/DNA complexes. Biochim Biophys Acta 1324:27-36).

See U.S. Pat. No. 5,354,844 (Protein-polycation conjugates) to Beug, et al.; U.S. Pat. No. 5,972,900 (Delivery of nucleic acid to cells) to Ferkol, Jr., et al.; U.S. Pat. No. 5,166,320 (Carrier system and method for the introduction of genes into mammalian cells); and U.S. Pat. Nos. 6,008,336, 5,844,107 and 5,877,302 (Compacted nucleic acids and their delivery to cells), U.S. Pat. No. 6,077,835 (Delivery of compacted nucleic acid to cells), all to Hanson, et al. U.S. Pat. No. 6,333,396 to Filpula, et al. (Method for targeted delivery of nucleic acids) describes a single-chain antigen-binding polypeptide comprising, at its C-terminus, N-terminus, or both, basic amino acid residues selected from the group consisting of oligo-Lys, oligo-Arg and combinations thereof. U.S. Pat. No. 6,281,005 (Automated nucleic acid compaction device) to Hanson, et al. describes a device that can be used to prepare compacted DNA complexes.

b. Non-Eukaryotic Histonelike Proteins

One class of DNA-binding, Arg- and Lys-rich proteins that can be used in the invention is any non-eukaryotic histonelike protein. By way of non-limiting example, these include HU protein and IHF (integration host factor). HU and IHF proteins have been identified and cloned from a variety of eubacteria and archaea, including by way of non-limiting example Aeromonas proteolytica, Bacillus caldolyticus, Bacillus caldotenax, Bacillus cereus, Bacillus globigii, Bacillus stearothermophilus, Bacillus subtilis, Bifidobacterium longum, Borrelia burgdorferi, Campylobacter jejuni, Escherichia coli, Mycoplasma gallisepticum, Neisseria gonorrhoeae, Pseudomonas aeruginosa, Pseudomonas putida, Rhodobacter capsulatus, Salmonella typhimurium, Serratia marcescens, and Thermotoga maritima.

Exemplary non-eukaryotic histonelike proteins suitable for use in accordance with the present invention include, but are not limited to, those described in Table 3.

TABLE 3
Non-limiting Examples of Non-Eukaryotic Histone-like Proteins

| | | |
|---|---|---|
| | SOURCE | |
| PROTEIN | ORGANISM | REFERENCE(S) |

HU Proteins

| | | |
|---|---|---|
| | Aeromonas | Giladi H, Wang W X, Oppenheim A B. Isolation and characterization of the hupA gene coding for HU of |
| | proteolytica | Aeromonas proteolytica. Nucleic Acids Res. 1992 Aug 11; 20(15): 4092. |
| | Borrelia | Tilly K, Fuhrman J, Campbell J, Samuels D S. Isolation of Borrelia burgdorferi genes encoding |
| | burgdorferi | homologues of DNA-binding protein HU and ribosomal protein S20. Microbiology. 1996 Sep; 142 (Pt |
| | | 9): 2471-9. |
| HBsu | Bacillus | Kawamura S, Kajiyama H, Yamasaki N, Kimura M. Cloning of the gene encoding DNA binding protein |
| | stearothermophilus | HU from Bacillus stearothermophilus and its expression in Escherichia coli. Biosci Biotechnol Biochem. |
| | | 1995 Jan; 59(1): 126-9. |
| | | Groch N, Hahn U, Heinemann U. Synthesis of the Bacillus subtilis histone-like DNA-binding protein HBsu |
| | | in Escherichia coli and secretion into the periplasm. Gene. 1993 Feb 14; 124(1): 99-103. |
| | Bacillus | Padas P M, Wilson K S, Vorgias C E. Gene 1992 Aug 1; 117(1): 39-44. The DNA-binding protein HU from |
| | caldolyticus | mesophilic and thermophilic bacilli: gene cloning, overproduction and purification. |
| | Bacillus caldotenax | Padas et al., 1992. |
| | Bacillus subtilis | Padas et al., 1992. |
| | Bacillus globigii | Padas et al., 1992. |
| | Bifidobacterium | Takeuchi A, Matsumura H, Kano Y. Cloning and expression in Escherichia coli of a gene, hup, encoding |
| | longum | the histone-like protein HU of Bifidobacterium longum. Biosci Biotechnol Biochem. 2002 Mar; 66(3): 598-603. |
| | Campylobacter | Konkel M E, Marconi R T, Mead D J, Cieplak W Jr. Cloning and expression of the hup encoding a histone- |
| | jejuni | like protein of Campylobacter jejuni. Gene. 1994 Aug 19; 146(1): 83-6. |
| | Chlamydia | Zhong J, Douglas A L, Hatch T P. Characterization of integration host factor (IHF) binding upstream of the |
| | trachomatis | cysteine-rich protein operon (omcAB) promoter of Chlamydia trachomatis LGV serovar L2. Mol |
| | | Microbiol. 2001 Jul; 41(2): 451-62. |
| HU | Escherichia coli | Oberto J, Drlica K, Rouviere-Yaniv J. Histones, HMG, HU, IHF: Meme combat. Biochimie. 1994; 76(10-11): |
| | | 901-8. (review) |
| | Mycoplasma | Kenri T, Sasaki T, Kano Y. Identification and characterization of HU protein from Mycoplasma |
| | gallisepticum | gallisepticum. Biochem Biophys Res Commun. 1998 Aug 10; 249(1): 48-52. |
| | Pseudomonas | Delic-Attree I, Toussaint B, Vignais P M. Cloning and sequence analyses of the genes coding for the |
| | aeruginosa | integration host factor (IHF) and HU proteins of Pseudomonas aeruginosa. Gene. 1995 Feb 27; 154(1): 61-4. |
| | Salmonella | Higgins N P, Hillyard D. Primary structure and mapping of the hupA gene of Salmonella typhimurium. J |
| | typhimurium | Bacteriol. 1988 Dec; 170(12): 5751-8. |
| | Serratia | Oberto J, Rouviere-Yaniv J. Serratia marcescens contains a heterodimeric HU protein like Escherichia coli |
| | marcescens | and Salmonella typhimurium. J Bacteriol. 1996 Jan; 178(1): 293-7. |
| HSth | Streptococcus | Dixon-Fyle S M, Caro L. Characterization in vitro and in vivo of a new HU family protein from |
| | thermophilus | Streptococcus thermophilus ST11. Plasmid 1999 Nov; 42(3): 159-73. |
| | Thermotoga | Esser D, Rudolph R, Jaenicke R, Bohm G. The HU protein from Thermotoga maritima: recombinant |
| | maritima | expression, purification and physicochemical characterization of an extremely hyperthermophilic DNA- |
| | | binding protein. J Mol Biol. 1999 Sep 3; 291(5): 1135-46. |
| | | Christodoulou E, Vorgias C E. Cloning, overproduction, purification and crystallization of the DNA |
| | | binding protein HU from the hyperthermophilic eubacterium Thermotoga maritima. Acta Crystallogr D |
| | | Biol Crystallogr. 1998 Sep 1; 54 (Pt 5): 1043-5. |

IHF Proteins

| | | |
|---|---|---|
| | Brucella abortus | Microbiology 2000 Feb; 146 (Pt 2): 487-95. The genes for erythritol catabolism are organized as an |
| | | inducible operon in Brucella abortus. Sangari F J, Aguero J, Garcia-Lobo J M. |
| | Caulobacter | Gober J W, Shapiro L. A developmentally regulated Caulobacter flagellar promoter is activated by 3′ |
| | crescentus | enhancer and IHF binding elements. Mol Biol Cell. 1992 Aug; 3(8): 913-26. |
| IHF | Escherichia coli | Rice P A. Making DNA do a U-turn: IHF and related proteins. Curr Opin Struct Biol 1997 Feb; 7(1): 86-93. |
| | | (review) |
| | Neisseria | Hill S A, Samuels D S, Carlson J H, Wilson J, Hogan D, Lubke L, Belland R J. Integration host factor is a |
| | gonorrhoeae | transcriptional cofactor of pilE in Neisseria gonorrhoeae. Mol Microbiol. 1997 Feb; 23(4): 649-56. |
| | Pseudomonas | Delic-Attree I, Toussaint B, Vignais P M. Cloning and sequence analyses of the genes coding for the |
| | aeruginosa | integration host factor (IHF) and HU proteins of Pseudomonas aeruginosa. Gene. 1995 Feb 27; 154(1): 61-4. |
| | Pseudomonas | Calb R, Davidovitch A, Koby S, Giladi H, Goldenberg D, Margalit H, Holtel A, Timmis K, Sanchez-Romero J M, |
| | putida | de Lorenzo V, Oppenheim A B. Structure and function of the Pseudomonas putida integration |
| | | host factor. J Bacteriol. 1996 Nov; 178(21): 6319-26. |
| | | Toussaint B, David L, de Sury d'Aspremont R, Vignais P M. The IHF proteins of Rhodobacter capsulatus |
| | | and Pseudomonas aeruginosa. Biochimie. 1994; 76(10-11): 951-7. |
| | Pseudomonas | Valls M, Buckle M, de Lorenzo V. In vivo UV laser footprinting of the Pseudomonas putidasigma 54Pu |
| | putidasigma | promoter reveals that integration host factor couples transcriptional activity to growth phase. J Biol Chem. |
| | | 2002 Jan 18; 277(3): 2169-75. |
| | Rhizobium | Sojda J 3rd, Gu B, Lee J, Hoover T R, Nixon B T. A rhizobial homolog of IHF stimulates transcription of |
| | leguminosarum | dctA in Rhizobium leguminosarum but not in Sinorhizobium meliloti. Gene. 1999 Oct 1; 238(2): 489-500. |
| | Rhodobacter | Toussaint B, Delic-Attree I, De Sury D'Aspremont R, David L, Vincon M, Vignais P M. Purification of the |
| | capsulatus | integration host factor homolog of Rhodobacter capsulatus: cloning and sequencing of the hip gene, which |
| | | encodes the beta subunit. J Bacteriol. 1993 Oct; 175(20): 6499-504. |
| | | Toussaint B, Bosc C, Richaud P, Colbeau A, Vignais P M. A mutation in a Rhodobacter capsulatus gene |
| | | encoding an integration host factor-like protein impairs in vivo hydrogenase expression. Proc Natl Acad Sci |
| | | U S A. 1991 Dec 1; 88(23): 10749-53. |
| | | Toussaint B, David L, de Sury d'Aspremont R, Vignais P M. The IHF proteins of Rhodobacter capsulatus |
| | | and Pseudomonas aeruginosa. Biochimie. 1994; 76(10-11): 951-7. |

HU/IHF Family of Proteins (not classified as either)

| | | |
|---|---|---|
| Hbb Protein | Borrelia | Tilly K, Fuhrman J, Campbell J, Samuels D S. Isolation of Borrelia burgdorferi genes encoding homologues |
| | burgdorferi | of DNA-binding protein HU and ribosomal protein S20. Microbiology. 1996 Sep; 142(Pt 9): 2471-9. |
| Hbb Protein | B. burgdorferi | Valsangiacomo C, Balmelli T, Piffaretti J C. A nested polymerase chain reaction for the detection of |
| | sensu lato | Borrelia burgdorferi sensu lato based on a multiple sequence analysis of the hbb gene. FEMS Microbiol |
| | | Lett. 1996 Feb 1; 136(1): 25-9. |
| | | Valsangiacomo C, Balmelli T, Piffaretti J C. A phylogenetic analysis of Borrelia burgdorferi sensu lato |
| | | based on sequence information from the hbb gene, coding for a histone-like protein. Int J Syst Bacteriol. |
| | | 1997 Jan; 47(1): 1-10. |
| Hbb Protein | Borrelia turicatae | Valsangiacomo et al., 1996; Valsangiacomo et al., 1997 |
| Hbb Protein | Borrelia parkeri | Valsangiacomo et al., 1996; Valsangiacomo et al., 1997 |

| | |
|---|---|
| HU/IHF Hybrid Proteins | Goldenberg D, Giladi H, Oppenheim A B. Genetic and biochemical analysis of IHF/HU hybrid proteins. |
| | Biochimie. 1994; 76(10-11): 941-50. |
| Review | Christodoulou E, Vorgias C E, The thermostability of DNA-binding protein HU from mesophilic, |
| | thermophilic, and extreme thermophilic bacteria, Extremophiles 2002 Feb; 6(1): 21-31. |

C. Histones

Another class of DNA-binding, Arg- and Lys-rich protein that can be used in the complexes and compositions of the present invention is a histone or mixture of a histones. Any histone protein, including without limitation H1, H2A, H2B, H3 and H4, can be used.

The use of histone proteins to mediate or enhance transfection is described in the following references, all of which are incorporated herein by reference in their entireties: Balicki D, Beutler E. 1997. Histone H2A significantly enhances in vitro DNA transfection. Mol Med. 3:782-787; Balicki et al. 2000. Histone H2A-mediated transient cytokine gene delivery induces efficient antitumor responses in murine neuroblastoma. Proc Natl Acad Sci USA 97:11500-11504; Balicki et al. 2002. Structure and function correlation in histone H2A peptide-mediated gene transfer. Proc Natl Acad Sci USA 99:7467-7471;

Demirhan et al. 1998. Histone-mediated transfer and expression of the HIV-1 tat gene in Jurkat cells. J Hum Virol. 1:430-440; and Zaitsev et al. 2002. Histone HI-mediated transfection: role of calcium in the cellular uptake and intracellular fate of H1-DNA complexes. Acta Histochem 104:85-92. See also U.S. Pat. Nos. 6,180,784 and 5,744,335 (both entitled “Process of transfecting a cell with a polynucleotide mixed with an amphipathic compound and a DNA-binding protein”), both to Wolff, et al.; U.S. Pat. No. 6,458,382 (“Nucleic acid transfer complexes”) to Herweijer, et al.; published PCT application WO 96/14424 (“DNA transfer method”) to Hallybone; and published PCT application WO 99/19502, EP 0 967 288 A1, and EP 0 908 521 A1 (all entitled “Transfection System for the transfer of nucleic acids into cells”), all to Chandra, et al.

The human histone-like protein described in U.S. Pat. Nos. 5,851,799, 5,981,221 and 5,908,831 (all entitled “Histone-like protein), all to Bandman, et al., and the protein and peptide sequences described in U.S. Pat. Nos. 5,945,400 and 6,200,956, and Published PCT application WO 96/25508 (all entitled “Nucleic acid-containing composition, preparation and use thereof”), all to Scherman, et al., can also be used to practice the invention. Chemically modified histone proteins, including by way of non-limiting example galactosylated histones (Chen, et al., Hum Gene Ther 5:429-435, 1994), can be used in the invention. Histone proteins can be labeled with fluorophores using techniques known in the art. For example, Zaitsev et al. (2002) describe FITC-labeled histone H1. Moreover, histones can be used in combination with other transfection agents, such as the lipid DOSPER (Kott et al., 1998, A new efficient method for transfection of neonatal cardiomyocytes using histone H1 in combination with DOSPER liposomal transfection reagent. Somat. Cell Molec. Genet. 24:257-261).

V. Nucleic Acids

As noted above, the complexes of the present invention comprise one or more nucleic acids or nucleic acid molecules, which often will comprise one or more genes of interest, that can be delivered to cells, tissues, organs or organisms using the compositions, complexes and methods of the present invention. As used herein, the term “nucleic acids” (which is used herein interchangeably and equivalently with the term “nucleic acid molecules”) refers to nucleic acids (including DNA, RNA, and DNA-RNA hybrid molecules) that are isolated from a natural source; that are prepared in vitro, using techniques such as PCR amplification or chemical synthesis; that are prepared in vivo, e.g., via recombinant DNA technology; or that are prepared or obtained by any appropriate method. Nucleic acids used in accordance with the invention may be of any shape (linear, circular, etc.) or topology (single-stranded, double-stranded, linear, circular, supercoiled, torsional, nicked, etc.). The term “nucleic acids” also includes without limitation nucleic acid derivatives such as peptide nucleic acids (PNAS) and polypeptide-nucleic acid conjugates; nucleic acids having at least one chemically modified sugar residue, backbone, internucleotide linkage, base, nucleotide, nucleoside, or nucleotide analog or derivative; as well as nucleic acids having chemically modified 5′ or 3′ ends; and nucleic acids having two or more of such modifications. Not all linkages in a nucleic acid need to be identical.

Examples of nucleic acids include without limitation oligonucleotides (including but not limited to antisense oligonucleotides, ribozymes and oligonucleotides useful in RNA interference (RNAi)), aptamers, polynucleotides, artificial chromosomes, cloning vectors and constructs, expression vectors and constructs, gene therapy vectors and constructs, rRNA, tRNA, mRNA, mtRNA, and tmRNA, and the like. For reviews of the latter type of nucleic acid, see Muto A, Ushida C, Himeno H. A bacterial RNA that functions as both a tRNA and an mRNA. Trends Biochem Sci. 23:25-29, 1998; and Gillet R, Felden B. Emerging views on tmRNA-mediated protein tagging and ribosome rescue. Mol Microbiol. 42:879-885, 2001.

A. Oligonucleotides

As used in the present invention, an oligonucleotide is a synthetic or biologically produced molecule comprising a covalently linked sequence of nucleotides which may be joined by a phosphodiester bond between the 3′ position of the pentose of one nucleotide and the 5′ position of the pentose of the adjacent nucleotide. As used herein, the term “oligonucleotide” includes natural nucleic acid molecules (i.e., DNA and RNA) as well as non-natural or derivative molecules such as peptide nucleic acids, phophothioate-containing nucleic acids, phosphonate-containing nucleic acids and the like. In addition, oligonucleotides of the present invention may contain modified or non-naturally occurring sugar residues (e.g., arabinose) and/or modified base residues. The term oligonucleotide encompasses derivative molecules such as nucleic acid molecules comprising various natural nucleotides, derivative nucleotides, modified nucleotides or combinations thereof. Oligonucleotides of the present invention may also comprise blocking groups which prevent the interaction of the molecule with particular proteins, enzymes or substrates.

Oligonucleotides include without limitation RNA, DNA and hybrid RNA-DNA molecules having sequences that have minimum lengths of e nucleotides, wherein “e” is any whole integer from about 2 to about 15, and maximum lengths of about f nucleotides, wherein ‘f’ is any whole integer from about 2 to about 200. In general, a minimum of about 6 nucleotides, preferably about 10, and more preferably about 12 to about 15 nucleotides, is desirable to effect specific binding to a complementary nucleic acid strand.

In general, oligonucleotides may be single-stranded (ss) or double-stranded (ds) DNA or RNA, or conjugates (e.g., RNA molecules having 5′ and 3′ DNA “clamps”) or hybrids (e.g., RNA:DNA paired molecules), or derivatives (chemically modified forms thereof). Single-stranded DNA is often preferred, as DNA is less susceptible to nuclease degradation than RNA. Similarly, chemical modifications that enhance the specificity or stability of an oligonucleotide are preferred in some applications of the invention.

Certain types of oligonucleotides are of particular utility in the compositions and complexes of the present invention, including but not limited to antisense oligonucleotides, ribozymes, interfering RNAs and aptamers.

1. Antisense Oligonucleotides

Nucleic acid molecules suitable for use in the present invention include antisense oligonucleotides. In general, antisense oligonucleotides comprise nucleotide sequences sufficient in identity and number to effect specific hybridization with a preselected nucleic acid. Antisense oligonucleotides are generally designed to bind either directly to mRNA transcribed from, or to a selected DNA portion of, a targeted gene, thereby modulating the amount of protein translated from the mRNA or the amount of mRNA transcribed from the gene, respectively. Antisense oligonucleotides may be used as research tools, diagnostic aids, and therapeutic agents.

Antisense oligonucleotides used in accordance with the present invention typically have sequences that are selected to be sufficiently complementary to the target mRNA sequence so that the antisense oligonucleotide forms a stable hybrid with the mRNA and inhibits the translation of the mRNA sequence, preferably under physiological conditions. It is preferred but not necessary that the antisense oligonucleotide be 100% complementary to a portion of the target gene sequence. However, the presnt invention also encompasses the production and use of antisense oligonucleotides with a different level of complementarity to the target gene sequence, e.g., antisense oligonucleotides that are at least about 50% complementary, at least about 55% complementary, at least about 60% complementary, at least about 65% complementary, at least about 70% complementary, at least about 75% complementary, at least about 80% complementary, at least about 85% complementary, at least about 90% complementary, at least about 91% complementary, at least about 92% complementary, at least about 93% complementary, at least about 94% complementary, at least about 95% complementary, at least about 96% complementary, at least about 97% complementary, at least about 98% complementary, or at least about 99% complementary, to the target gene sequence. In certain embodiments, the antisense oligonucleotide hybridizes to an isolated target mRNA under the following conditions: blots are first incubated in prehybridization solution (5×SSC; 25 mM NaPO₄, pH 6.5; 1× Denhardt's solution; and 1% SDS) at 42° C. for at least 2 hours, and then hybridized with radiolabelled cDNA probes or oligonucleotide probes (1×10⁶ cpm/ml of hybridization solution) in hybridization buffer (5× SSC; 25 mM NaPO₄, pH 6.5; 1× Denhardt's solution; 250 ug/ml total RNA; 50% deionized formamide; 1% SDS; and 10% dextran sulfate). Hybridization for 18 hours at 30-42° C. is followed by washing of the filter in 0.1-6×SSC, 0.1% SDS three times at 25-55° C. The hybridization temperatures and stringency of the wash will be determined by the percentage of the GC content of the oligonucleotides in accord with the guidelines described by Sambrook et al. (Molecular Cloning: A Laboratory Manual, 2^{nd} edition, 1989, Cold Spring Harbor Laboratory Press, Plainview, N.Y.), including but not limited to Table 11.2 therein.

Representative teachings regarding the synthesis, design, selection and use of antisense oligonucleotides include without limitation U.S. Pat. No. 5,789,573, Antisense Inhibition of ICAM-1, E-Selectin, and CMV IE1/IE2, to Baker et al.; U.S. Pat. No. 6,197,584, Antisense Modulation of CD40 Expression, to Bennett et al.; and Ellington, 1992, Current Protocols in Molecular Biology, 2^{nd} Ed., Ausubel et al., eds., Wiley Interscience, New York, Units 2.11 and 2.12.

2. Ribozymes

Nucleic acid molecules suitable for use in the present invention also include ribozymes. In general, ribozymes are RNA molecules having enzymatic activities usually associated with cleavage, splicing or ligation of nucleic acid sequences. The typical substrates for ribozymes are RNA molecules, although ribozymes may catalyze reactions in which DNA molecules (or maybe even proteins) serve as substrates. Two distinct regions can be identified in a ribozyme: the binding region which gives the ribozyme its specificity through hybridization to a specific nucleic acid sequence (and possibly also to specific proteins), and a catalytic region which gives the ribozyme the activity of cleavage, ligation or splicing. Ribozymes which are active intracellularly work in cis, catalyzing only a single turnover, and are usually self-modified during the reaction. However, ribozymes can be engineered to act in trans, in a truly catalytic manner, with a turnover greater than one and without being self-modified. Owing to the catalytic nature of the ribozyme, a single ribozyme molecule cleaves many molecules of target RNA and therefore therapeutic activity is achieved in relatively lower concentrations than those required in an antisense treatment (WO 96/23569).

Representative teachings regarding the synthesis, design, selection and use of ribozymes include without limitation U.S. Pat. No. 4,987,071, RNA ribozyme polymerases, dephosphorylases, restriction endoribonucleases and methods, to Cech et al.; and U.S. Pat. No. 5,877,021, B7-1 Targeted Ribozymes, to Stinchcomb et al.; the disclosures of all of which are incorporated herein by reference in their entireties.

3. Nucleic Acids for RNAi (RNAi Molecules)

Nucleic acid molecules suitable for use in the present invention also include nucleic acid molecules, particularly oligonucleotides, useful in RNA interference (RNAi). In general, RNAi is one method for analyzing gene function in a sequence-specific manner. For reviews, see Tuschl, T., Chembiochem. 2:239-245 (2001), and Cullen, B. R., Nat Immunol. 3:597-599 (2002). RNA-mediated gene-specific silencing has been described in a variety of model organisms, including nematodes (Parrish, S., et al., Mol Cell 6:1077-1087 (2000); Tabara, H., et al., Cell 99:123-132 (1999); in plants, i.e., “co-suppression” (Napoli, C., et al., Plant Cell 2:279-289 (1990)) and post-transcriptional or homologous gene silencing (Hamilton, A. J. and D. C. Baulcombe, Science 286:950-952 (1999); Hamilton, et al., EMBO J 21:4671-4679 (2002)) (PTGS or HGS, respectively) in plants; and in fungi, i.e., “quelling” (Romano, N. and G. Macino, Mol Microbiol 6:3343-3353 (1992)). Examples of suitable interfering RNAs include siRNAs, shRNAs and stRNAs. As one of ordinary skill will readily appreciate, however, other RNA molecules having analogous interfering effects are also suitable for use in accrodance with this aspect of the present invention.

a. Small Interfering RNA (siRNA)

RNAi is mediated by double stranded RNA (dsRNA) molecules that have sequence-specific homology to their “target” mRNAs (Caplen, N. J., et al., Proc Natl Acad Sci USA 98:9742-9747 (2001)). Biochemical studies in Drosophila cell-free lysates indicates that the mediators of RNA-dependent gene silencing are 21-25 nucleotide “small interfering” RNA duplexes (siRNAs). Accordingly, siRNA molecules are advantageously used in the compositions, complexes and methods of the present invention. The siRNAs are derived from the processing of dsRNA by an RNase known as Dicer (Bernstein, E., et al., Nature 409:363-366 (2001)). It appears that siRNA duplex products are recruited into a multi-protein siRNA complex termed RISC (RNA Induced Silencing Complex). Without wishing to be bound by any particular theory, it is believed that a RISC is guided to a target mRNA, where the siRNA duplex interacts sequence-specifically to mediate cleavage in a catalytic fashion (Bernstein, E., et al., Nature 409:363-366 (2001); Boutla, A., et al., Curr Biol 11:1776-1780 (2001); Hammond et al., 2000).

RNAi has been used to analyze gene function and to identify essential genes in mammalian cells (Elbashir, et al., Methods 26:199-213 (2002); Harborth, et al., J Cell Sci 114:4557-4565 (2001)), including by way of non-limiting example neurons (Krichevsky, A. M. and Kosik, K. S., Proc Natl Acad Sci USA 99:11926-11929 (2002)). RNAi is also being evaluated for therapeutic modalities, such as inhibiting or block the infection, replication and/or growth of viruses, including without limitation poliovirus (Gitlin, et al, Nature 418:379-380 (2002)) and HIV (Capodici, et al., J Immunol 169:5196-5201 (2002)), and reducing expression of oncogenes (e.g., the bcr-abl gene; Scherr, et al., Blood September 26 (epub ahead of print) (2002)). RNAi has been used to modulate gene expression in mammalian (mouse) and amphibian (Xenopus) embryos (Calegari, et al., Proc Natl Acad Sci USA 99:14236-14240 (2002), and Zhou, et al., Nucleic Acids Res 30:1664-1669 (2002), respectively), and in postnatal mice (Lewis, et al., Nat Genet 32:107-108 (2002)), and to reduce trangsene expression in adult transgenic mice (McCaffrey, et al., Nature 418:38-39 (2002)).

Molecules that mediate RNAi, including without limitation siRNA, can be produced in vitro by chemical synthesis (Hohjoh, H., FEBS Lett 521:195-199 (2002)), hydrolysis of dsRNA (Yang, et al., Proc Natl Acad Sci USA 99:9942-9947 (2002)), by in vitro transcription with T7 RNA polymerase (Donze, 0. and Picard, D., Nucleic Acids Res 30:e46. (2002); Yu, et al., Proc Natl Acad Sci USA 99:6047-6052 (2002)), and by hydrolysis of double-stranded RNA using a nuclease such as E. coli RNase III (Yang, et al., Proc Natl Acad Sci USA 99:9942-9947 (2002)). RNAi molecules can also be expressed inside cells by endogenous RNA polymerases, using for example RNA Pol III which acts on the U6 RNA promoter (Yu, et al., Proc Natl Acad Sci USA 99:6047-6052 (2002); Paul, et al., Nat Biotechnol 20:505-508 (2002)). For example, the commercially available GeneSuppressor™ System (IMGENEX, San Diego, Calif.) uses vectors comprising the U6 promoter to generate RNAi molecules in vivo. Viral vectors for siRNA (Xia, et al., Nat Biotechnol 20:1006-1010 (2002)) including, by way of non-limiting example, retroviruses (Devroe, E. and Silver, P. A., BMC Biotechnol 2:15 (2002)), have also been described. Methods have been described for determining the efficacy and specificity of siRNAs in cell culture and in vivo (Bertrand, et al., Biochem Biophys Res Commun 296:1000-1004 (2002); Lassus, et al., Sci STKE 2002(147):PL13 (2002); Leirdal, M. and Sioud, M., Biochem Biophys Res Commun 295:744-748 (2002)).

Because the Dicer RNase facilitates siRNA production, it is expected that cells that express Dicer will demonstrate a quicker and/or more robust response to dsRNA-mediated RNAi, and that cells that overexpress Dicer will respond even more quickly and/or more robustly. Overexpression of Dicer may be achieved by cloning a gene for a Dicer protein (e.g., the Drosophila DCR-1 gene), or orthologs or homologs thereof, into an expression vector or cassette that is placed into a cell of choice. Examples of cloned DCR genes include without limitation homologs and orthologs of DCR from mice (Nicholson, R. H. and Nicholson, A. W., Mamm. Genome 13:67-73 (2002)), accession No. NM_{—}148948; humans (Nagase, T., et al., DNA Res. 6:63-70 (1999)), accession No. NM_{—}030621; as well as the Drosophila Dicer-2 (DCR-2) gene (Adams, et al, Science 287:2185-2195 (2000)), accession No. NM_{—}079054.

References for siRNA

Adams et al. (2000). The genome sequence of Drosophila melanogaster. Science 287:2185-2195.

Bernstein, E., A. A. Caudy, S. M. Hammond and G. J. Hannon. (2001). Role for a bidentate ribonuclease in the initiation step of RNA interference. Nature 409:363-366.

Boutla, A., C. Delidakis, I. Livadaras, M. Tsagris and M. Tabler. (2001). Short 5′-phosphorylated double-stranded RNAs induce RNA interference in Drosophila. Curr Biol 11: 1776-1780.

Cullen B R. (2002). RNA interference: antiviral defense and genetic tool. Nat Immunol. 3:597-599.

Caplen, N. J., S. Parrish, F. Imani, A. Fire and R. A. Morgan. (2001). Specific inhibition of gene expression by small double-stranded RNAs in invertebrate and vertebrate systems. Proc Natl Acad Sci U S A 98:9742-9747.

Hamilton, A. J. and D. C. Baulcombe. (1999). A species of small antisense RNA in posttranscriptional gene silencing in plants. Science 286:950-952.

Nagase,T., Ishikawa,K., Suyama,M., Kikuno,R., Hirosawa,M., Miyajima,N., Tanaka,A., Kotani,H., Nomura,N. and Ohara,O. (1999). Prediction of the coding sequences of unidentified human genes. XIII. The complete sequences of 100 new cDNA clones from brain which code for large proteins in vitro. DNA Res. 6:63-70.

Napoli, C., C. Lemieux and R. Jorgensen. (1990). Introduction of a Chimeric Chalcone Synthase Gene into Petunia Results in Reversible Co-Suppression of Homologous Genes in trans. Plant Cell 2:279-289.

Nicholson, R. H. and Nicholson,A. W. (2002). Molecular characterization of a mouse cDNA encoding Dicer, a ribonuclease III ortholog involved in RNA interference. Mamm. Genome 13:67-73 (2002).

Parrish, S., J. Fleenor, S. Xu, C. Mello and A. Fire. (2000). Functional anatomy of a dsRNA trigger: differential requirement for the two trigger strands in RNA interference. Mol Cell 6:1077-1087.

Romano, N. and G. Macino. (1992). Quelling: transient inactivation of gene expression in Neurospora crassa by transformation with homologous sequences. Mol Microbiol 6:3343-3353.

Tabara, H., M. Sarkissian, W. G. Kelly, J. Fleenor, A. Grishok, L. Timmons, A. Fire and C. C. Mello. (1999). The rde-1 gene, RNA interference, and transposon silencing in C. elegans. Cell 99:123-132.

Tuschl T. RNA interference and small interfering RNAs. (2001). Chembiochem. 2:239-245.

b. Short Hairpin RNAs (shRNAs)

Paddison, P. J., et al., Genes & Dev. 16:948-958 (2002) have used small RNA molecules folded into hairpins as a means to effect RNAi. Accordingly, such short hairpin RNA (shRNA) molecules are also advantageously used in the compositions, complexes and methods of the present invention. The length of the stem and loop of functional shRNAs varies; stem lengths can range anywhere from about 25 to about 30 nt, and loop size can range between 4 to about 25 nt without affecting silencing activity. While not wishing to be bound by any particular theory, it is believed that these shRNAs resemble the dsRNA products of the Dicer RNase and, in any even, have the same capacity for inhibiting expression of a specific gene.

In order to express siRNA and shRNA long-term in vivo for, by way of non-limiting example, gene therapy and developmental studies, plasmids that express these RNAs have been generated. Expression vectors that continually express siRNAs in stably transfected mammalian cells have been developed. Other plasmids have been engineered to express small hairpin RNAs (shRNAs) lacking poly (A) tails. Transcription of shRNAs is initiated at a polymerase III (pol II) promoter and is believed to be terminated at position 2 of a 4-5-thymine transcription termination site. Upon expression, shRNAs are thought to fold into a stem-loop structure with 3′ UU-overhangs. Subsequently, the ends of these shRNAs are processed, converting the shRNAs into ˜21 nt siRNA-like molecules. The siRNA-like molecules can, in turn, bring about gene-specific silencing in the transfected cells, which may be, by way of non-limiting example, mammalian or human cells.

References for shRNA

Brummelkamp, T R, Bemards, R, and Agami, R. (2002). A system for stable expression of short interfering RNAs in mammalian cells. Science 296: 550-553.

Lee, N S, Dohjima, T, Bauer, G, Li, H, Li, M-J, Ehsani, A, Salvaterra, P, and Rossi, J. (2002). Expression of small interfering RNAs targeted against HIV-1 rev transcripts in human cells. Nature Biotechnol. 20:500-505.

Miyagishi, M, and Taira, K. (2002). U6-promoter-driven siRNAs with four uridine 3′ overhangs efficiently suppress targeted gene expression in mammalian cells. Nature Biotechnol. 20:497-500.

Paddison, P J, Caudy, A A, Bernstein, E, Hannon, G J, and Conklin, D S. (2002). Short hairpin RNAs (shRNAs) induce sequence-specific silencing in mammalian cells. Genes & Dev. 16:948-958.

Paul, C P, Good, P D, Winer, I, and Engelke, D R. (2002). Effective expression of small interfering RNA in human cells. Nature Biotechnol. 20:505-508.

Sui, G, Soohoo, C, Affar, E-B, Gay, F, Shi, Y, Forrester, W C, and Shi, Y. (2002). A DNA vector-based RNAi technology to suppress gene expression in mammalian cells. Proc. Natl. Acad. Sci. USA 99(6):5515-5520.

Yu, J-Y, DeRuiter, S L, and Turner, D L. (2002). RNA interference by expression of short-interfering RNAs and hairpin RNAs in mammalian cells. Proc. Natl. Acad. Sci. USA 99(9):6047-6052.

Elbashir, S M, Harborth, J, Lendeckel, W, Yalcin, A, Weber, K, and Tuschl, T. (2001) Duplexes of 21-nucleotide RNAs mediate RNA interference in mammalian cell culture. Nature 411:494-498.

c. Small Temporally Regulated RNAs (stRNAs)

Another group of small RNAs suitable for use in the compositions, complexes and methods of the present invention are the small temporally regulated RNAs (stRNAs). In general, stRNAs comprise from about 20 to about 30 nt (Banerjee and Slack, Control of development timing by small temporal RNAs: A paradigm for RNA-mediated regulation of gene expression, Bioessays 24:119-129, 2002). Unlike siRNAs, stRNAs downregulate expression of a target mRNA after the initiation of translation without degrading the mRNA.

d. Design and Synthesis of siRNA, shRNA, stRNA, Antisense and Other Oligonucleotides

One or more of the following guidelines may be used in designing the sequence of siRNA and other nucleic acids designed to bind to a target mRNA, e.g., shRNA, stRNA, antisense oligonucleotides, ribozymes, and the like, that are advantageously used in accordance with the present invention.

In the sequence of the target mRNA, select a region located from about 50 to about 100 nt 3′ from the start codon. In this region, search for the following sequences: AA(N₁₉)TT (SEQ ID NO:1) or AA(N₂₁) (SEQ ID NO:2), where N=any nucleotide. The GC content of the selected sequence should be from about 30% to about 70%, preferably about 50%. In order to maximize the specificity of the RNAi, it may be desirable to use the selected sequence in a search for related sequences in the genome of interest; sequences absent from other genes are preferred. The secondary structure of the target mRNA may be determined or predicted, and it may be preferable to select a region of the mRNA that has little or no secondary structure, but it should be noted that secondary structure seems to have little impact on RNAi. When possible, sequences that bind transcription and/or translation factors should be avoided, as they might competitively inhibit the binding of an siRNA, shRNA or stRNA (as well as other antisense oligonucleotides) to the mRNA. Thus, in general, it is preferred to select regions that do not overlap the start codon, and to also avoid the 5′ and 3′ untranslated regions (UTRs) of an mnRNA transcript.

Nucleic acids that mediate RNAi may be synthesized in vitro using methods to produce oligonucleotides and other nucleic acids, as is described elsewhere herein. In addition, dsRNA and other molecules that mediate iRNA are available from commercial vendors, such as Ribopharma AG (Kulmach, Germany), Eurogentec (Seraing, Belgium) and Sequitur (Natick, Mass.). Eurogentec offers siRNA that has been labeled with fluorophores (e.g., HEX/TET; 5′ Fluorescein, 6-FAM; 3′ Fluorescein, 6-FAM; Fluorescein dT internal; 5′ TAMRA, Rhodamine; 3′ TAMRA, Rhodamine), and these examples of fluorescent dsRNA that can be used in the invention.

4. Aptamers

Traditionally, techniques for detecting and purifying target molecules have used polypeptides, such as antibodies, that specifically bind such targets. Nucleic acids have long been known to specifically bind other nucleic acids (e.g., ones having complementary sequences).

However, nucleic acids that bind non-nucleic target molecules have been described and are generally referred to as aptamers. See, e.g., Blackwell, T. K., et al., Science (1990) 250:1104-1110; Blackwell, T. K., et al., Science (1990) 250:1149-1152; Tuerk, C., and Gold, L., Science (1990) 249:505-510; Joyce, G. F., Gene (1989) 82:83-87. Accordingly, nucleic acid molecules (e.g., oligonucleotides) suitable for use in the present invention also include aptamers.

As applied to aptamers, the term “binding” specifically excludes the “Watson-Crick”-type binding interactions (i.e., A:T and G:C base-pairing) traditionally associated with the DNA double helix.

The term “aptamer” thus refers to a nucleic acid or a nucleic acid derivative that specifically binds to a target molecule, wherein the target molecule is either (i) not a nucleic acid, or (ii) a nucleic acid or structural element thereof that is bound by the aptatmer through mechanisms other than duplex- or triplex-type base pairing.

In general, techniques for identifying aptamers involve incubating a preselected non-nucleic acid target molecule with mixtures (2 to 50 members), pools (50 to 5,000 members) or libraries (50 or more members) of different nucleic acids that are potential aptamers under conditions that allow complexes of target molecules and aptamers to form. By “different nucleic acids” it is meant that the nucleotide sequence of each potential aptamer may be different from that of any other member, that is, the sequences of the potential aptamers are random with respect to each other. Randomness can be introduced in a variety of manners such as, e.g., mutagenesis, which can be carried out in vivo by exposing cells harboring a nucleic acid with mutagenic agents, in vitro by chemical treatment of a nucleic acid, or in vitro by biochemical replication (e.g., PCR) that is deliberately allowed to proceed under conditions that reduce fidelity of replication process; randomized chemical synthesis, i.e., by synthesizing a plurality of nucleic acids having a preselected sequence that, with regards to at least one position in the sequence, is random. By “random at a position in a preselected sequence” it is meant that a position in a sequence that is normally synthesized as, e.g., as close to 100% A as possible (e.g., 5′-C-T-T-A-G-T-3′), is allowed to be randomly synthesized at that position (C-T-T-N-G-T, wherein N indicates a randomized position. At a randomized position, for example, the synthesizing reaction contains 25% each of A,T,C and G; or x % A, w % T, y % C and z % G, wherein x+w+y+z=100. The randomization at the position may be complete (i.e., x=y=w=z=25%) or stoichastic (i.e., at least one of x, w, y and z is not 25%).

In later stages of the process, the sequences are increasingly less randomized and consensus sequences may appear; in any event, it is preferred to ultimately obtain an aptamer having a unique nucleotide sequence.

Aptamers and pools of aptamers are prepared, identified, characterized and/or purified by any appropriate technique, including those utilizing in vitro synthesis, recombinant DNA techniques, PCR amplification, and the like. After their formation, target:aptamer complexes are then separated from the uncomplexed members of the nucleic acid mixture, and the nucleic acids that can be prepared from the complexes are candidate aptamers (at early stages of the technique, the aptamers generally being a population of a multiplicity of nucleotide sequences having varying degrees of specificity for the target). The resulting aptamer (mixture or pool) is then substituted for the starting apatamer (library or pool) in repeated iterations of this series of steps. When a limited number (e.g., a pool or mixture, preferably a mixture with less than 10 members, most preferably 1) of nucleic acids having satisfactory specificity is obtained, the aptamer is sequenced and characterized. Pure preparations of a given aptamer are generated by any appropriate technique (e.g., PCR amplification, in vitro chemical synthesis, and the like).

For example, Tuerk and Gold (Science (1990) 249:505-510) describe the use of a procedure termed “systematic evolution of ligands by exponential enrichment” (SELEX). In this method, pools of nucleic acid molecules that are randomized at specific positions are subjected to selection for binding to a nucleic acid-binding protein (see, e.g., PCT International Publication No. WO 91/19813 and U.S. Pat. No. 5,270,163). The oligonucleotides so obtained are sequenced and otherwise characterization. Kinzler, K. W., et al. (Nucleic Acids Res. (1989) 17:3645-3653) used a similar technique to identify synthetic double-stranded DNA molecules that are specifically bound by DNA-binding polypeptides. Ellington, A. D., et al. (Nature (1990) 346: 818-822) describe the production of a large number of random sequence RNA molecules and the selection and identification of those that bind specifically to specific dyes such as Cibacron blue.

Another technique for identifying nucleic acids that bind non-nucleic target molecules is the oligonucleotide combinatorial technique described by Ecker, D. J. et al. (Nuc. Acids Res. 21, 1853 (1993)) known as “synthetic unrandomization of randomized fragments” (SURF), which is based on repetitive synthesis and screening of increasingly simplified sets of oligonucleotide analogue libraries, pools and mixtures (Tuerk, C. and Gold, L. (Science 249, 505 (1990)). The starting library consists of oligonucleotide analogues of defined length with one position in each pool containing a known analogue and the remaining positions containing equimolar mixtures of all other analogues. With each round of synthesis and selection, the identity of at least one position of the oligomer is determined until the sequences of optimized nucleic acid ligand aptamers are discovered.

Once a particular candidate aptamer has been identified through a SURF, SELEX or any other technique, its nucleotide sequence can be determined (as is known in the art), and its three-dimensional molecular structure can be examined by nuclear magnetic resonance (NMR). These techniques are explained in relation to the determination of the three-dimensional structure of a nucleic acid ligand that binds thrombin in Padmanabhan, K. et al., J. Biol. Chem. 24, 17651 (1993); Wang, K. Y. et al., Biochemistry 32, 1899 (1993); and Macaya, R. F. et al., Proc. Nat'l. Acad. Sci. USA 90, 3745 (1993). Selected aptamers may be resynthesized using one or more modified bases, sugars or backbone linkages. Aptamers consist essentially of the minimum sequence of nucleic acid needed to confer binding specificity, but may be extended on the 5′ end, the 3′ end, or both, or may be otherwise derivatized or conjugated.

5. Oligonucleotide Synthesis

The oligonucleotides used in accordance with the present invention can be conveniently and routinely made through the well-known technique of solid-phase synthesis. Equipment for such synthesis is sold by several vendors including, for example, Applied Biosystems (Foster City, Calif.). Other methods for such synthesis that are known in the art may additionally or alternatively be employed. It is well known to use similar techniques to prepare oligonucleotides such as the phosphorothioates and alkylated derivatives. By way of non-limiting example, see, e.g., U.S. Pat. No. 4,517,338 (Multiple reactor system and method for polynucleotide synthesis) to Urdea et al., and U.S. Pat. No. 4,458,066 (Process for preparing polynucleotides) to Caruthers et al.; Lyer R P, Roland A, Zhou W, Ghosh K. Modified oligonucleotides—synthesis, properties and applications. Curr Opin Mol Ther. 1:344-358, 1999; Verma S, Eckstein F. Modified oligonucleotides: synthesis and strategy for users. Annu Rev Biochem. 67:99-134, 1998; Pfleiderer W, Matysiak S, Bergmann F, Schnell R. Recent progress in oligonucleotide synthesis. Acta Biochim Pol. 43:37-44, 1996; Warren W J, Vella G. Principles and methods for the analysis and purification of synthetic deoxyribonucleotides by high-performance liquid chromatography. Mol Biotechnol. 4:179-199, 1995; Sproat B S. Chemistry and applications of oligonucleotide analogues. J Biotechnol. 41:221-238, 1995; De Mesmaeker A, Altmann K H, Waldner A, Wendeborn S. Backbone modifications in oligonucleotides and peptide nucleic acid systems. Curr Opin Struct Biol. 5:343-355, 1995; Charubala R, Pfleiderer W. Chemical synthesis of 2′,5′-oligoadenylate analogues. Prog Mol Subcell Biol. 14:114-138, 1994; Sonveaux E. Protecting groups in oligonucleotide synthesis. Methods Mol Biol. 26:1-71, 1994; Goodchild J. Conjugates of oligonucleotides and modified oligonucleotides: a review of their synthesis and properties. Bioconjug Chem. 1:165-187, 1990; Thuong N T, Asseline U. Chemical synthesis of natural and modified oligodeoxynucleotides. Biochimie. 67:673-684, 1985; Itakura K, Rossi J J, Wallace R B. Synthesis and use of synthetic oligonucleotides. Annu Rev Biochem. 53:323-356, 1984; Caruthers M H, Beaucage S L, Becker C, Efcavitch J W, Fisher E F, Galluppi G, Goldman R, deHaseth P, Matteucci M, McBride L, et al. Deoxyoligonucleotide synthesis via the phosphoramidite method. Gene Amplif Anal. 3:1-26, 1983; Ohtsuka E, Ikehara M, Soll D. Recent developments in the chemical synthesis of polynucleotides. Nucleic Acids Res. 10:6553-6560, 1982; and Kossel H. Recent advances in polynucleotide synthesis. Fortschr Chem Org Naturst. 32:297-508, 1975.

Oligonucleotides and other nucleic acids having accessory elements can also be prepared for advantageous use in the compositions, complexes and methods of the present invention. Some such accessory elements can specifically bind or otherwise interact with another molecule for a variety of purposes, including without limitation:

Intracellular transport. For example, a nucleotide sequence that localizes nucleic acids to mitochondria is described in U.S. Pat. No. 5,569,754;

Cellular targeting. For example, the sequence of an aptamer that binds to a cell surface molecule (e.g., a receptor, cellular adhesion protein, membrane lipid, etc.) can be included in order to direct the oligonucleotide or other nucleic acid to a particular type of cell;

Delivery of DNA-binding proteins. For example, a nucleotide sequence that specifically binds a transcription factor can be included in order to effect the delivery of the transcription factor at the same time as the other components of the complex;

Delivery of recombination proteins. As an example, a site that specifically binds a recombination protein can be included. The recombination protein can be a recombinase per se (e.g., lambda integrase and related site-specific recombinases) or a protein that facilitates or enhances recombination (e.g., a histonelike protein, such as Integration Host Factor, IHF). In one embodiment, a histonelike protein (e.g., IHF) and a site-specific recombinase (e.g., lambda integrase or Xis) are incorporated into one or more complexes, and cells are transfected therewith. The presence of IHF in transfected cells increases the amount of site-specific recombination mediated by the integrase, thereby promoting recombination between specific sites (e.g., attB, attP, attL, attR, etc.) on nucleic acids within the cells (Christ et al., 2002. Site-specific recombination in eukaryotic cells mediated by mutant lambda integrases: implications for synaptic complex formation and the reactivity of episomal DNA segments. J Mol Biol 319:305-314). Such cells include, without limitation, embryonic cells, such as stem cells (Christ N, Droge P. 2002. Genetic manipulation of mouse embryonic stem cells by mutant lambda integrase. Genesis 32:203-208). In another embodiment, mutants of lambda integrase that have activity in the absence of IHF are used (Lorbach et al., 2000. Site-specific recombination in human cells catalyzed by phage lambda integrase mutants. J Mol Biol 296:1175-81).

6. Chemical Modifications of Nucleic Acids

In certain embodiments, oligonucleotides used in accordance with the present invention may comprise one or more chemical modifications including with neither limitation nor exclusivity base modifications, sugar modifications, and backbone modifications. In addition, a variety of molecules can be conjugated to the oligonucleotides; see, e.g., the descriptions of chemical conjunction of fluorophores to oligonucleotides that are present throughout the present disclosure. Other suitable modifications include but are not limited to base modifications, sugar modifications, backbone modifications, and the like.

a. Base Modifications

In certain embodiments, the oligonucleotides used in the present invention can comprise one or more base modifications. For example, the base residues in aptamers may be other than naturally occurring bases (e.g., A, G, C, T, U, and the like). Derivatives of purines and pyrimidines are known in the art; an exemplary but not exhaustive list includes aziridinylcytosine, 4-acetylcytosine, 5-fluorouracil, 5-bromouracil, 5-carboxymethylaminomethyl-2-thiouracil, 5-carboxymethylaminomethyluracil, inosine (and derivatives thereof), N6-isopentenyladenine, 1-methyladenine, 1-methylpseudouracil, 1-methylguanine, 1-methylinosine, 2,2-dimethylguanine, 2-methyladenine, 2-methylguanine, 7-methylguanine, 3-methylcytosine, 5-methylcytosine (5MC), N6-methyladenine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. In addition to nucleic acids that incorporate one or more of such base derivatives, nucleic acids having nucleotide residues that are devoid of a purine or a pyrimidine base may also be included in oligonucleotides and other nucleic acids.

b. Sugar Modifications

The oligonucleotides used in the present invention can also (or alternatively) comprise one or more sugar modifications. For example, the sugar residues in oligonucleotides and other nucleic acids may be other than conventional ribose and deoxyribose residues. By way of non-limiting example, substitution at the 2′-position of the furanose residue enhances nuclease stability. An exemplary, but not exhaustive list, of modified sugar residues includes 2′ substituted sugars such as 2′-O-methyl-, 2′-O-alkyl, 2′-O-allyl, 2′-S-alkyl, 2′-S-allyl, 2′-fluoro-, 2′-halo, or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside, ethyl riboside or propylriboside.

c. Backbone Modifications

The oligonucleotides used in the present invention can also (or alternatively) comprise one or more backbone modifications. For example, chemically modified backbones of oligonucleotides and other nucleic acids include, by way of non-limiting example, phosphorothioates, chiral phosphorothioates, phosphorodithioates, phosphotriesters, aminoalkylphos-photriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotri-esters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Chemically modified backbones that do not contain a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages, including without limitation morpholino linkages; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; and amide backbones.

B. Vectors and Constructs

In certain embodiments, the nucleic acid molecules of the invention are provided as vectors, particularly cloning vectors, expression vectors or gene therapy vectors. Vectors according to this aspect of the invention can be double-stranded or single-stranded and which may be DNA, RNA, or DNA/RNA hybrid molecules, in any conformation including but not limited to linear, circular, coiled, supercoiled, torsional, nicked and the like. These vectors of the invention include but are not limited to plasmid vectors and viral vectors, such as a bacteriophage, baculovirus, retrovirus, lentivirus, adenovirus, vaccinia virus, semliki forest virus and adeno-associated virus vectors, all of which are well-known and can be purchased from commercial sources (Invitrogen; Carlsbad, Calif.; Promega, Madison Wis.; Stratagene, La Jolla Calif.).

In accordance with the invention, any vector may be used to construct the cloning vectors and expression vectors of the invention. In particular, vectors known in the art and those commercially available (and variants or derivatives thereof) may in accordance with the invention be engineered to include one or more recombination sites for use in the methods of the invention. Such vectors may be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, Perkin Elmer, Pharmingen, Research Genetics. General classes of vectors of particular interest include prokaryotic and/or eukaryotic cloning vectors, expression vectors, fusion vectors, two-hybrid or reverse two-hybrid vectors, shuttle vectors for use in different hosts, mutagenesis vectors, transcription vectors, vectors for receiving large inserts and the like. Other vectors of interest include viral origin vectors (M13 vectors, bacterial phage λ vectors, adenovirus vectors, and retrovirus vectors), high, low and adjustable copy number vectors, vectors which have compatible replicons for use in combination in a single host (pACYC184 and pBR322) and eukaryotic episomal replication vectors (pCDM8).

Particular vectors of interest include prokaryotic expression vectors such as pProEx-HT, pcDNA II, pSL301, pSE280, pSE380, pSE420, pTrcHisA, B, and C, pRSET A, B, and C (Invitrogen Corporation), pGEMEX-1, and pGEMEX-2 (Promega, Inc.), the pET vectors (Novagen, Inc.), pTrc99A, pKK223-3, the pGEX vectors, pEZZ18, pRIT2T, and pMC1871 (Pharmacia, Inc.), pKK233-2 and pKK388-1 (Clontech, Inc.), and variants and derivatives thereof. Vectors can also be made from eukaryotic expression vectors such as pYES2, pAC360, pBlueBacHis A, B, and C, pVL1392, pBsueBacIII, pCDM8, pcDNA1, pZeoSV, pcDNA3 pREP4, pCEP4, pEBVHis, pFastBac, pFastBac HT, pFastBac DUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMC1neo, and pOG44 (Stratagene, Inc.), and variants or derivatives thereof.

Other vectors of particular interest include pUC18, pUC19, pBlueScript, pSPORT, cosmids, phagemids, YACs (yeast artificial chromosomes), BACs (bacterial artificial chromosomes), MACs (mammalian artificial chromosomes), HACs (human artificial chromosomes), P1 (E. coli phage), pQE70, pQE60, pQE9 (Qiagen), pBS vectors, PhageScript vectors, BlueScript vectors, pNH8A, pNH16A, pNH18A, pNH46A (Stratagene), pcDNA3, pSPORT1, pSPORT2, pCMVSPORT2.0 and pSV-SPORT1 (Invitrogen), pGEX, pTrsfus, pTrc99A, pET-5, pET-9, pKK223-3, pKK233-3, pDR540, pRIT5 (Pharmacia), and variants or derivatives thereof.

Additional vectors of interest include pTrxFus, pThioHis, pLEX, pTrcHis, pTrcHis2, pRSET, pBlueBacHis2, pcDNA3.1/His, pcDNA3.1(−)/Myc-His, pSecTag, pEBVHis, pPIC9K, pPIC3.5K, pAO815, pPICZ, pPICZa, pGAPZ, pGAPZa, pBlueBac4.5, pBlueBacHis2, pMelBac, pSinRep5, pSinHis, pIND, pIND(SP1), pVgRXR, pcDNA2.1. pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe,SV2, pRc/CMV2, pRc/RSV, pREP4, pREP7, pREP8, pREP9, pREP10, pCEP4, pEBVHis, pCR3.1, pCR2.1, pCR3.1-Uni, and pCRBac from Invitrogen; λExCell, λgt11, pTrc99A, pKK223-3, pGEX-1λT, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-1b(+), pT7Blue(R), pT7Blue-2, pCITE-4abc(+), pOCUS-2, pTAg, pET-32 LIC, pET-30 LIC, pBAC-2cp LIC, pBACgus-2cp LIC, pT7Blue-2 LIC, pT7Blue-2, ASCREEN-1, λBlueSTAR, pET-3abcd, pET-7abc, pET9abcd, pET11abcd, pET12abc, pET-14b, pET-15b, pET-16b, pET-17b- pET-17xb, pET-19b, pET-20b(+), pET-21abcd(+), pET-22b(+), pET-23abcd(+), pET-24abcd(+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3cp, pBACgus-2cp, pBACsurf-1, plg, Signal plg, pYX, Selecta Vecta-Neo, Selecta Vecta—Hyg, and Selecta Vecta—Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2-Basic, pSEAP2-Contral, pSEAP2-Promoter, pSEAP2-Enhancer, pβgal-Basic, pβgal-Control, pβgal-Promoter, pβgal-Enhancer, pCMVβ, pTet-Off, pTet-On, pTK-Hyg, pRetro-Off, pRetro-On, pIRES1neo, pIRES1hyg, pLXSN, pLNCX, pLAPSN, pMAMneo, pMAMneo-CAT, pMAMneo-LUC, pPUR, pSV2neo, pYEX 4T-1/2/3, pYEX-S1, pBacPAK-His, pBacPAK8/9, pAcUW31, BacPAK6, pTriplEx, λgt10, λgt11, pWE15, and λTriplEx from Clontech; Lambda ZAP II, pBK-CMV, pBK-RSV, pBluescript II KS ±, pBluescript II SK ±, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS ±, pBC KS ±, pBC SK ±, Phagescript, pCAL-n-EK, pCAL-n, pCAL-c, pCAL-kc, pET-3abcd, pET-11abcd, pSPUTK, pESP-1, pCMVLacI, pOPRSVI/MCS, pOPI3 CAT, pXT1, pSG5, pPbac, pMbac, pMC1neo, pMC1neo Poly A, pOG44, pOG45, PFRTβGAL, pNEOβGAL, pRS403, pRS404, pRS405, pRS406, pRS413, pRS414, pRS415, and pRS416 from Stratagene.

Two-hybrid and reverse two-hybrid vectors of particular interest include pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp and variants or derivatives thereof.

Other suitable vectors will be readily apparent to the skilled artisan.

1. Cloning Vectors

Cloning vectors according to the invention include plasmids, cosmids, viral or phage DNA molecules or other DNA molecules that are capable of autonomous replication in a host cell, via splicing of vector-borne nucleic acid into the genetic material (chromosomal or extrachromosomal) of the host cell without loss of an essential biological function of the vector, thereby facilitating the replication and cloning of the vector. The cloning vector may further contain a marker suitable for use in the identification of cells transformed with the cloning vector. Markers may be, for example, antibiotic resistance genes, e.g., tetracycline resistance or ampicillin resistance. Clearly, methods of inserting a desired nucleic acid fragment which do not require the use of homologous recombination, transpositions or restriction enzymes (such as, but not limited to, UDG cloning of PCR fragments (U.S. Pat. No. 5,334,575, entirely incorporated herein by reference), T:A cloning, and the like) can also be applied to clone a fragment into a cloning vector to be used according to the present invention. The cloning vector can further contain one or more selectable markers suitable for use in the identification of cells transformed with the cloning vector.

2. Expression Vectors

Expression vectors according to the invention include vectors that are capable of enhancing the expression of one or more genes that have been inserted or cloned into the vector, upon transformation of the vector into a host. The cloned gene is usually placed under the control of (i.e., operably linked to) certain transcriptional regulatory sequences such as promoter sequences. In certain preferred embodiments in this regard, the vectors provide for specific expression, which may be inducible and/or cell type-specific. Particularly preferred among such vectors are those inducible by environmental factors that are easy to manipulate, such as temperature and nutrient additives. Expression vectors useful in the present invention include chromosomal-, episomal- and virus-derived vectors, e.g., vectors derived from bacterial plasmids or bacteriophages, and vectors derived from combinations thereof, such as cosmids and phagemids.

To produce expression vectors according to this aspect of the invention, one or more gene-containing nucleic acid molecules or oligonucleotide inserts should be operatively linked to an appropriate promoter in the vector (which may be provided by the vector itself (i.e., a “homologous promoter”) or may be exogenous to the vector (i.e., a “heterologous promoter), such as the phage lambda P_{L} promoter, the E. coli lac, trp and tac promoters, and the like. Other suitable promoters will be known to the skilled artisan. The gene fusion constructs will further contain sites for transcription initiation, termination and, in the transcribed region, a ribosome binding site for translation. The coding portion of the mature transcripts expressed by the constructs will preferably include a translation initiation codon at the beginning, and a termination codon (UAA, UGA or UAG) appropriately positioned at the end, of the polynucleotide to be translated. The expression vectors also preferably include at least one selectable marker. Such markers include tetracycline or ampicillin resistance genes for culturing in E. coli and other bacteria.

Viral expression vectors can be particularly useful where a method of the invention is practiced for the purpose of generating a ds recombinant nucleic acid molecule covalently linked in one or both strands, that is to be introduced into a cell, particularly a cell in a subject. Viral vectors provide the advantage that they can infect host cells with relatively high efficiency and can infect specific cell types or can be modified to infect particular cells in a host.

Viral vectors have been developed for use in particular host systems and include, for example, bacteriophage vectors (e.g., phage lambda), which infect bacterial cells (for review, see Baneyx F., Curr Opin. Biotechnol. 10:411-421 (1999)), baculovirus vectors, which infect insect cells; retroviral vectors, other lentivirus vectors such as those based on the human immunodeficiency virus (HIV), adenovirus vectors, adeno-associated virus (AAV) vectors, herpesvirus vectors, vaccinia virus vectors, and the like, which infect mammalian cells (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187 (1996), each of which is incorporated herein by reference). For example, a viral vector based on an HIV can be used to infect T cells, a viral vector based on an adenovirus can be used, for example, to infect respiratory epithelial cells, and a viral vector based on a herpesvirus can be used to infect neuronal cells. Other vectors, such as AAV vectors can have greater host cell range and, therefore, can be used to infect various cell types, although viral or non-viral vectors also can be modified with specific receptors or ligands to alter target specificity through receptor mediated events.

3. Vectors, Compositions and Methods for Gene Therapy

In additional embodiments, the invention provides compositions comprising one or more genetic constructs, including vectors (such as the expression or cloning vectors described above), or one or more of the complexes of the invention, that may be useful in delivering nucleic acid molecules to cells, tissues, organs and organisms for therapeutic or prophylactic purposes. The invention further provides methods for preparing nucleic acid molecules having regions of viral nucleic acids, as well as nucleic acid molecules prepared by such methods and compositions comprising these nucleic acid molecules, useful for the nucleic acid delivery and therapeutic/prophylactic purposes described above and in more detail below.

In one embodiment, the present invention provides methods for treating or preventing a physical disorder in an animal that is suffering from or predisposed to the physical disorder, comprising introducing into the animal one or more of the nucleic acid molecules, complexes or compositions of the invention. According to the invention, an animal, particularly a mammal (preferably a human) that is suffering from, or that is predisposed or susceptible to, a physical disorder may be treated by administering to the animal an effective dose of one or more of the nucleic acid molecules, complexes or compositions of the invention, optionally in combination with a pharmaceutically acceptable carrier or excipient therefor. As used herein, an animal that is “suffering from” a particular physical disorder is defined as an animal that exhibits one or more overt physical symptoms of the disorder that are typically used in the diagnosis or identification of the disorder according to established medical and veterinary procedures and protocols that will be familiar to the ordinarily skilled artisan. Analogously, as used herein, an animal that is “predisposed to” or “susceptible to” a physical disorder is defined as an animal that does not exhibit a plurality of overt physical symptoms of the disorder but that is genetically, physiologically or otherwise at risk for developing the disorder under appropriate physiological and environmental conditions. Hence, whether or not a particular animal is “suffering from,” “predisposed to” or “susceptible to” a particular physical disorder will be apparent to the ordinarily skilled artisan upon determination of the medical history of the animal using methods that are routine in the medical and veterinary arts.

Physical disorders treatable or preventable with the compositions and methods of the present invention include any physical disorder that may be delayed, prevented, cured or otherwise treated by modulating immune system function, particularly activation and/or apoptosis in antigen-presenting cells, in an animal suffering from, or predisposed or susceptible to, the physical disorder. Such physical disorders that may be treatable or preventable using the compositions, complexes and methods of the present invention include, but are not limited to, infectious diseases (particularly bacterial diseases (including without limitation meningitis, pneumonia, tetanus, cholera, typhoid fever, staphylococcal skin infections, streptococcal pharyngitis, scarlet fever, pertussis, diphtheria, tuberculosis, leprosy, rickettsial diseases, bacteremia, bacterial venereal diseases and the like), viral diseases (including without limitation meningitis, AIDS, influenza, rhinitis, hepatitis, polio, pneumonia, yellow fever, Lassa fever, Ebola fever and the like), and/or fungal diseases (including without limitation cryptococcosis, blastomycosis, mucormycosis, histoplasmosis, aspergillosis, and the like), parasitic diseases (including without limitation malaria, Leishmaniasis, filariasis, trypanasomiasis, schistosomiasis, and the like), cancers (such as carcinomas, melanomas,, sarcomas, leukemias and the like), and other disorders treatable or preventable using the methods and compositions of the present invention. Analogously, physical disorders that may be treatable or preventable using the present compositions and methods include, but are not limited to, immune system disorders (such as rheumatoid arthritis, multiple sclerosis, systemic lupus erythematosis, Crohn's Disease), and other disorders of analogous etiology. The compositions and methods of the present invention may also be used in the prevention of disease progression, such as in chemoprevention of the progression of a premalignant lesion to a malignant lesion, and to treat an animal suffering from, or predisposed to, other physical disorders that respond to treatment with compositions that activate, or inhibit/delay/prevent or induce apoptosis in, antigen-presenting cells.

In a first such aspect of the invention, the animal suffering from or predisposed to a physical disorder may be treated by introducing into the animal one or more of the nucleic acid molecules of the invention, optionally in the form of a vector and further optionally in the form of a polypeptide-nucleic acid complex of the invention (or a composition of the invention comprising one or more such complexes). This approach, known generically as “gene therapy,” is designed to increase the level of expression of a given gene, generally contained on the nucleic acid molecule and/or in the administered complex, in the cells and/or tissues of the animal, thereby inhibiting, delaying or preventing the progression and/or development of the physical disorder, or to induce the reversal, amelioration or remission of one or more overt symptoms or processes of the physical disorder. Analogous gene therapy approaches have proven effective or to have promise in the treatment of a variety of mammalian diseases such as cystic fibrosis (Drumm, M. L. et al., Cell 62:1227-1233 (1990); Gregory, R. J. et al., Nature 347:358-363 (1990); Rich, D. P. et al., Nature 347:358-363 (1990)), Gaucher disease (Sorge, J. et al., Proc. Natl. Acad. Sci. USA 84:906-909 (1987); Fink, J. K. et al., Proc. Natl. Acad. Sci. USA 87:2334-2338 (1990)), certain forms of hemophilia (Bontempo, F. A. et al., Blood 69:1721-1724 (1987); Palmer, T. D. et al., Blood 73:438-445 (1989); Axelrod, J. H. et al., Proc. Natl. Acad. Sci. USA 87:5173-5177 (1990); Armentano, D. et al., Proc. Natl. Acad. Sci. USA 87:6141-6145 (1990)) and muscular dystrophy (Partridge, T. A. et al., Nature 337:176-179 (1989); Law, P. K. et al., Lancet 336:114-115 (1990); Morgan, J. E. et al., J. Cell Biol. 111:2437-2449 (1990)), and certain cancers such as metastatic melanoma (Rosenberg, S. A. et al., Science 233:1318-1321 (1986); Rosenberg, S. A. et al., N. Eng. J. Med. 319:1676-1680 (1988); Rosenberg, S. A. et al., N. Eng. J. Med. 323:570-578 (1990)).

In carrying out such gene therapy methods of the invention, a variety of vectors, particularly viral vectors, are useful in forming the complexes and compositions of the invention. For example, adenoviruses are especially attractive vehicles for delivering genes to or via respiratory epithelia and the use of such vectors are included within the scope of the invention. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993), present a review of adenovirus-based gene therapy. Bout et al., Human Gene Therapy 5:3-10 (1994), demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication Nos. WO94/12649 and WO 96/17053; U.S. Pat. No. 5,998,205; and Wang et al., Gene Therapy 2:775-783 (1995), the disclosures of all of which are incorporated herein by reference in their entireties. Adeno-associated viruses (AAV) and Herpes viruses, as well as vectors prepared from these viruses have also been proposed for use in gene therapy (Walsh et al., 1993, Proc. Soc. Exp. Biol. Med. 204:289-300; U.S. Pat. No. 5,436,146; Wagstaff et al., Gene Ther. 5:1566-70 (1998)). Herpes viral vectors are particularly useful for applications where gene expression is desired in nerve cells.

In a preferred such approach, one or more nucleic acid molecules of the invention, or one or more polypeptide-nucleic acid complexes of the invention, is introduced into or administered to the animal that is suffering from or predisposed to the physical disorder. Such nucleic acid molecules may be incorporated into a vector or virion suitable for introducing the nucleic acid molecules into the cells or tissues of the animal to be treated, to form a transfection vector. Suitable vectors or virions for this purpose include those derived from retroviruses, adenoviruses, alphaviruses, herpes viruses and adeno-associated viruses. Alternatively, the nucleic acid molecules of the invention may be complexed into a molecular conjugate with a virus (e.g., an adenovirus or an adeno-associated virus) or with viral components (e.g., viral capsid proteins), which optionally can be further complexed with one or more polypeptides into a polypeptide-nucleic acid complex of the invention. As one of ordinary skill will readily recognize, the nucleic acid molecules and/or complexes of the invention also optionally may be combined with one or more pharmaceutically acceptable excipients or diluents to form a pharmaceutical composition suitable for use in these methods of the invention.

Techniques for the formation of vectors or virions comprising nucleic acid molecules of the invention are well-known in the art, and are generally described in “Working Toward Human Gene Therapy,” Chapter 28 in Recombinant DNA, 2nd Ed., Watson, J. D. et al., eds., New York: Scientific American Books, pp. 567-581 (1992). In addition, general methods for construction of gene therapy vectors and the introduction thereof into affected animals for therapeutic purposes may be obtained in the above-referenced publications, the disclosures of which are specifically incorporated herein by reference in their entirety. In one such general method, vectors comprising the nucleic acid molecules of the present invention are directly introduced into the cells or tissues of the affected animal, preferably by injection, inhalation, ingestion or introduction into a mucous membrane via solution; such an approach is generally referred to as “in vivo” gene therapy. Alternatively, cells, tissues or organs, particularly those containing one or more defective or nonfunctioning genes, containing pathological agents (e.g., bacteria, viruses, parasites, yeasts, etc.), or containing cancer cells or tumors, may be removed from the affected animal and placed into culture according to methods that are well-known to one of ordinary skill in the art. The vectors comprising the nucleic acid molecules of the invention, typically comprising one or more therapeutic genes or nucleic acid sequences, may then be introduced into these cells or tissues by any of the methods described generally above for introducing oligonucleotides into a cell or tissue, and, after a sufficient amount of time to allow incorporation of the oligonucleotides, the cells or tissues may then be re-inserted into the affected animal. Since the introduction of the therapeutic genese or nucleic acid sequences is performed outside of the body of the affected animal, this approach is generally referred to as “ex vivo” gene therapy.

For both in vivo and ex vivo gene therapy, the nucleic acid molecules (e.g., oligonucleotides) of the invention may alternatively be operatively linked to a regulatory DNA sequence, which may be a promoter or an enhancer, or a heterologous regulatory DNA sequence such as a promoter or enhancer derived from a gene, cell or organism different from that used as the source of the nucleic acid molecule being used in gene therapy, to form a genetic construct as described above. This genetic construct may then be inserted into a vector, which is then directly introduced into the affected animal in an in vivo gene therapy approach, or into the cells or tissues of the affected animal in an ex vivo approach. In another embodiment, the genetic construct of the invention may be introduced into the cells or tissues of the animal, either in vivo or ex vivo, in a molecular conjugate with a virus (e.g., an adenovirus or an adeno-associated virus) or viral components (e.g., viral capsid proteins; see WO 93/07283). In yet another embodiment, the genetic construct of the invention may be introduced into the animal in the form of a polypeptide-nucleic acid complex of the invention. Alternatively, transfected host cells, which may be homologous or heterologous, may be encapsulated within a semi-permeable barrier device and implanted into the affected animal, allowing passage of one or more therapeutic polypeptides encoded by the nucleic acid molecules in the conjugate or complex of the invention into the tissues and circulation of the animal, but preventing contact between the animal's immune system and the transfected cells (see WO 93/09222). These approaches result in increased production of one or more therapeutic polypeptides by the treated animal via (a) random insertion of the therapeutic gene (contained on the nucleic acid molecule of the invention) into the host cell genome; or (b) incorporation of the therapeutic gene into the nucleus of the cells where it may exist as an extrachromosomal genetic element. General descriptions of such methods and approaches to gene therapy may be found, for example, in U.S. Pat. No. 5,578,461; WO 94/12650; and WO 93/09222; the disclosures of all of which are incorporated herein by reference in their entireties.

The invention thus includes methods for preparing nucleic acid molecules which have one or more functional properties of viral vectors (e.g., adenoviral vectors, alphaviral vectors, herpes viral vectors, adeno-associated viral vectors, etc.). In particular embodiments, methods of the invention include the joining of nucleic acid segments, wherein one or more of the nucleic acid segments contains regions which confer upon product nucleic acid molecules the ability to function as viral vectors (e.g., the ability to replicate in specific host cells, the ability to be packaged into viral particles, etc.).

In particular embodiments, the invention includes methods for preparing adenoviral vectors by joining at least one (e.g., one, two, three, four, etc.) nucleic acid segment which comprises adenoviral sequences to one or more other nucleic acid segments. Specific examples of adenoviral vectors, and nucleic acid segments which can be used to prepare adenoviral vectors are disclosed in U.S. Pat. Nos. 5,932,210, 6,136,594, and 6,303,362, the entire disclosures of which are incorporated herein by reference. Adenoviral vector prepared by methods of the invention may be replication competent or replication deficient.

One example of an adenoviral vector may be prepared by joining a nucleic acid segment comprising adenoviral nucleic acid to one or more other nucleic acid segments. For example, when a replication deficient adenoviral vector is desired, the adenoviral nucleic acid may have deletions of all or part of one or more of the following regions: the E1a region, the E1b region, and/or the E3 region. Adenoviral vectors which contain deletions in these regions are described, for example, in U.S. Pat. No. 6,136,594. The invention further includes adenoviral vectors prepared by methods of the invention, as well as uses of these vectors and compositions comprising these vectors. One example of a use of adenoviral vectors prepared by methods of the invention include the delivery of nucleic acid segments to cells of a mammal (e.g., a human). Thus, the invention provides methods for preparing vector suitable for use in gene therapy protocols. Typically, such vectors will be replication deficient.

In specific embodiments, adenoviral vectors of the invention will comprise substantially the entire adenoviral genome with the exception that are deletions of all or part of one or more of the following regions: the E1a region, the E1b region, and/or the E3 region. In further specific embodiments, non-adenoviral nucleic acid may be present in one or more of the E1a region, the E1b region, and/or the E3 region.

In particular embodiments, adenoviral vectors prepared by methods of the invention will contain at least one origin of replication and/or a selection marker which allows for amplification of the vector in prokaryotic cells, such as E. coli.

Adeno-associated viral vectors and Herpes viral vectors may be prepared by methods of the invention which are similar to those described above. Thus, the invention further provides methods for preparing such vectors, as well as vectors produced by these methods, uses of these vectors, and compositions comprising these vectors.

The invention further provides methods for preparing alphaviral vectors (e.g., Sindbis virus vectors, Semliki Forest virus vectors, Ross River virus vectors, Venezuelan equine encephalitis virus vectors, Western equine encephalitis virus vectors, Eastern equine encephalitis virus vectors, etc.), as well as alphaviral vectors prepared by such methods, methods employing these alphaviral vectors and compositions comprising these alphaviral vectors. In particular such embodiments, the invention includes methods for preparing alphaviral vectors by joining at least one nucleic acid segment which comprises alphaviral sequences to one or more other nucleic acid segments.

Specific examples of alphaviral vectors and nucleic acids which can be used to prepare alphaviral vectors are described in U.S. Pat. Nos. 5,739,026 and 6,224,879, the GibcoBRL's Instruction Manual No. 10179-018, “SFV Gene Expression System,” and Invitrogen's Sindbis Expression System manual, catalog no. K750-01 (version E), the entire disclosures of which are incorporated herein by reference. In specific embodiments, alphaviral vector sequences used in methods of the invention to prepare alphaviral vectors will comprise one or more of the following components: one or more packaging signals (which may or may not be of alphaviral origin), one or more subgenomic promoters, and/or nucleic acid encoding one or more non-structural protein (e.g., nsp1, nsp2, nsp3, nsp4, etc.).

Alphaviral vectors of the invention may be introduced into cells as DNA or RNA molecules. When DNA forms of such vectors are introduced into cells, expression control sequences (e.g., inducible, repressible or constitutive expression control sequences) may then be used to generate RNA molecules from which one or more non-structural proteins may be translated. In specific embodiments, these non-structural proteins will form an RNA-dependent RNA polymerase which will amplify RNA molecules corresponding to all or part of the transcript generated from the DNA form of the alphaviral vector. Thus, these non-structural proteins may catalyze the production of additional copies of RNA molecules from RNA templates, resulting in RNA amplification. Further, a nucleic acid segment for which high levels of expression is desired may be operably linked to a subgenomic promoter, thus resulting in the production of high levels of RNA corresponding to the nucleic acid segment.

In one exemplary embodiment, alphaviral vectors prepared by methods of the invention comprise DNA wherein an inducible promoter directs transcription of an RNA molecule which encodes nsp1, nsp2, nsp3, and nsp4 of a Sindbis virus and a Sindbis subgenomic promoter operatively linked to a nucleic acid segment which is not of Sindbis viral origin. The invention also provides alphaviral vectors prepared by methods of the invention, methods of using such alphaviral vectors, and compositions comprising such alphaviral vectors.

The invention further provides methods for joining nucleic acid segments wherein one or more of the nucleic acid segments contains one or more (e.g., one, two, three, four, etc.) viral packaging signal (e.g., one or more packaging signal derived from a virus referred to above). These packaging signals can be used to direct the packaging of nucleic acid molecules prepared by methods of the invention. One method for preparing packaged nucleic acid molecules is by the introduction or expression of nucleic acid molecules of the invention into packaging cell lines which express proteins suitable for the production of virus-like particles. The invention thus further includes packaged nucleic acid molecules of the invention, methods for preparing packaged nucleic acid molecules of the invention, and compositions comprising packaged nucleic acid molecules of the invention.

a. Introduction of Vectors

Methods for introducing the compositions, complexes, nucleic acid molecules and/or vectors of the invention into cells, tissues, organs or organisms as described herein will be familiar to those of ordinary skill in the art. For instance, the compositions, nucleic acid molecules and/or vectors of the invention may be introduced into cells, tissues, organs or organisms using well known techniques of infection, transduction, transfection, and transformation. The compositions, nucleic acid molecules and/or vectors of the invention may be introduced alone or in conjunction with other compositions, nucleic acid molecules and/or vectors. Alternatively, the compositions, nucleic acid molecules and/or vectors of the invention may be introduced into cells, tissues, organs or organisms as a precipitate, such as a calcium phosphate precipitate, or in a complex with a lipid. Electroporation also may be used to introduce the nucleic acid molecules and/or vectors of the invention into a host. Likewise, such molecules may be introduced into chemically competent cells such as E. coli. If the vector is a virus, it may be packaged in vitro or introduced into a packaging cell and the packaged virus may be transduced into cells. Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W. H. Freeman and Co., pp. 213-234 (1992), and Winnacker, E., From Genes to Clones, New York: VCH Publishers (1987), which are illustrative of the many laboratory manuals that detail these techniques and which are incorporated by reference herein in their entireties for their relevant disclosures.

A variety of reagents, compounds and compositions are useful for introducing the compositions, complexes, nucleic acid molecules and/or vectors of the invention into cells, tissues, organs or organisms, particularly via transfection. Examples of such suitable reagents, compounds and compositions include, but are not limited to, those shown in Table 4. In addition, calcium phosphate-mediated transfection can be employed (Sigma-Aldrich and Invitrogen Corporation, among others, provide transfection kits or systems using CaPO₄). For general reviews, see Hope et al., 1998. Cationic lipids, phosphatidylethanolamine and the intracellular delivery of polymeric, nucleic acid-based drugs (review). Mol Membr Biol 15:1-14; and Zabner J. 1997. Cationic lipids used in gene transfer. Adv Drug Deliv Rev 27:17-28. The agents listed can be used alone or in combination with other agents. Non-limiting exemplary formulations of multiple reagents are shown in Table 4.

TABLE 4
NON-LIMITING EXAMPLES OF TRANSFECTION
AGENTS

| TRANSFECTION | | | |
|---|---|---|---|
| AGENT | DESCRIPTION | PATENTS AND/OR CITATIONS | AVAILABLE FROM |
| BMOP | N-(2-bromoethyl)-N,N-dimethyl- | | |
| | 2,3-bis(9-octadecenyloxy)-propana | | |
| | minimun bromide) | | |
| BMOP:DOPE | 1:1 (wt/wt) formulation of N-(2- | Poult Sci 1997 Jun; 76(6): 882-6. Transfection of | |
| | bromoethyl)-N,N-dimethyl-2,3- | avian LMH-2A hepatoma cells with cationic lipids. | |
| | bis(9-octadecenyloxy)-propana | Walzem R L, Hickman M A, German J B, Hansen R J. | |
| | minimun bromide) (BMOP) and | | |
| | DOPE | | |
| Cationic | Cationic polysaccharides | Published U.S. patent application 2002/0146826 | |
| polysaccharides | | | |
| CellFECTIN ® | 1:1.5 (M/M) formulation of N, NI, | U.S. Pat. No. 5,674,908, 5,834,439 and 6,110,916 | Invitrogen (LTI) |
| | NII, NIII-tetramethyl-N, NI, NII, | | |
| | NIII-tetrapalmitylspermine (TM- | | |
| | TPS) and dioleoyl | | |
| | phosphatidylethanolamine (DOPE) | | |
| CLONfectin ™ | N-t-butyl-N′-tetradecyl-3- | Ruysschaert, J. M., et al. (1994) Biochem. Biophys. | BD Biosciences Clontech |
| | tetradecyl-aminopropion-amidine | Res. Comm. 203: 1622-1628 | |
| CTAB:DOPE | formulation of cetyltrimethyl- | | |
| | ammonium bromide (CATB) and | | |
| | dioleoylphosphatidylethanol-amine | | |
| | (DOPE) | | |
| Cytofectene | proprietary cationic lipid and DOPE | | Bio-Rad Laboratories |
| Cytofectin GSV | 2:1 (M/M) formulation of cytofectin | | (*Cytofectin GS |
| | GS* and dioleoyl phosphatidyl- | | corresponds to Gilead |
| | ethanolamine (DOPE) | | Sciences' GS 3815) |
| DC-Cholesterol (DC- | 3,β-N,(N′,N′- | | |
| Chol) | dimethylaminoethane)-carbamoyl] | | |
| | cholesterol | | |
| DC-Chol:DOPE | formulation of 3,β-N,(N′,N′- | Gao et al., Biochim. Biophys. Res. Comm. 179: 280-285 | |
| | dimethylaminoethane)-carbamoly] | (1991) | |
| | cholesterol (DC-Chol) and | | |
| | dioleoyl phosphatidyl-ethanolamine | | |
| | (DOPE) | | |
| DC-6-14 | O,O′-Ditetradecanoyl-N-(alpha- | Hum Gene Ther 1999 Apr 10; 10(6): 947-55. | |
| | trimethylammonioacetyl)diethanolamine | Development of novel cationic liposomes for | |
| | chloride | efficient gene transfer into peritoneal disseminated | |
| | | tumor. Kikuchi A, Aoki Y, Sugaya S, Serikawa T, | |
| | | Takakuwa K, Tanaka K, Suzuki N, Kikuchi H. | |
| DCPE | Dicaproylphosphtidylethanol-amine | | |
| DDPES | Dipalmitoylphosphatidyl- | Behr et al. 1989. Efficient gene transfer into | |
| | ethanolamine 5- | mammalian primary endocrine cells with | |
| | carboxyspermylamide | lipopolyamine-coated DNA. Proc. Natl. Acad. Sci. | |
| | | USA 86: 6982-6986; EPO Publication 0 394 111 | |
| DDAB | didoceyl methylammonium | | |
| | bromide | | |
| Dextran and dextran | DEAE-Dextran; Dextran sulfate | J Biol Chem. 2002. 277: 30208-30218. Efficiency of | |
| derivatives or | | protein transduction is cell type-dependent and is | |
| conjugates | | enhanced by dextran sulfate. Mai J C, Shen H, | |
| | | Watkins S C, Cheng T, Robbins P D. | |
| Diquaternary | (examples:) N,N′-dioleyl- | Bioconjug Chem 2001 Mar-Apr; 12(2): 258-63. | Vical |
| ammonium salts | N,N,N′,N′-tetramethyl-1,2- | Diquaternary ammonium compounds as transfection | |
| | ethanediamine (TmedEce), N,N′- | agents. Rosenzweig H S, Rakhmanova V A, | |
| | dioleyl-N,N,N′,N′-tetramethyl-1,3- | MacDonald R C.; U.S. Pat. No. 5,994,317 | |
| | propanediamine (PropEce), N,N′- | | |
| | dioleyl-N,N,N′,N′-tetramethyl-1,6- | | |
| | hexanediamine (HexEce), and their | | |
| | corresponding N,N′-dicetyl | | |
| | saturated analogues (TmedAce, | | |
| | PropAce and HexAce) | | |
| DLRIE | dilauryl oxypropyl-3- | Ann N Y Acad Sci 1995 Nov 27; 772: 126-39. | Vical |
| | dimethylhydroxy ethylammonium | Improved cationic lipid formulations for in vivo | |
| | bromide | gene therapy. Felgner P L, Tsai Y J, Sukhu L, | |
| | | Wheeler C J, Manthorpe M, Marshall J, Cheng S H. | |
| DMAP | 4-dimethylaminopyridine | | |
| DMPE | Dimyristoylphospatidylethanolamine | | |
| DMRIE | N-[1-(2,3-dimyristyloxy)propyl]- | Biochim Biophys Acta 1996 Jul 24; 1312(3): 186-96. | |
| | N,N-dimethyl-N-(2-hydroxyethyl) | Human immunodeficiency virus type-1 (HIV-1) | |
| | ammonium bromide | infection increases the sensitivity of macrophages | |
| | | and THP-1 cells to cytotoxicity by cationic | |
| | | liposomes. Konopka K, Pretzer E, Felgner P L, | |
| | | Duzgunes N. | |
| DMRIE-C | 1:1 formulation of N-[1-(2,3- | U.S. Pat. No. 5,459,127 and 5,264,618, to Felgner, et | Invitrogen (LTI) |
| | dimyristyloxy)propyl]-N,N- | al. (Vical) | |
| | dimethyl-N-(2-hydroxyethyl) | | |
| | ammonium bromide (DMRIE) and | | |
| | cholesterol | | |
| DMRIE:DOPE | formulation of 1,2- | Hum Gene Ther 1993 Dec; 4(6): 781-8. Safety and | |
| | dimyristyloxypropyl-3-dimethyl- | short-term toxicity of a novel cationic lipid | |
| | hydroxyethyl ammonium bromide | formulation for human gene therapy. San H, Yang Z Y, | |
| | and dioleoyl phosphatidyl- | Pompili V J, Jaffe M L, Plautz G E, Xu L, | |
| | ethanolamine (DOPE) | Felgner J H, Wheeler C J, Felgner P L, Gao X, et al. | |
| DOEPC | dioleoylethylphosphocholine | | |
| DOHME | N-[1-(2,3-dioleoyloxy)propyl]-N- | | |
| | [1-(2-hydroxyethyl)]-N,N- | | |
| | dimethylammonium iodide | | |
| DOPC | dioleoylphosphatidylcholine | | |
| DOPC:DOPS | 1:1 (wt %) formulation of DOPC | | Avanti |
| | (dioleoylphosphatidylcholine) and | | |
| | DOPS | | |
| DOSPA | 2,3-dioleoyloxy-N-[2- | | |
| | (sperminecarboxamidoethyl]-N,N- | | |
| | di-met-hyl-1-propanaminium | | |
| | trifluoroacetate | | |
| DOSPA:DOPE | Formulation of 2,3-dioleoyloxy-N- | J Gene Med 2001 Jan-Feb; 3(1): 82-90. Cationic | |
| | [2-(sperminecarboxamidoethyl]- | liposome-mediated gene transfer to rat salivary | |
| | N,N-di-met-hyl-1-propanaminium | epithelial cells in vitro and in vivo. Baccaglini L, | |
| | trifluoroacetate (DOSPA) and | Shamsul Hoque A T, Wellner R B, Goldsmith C M, | |
| | dioleoyl phosphatidyl-ethanolamine | Redman R S, Sankar V, Kingman A, Barnhart K M, | |
| | (DOPE) | Wheeler C J, Baum B J. | |
| DOSPER | 1,3-Di-Oleoyloxy-2-(6-Carboxy- | Buchberger et al., 1996. DOSPER liposomal | Roche |
| | spermyl)-propylamid | transfection reagent: a reagent with unique | |
| | | transfection properties. Biochemica 2: 7-10. | |
| DOTAP | N-[1-(2,3-dioleoyloxy)propyl]- | | |
| | N,N,N-trimethyl-ammonium | | |
| | methylsulfate | | |
| DOTMA | N-[1-(2,3-dioleyloxy)propyl]-n,n,n- | | |
| | trimethylammoniumchloride | | |
| DPEPC | Dipalmitoylethylphosphatidyl- | | |
| | choline | | |
| Effectene | (non-liposomal lipid formulation | Histochem Cell Biol 2001 Jan; 115(1): 41-7. Long- | Qiagen |
| | used in conjunction with a special | term expression of foreign genes in normal human | |
| | DNA-condensing enhancer and | epidermal keratinocytes after transfection with | |
| | optimized buffer) | lipid/DNA complexes. Zellmer S, Gaunitz F, | |
| | | Salvetter J, Surovoy A, Reissig D, Gebhardt R. | |
| ExGen 500 | Apyrogenic solution of linear | Ferrari S., Moro E., Pettenazzo A., Behr J. P., | Fermetas |
| | 22 kDa polyethylenimine (PEI) in | Zacchello F., Scarpa M., ExGen 500 is an efficient | |
| | water | vector for gene delivery to lung epithelial cells in | |
| | | vitro and in vivo, Gene Ther, Oct; 4(10): 1100-1106, | |
| | | 1997 | |
| FuGENE 6 | (proprietary formulation) | J Neurosci Methods 1999 Oct 15; 92(1-2): 145-52. | Roche |
| | | Improved lipid-mediated gene transfer in C6 glioma | |
| | | cells and primary glial cells using FuGene. | |
| | | Wiesenhofer B, Kaufmann W A, Humpel C. | |
| GAP-DLRIE:DOPE | N-(3-aminopropyl)-N,N-dimethyl- | Hum Gene Ther 1996 Oct 1; 7(15): 1803-12. A new | |
| | 2,3-bis(dodecyloxy)-1- | cationic liposome DNA complex enhances the | |
| | propaniminium bromide/dioleyl | efficiency of arterial gene transfer in vivo. Stephan D J, | |
| | phosphatidylethanolamine | Yang Z Y, San H, Simari R D, Wheeler C J, | |
| | | Felgner P L, Gordon D, Nabel G J, Nabel E G | |
| GeneJammer | Proprietary polyamine | | Wako, USA |
| GeneJuice | Proprietary polyamine | | Novagen |
| GeneLimo | Proprietary liposomal formulations | | CPG, Inc. |
| | of polycationic lipids and a neutral, | | |
| | non-transfecting lipid compound | | |
| GeneSHUTTLE ™ | Novel extruded DOTAP and | | |
| | cholesterol (DOTAP: Chol) | | |
| | formulation | | |
| Genetransfer | Liposome-mediated | | Strategene |
| Genetransfer | | | Wako Pure Chemical |
| | | | (Japan) |
| GS 2888 cytofectin | | Proc Natl Acad Sci USA 1996 Apr 16; 93(8): 3176-81. | Gilead Sciences |
| | | A serum-resistant cytofectin for cellular delivery | |
| | | of antisense oligodeoxynucleotides and plasmid | |
| | | DNA. Lewis J G, Lin K Y, Kothavale A, Flanagan W M, | |
| | | Matteucci M D, DePrince R B, Mook R A Jr, | |
| | | Hendren R W, Wagner R W. | |
| Lipofectin ® | 1:1 (w/w) formulation of N-(1-2,3- | U.S. Pat. No. 4,897,355; 5,208,066; and 5,550,289. | Invitrogen (LTI) |
| | dioleyloxypropyl)-N,N,N- | | |
| | triethylammonium (DOTMA) and | | |
| | dioleylphosphatidylethanolamine | | |
| | (DOPE) | | |
| LipofectACE ™ | 1:2.5 (w/w) formulation of dimethyl | | Invitrogen (LTI) |
| | dioctadecylammonium bromide | | |
| | (DDAB) and dioleoyl | | |
| | phosphatidylethanolamine (DOPE) | | |
| LipofectAMINE ™ | 3:1 (w/w) formulation of 2,3- | U.S. Pat. No. 5,334,761; and U.S. Pat. No. 5,459,127 | Invitrogen (LTI) |
| | dioleyloxy-N- | and 5,264,618, to Felgner, et al. (Vical) | |
| | [2(sperminecarboxamido)ethyl]- | | |
| | N,N-dimethyl-1-propanaminium | | |
| | trifluoroacetate (DOSPA) and | | |
| | dioleoyl phosphatidylethanolamine | | |
| | (DOPE) | | |
| LipofectAMINE ™ | (proprietary formulation) | | Invitrogen (LTI) |
| 2000 | | | |
| LipofectAMINE | PLUS (proprietary formulation) and | U.S. Pat. No. 5,736,392 and 6,051,429 | Invitrogen (LTI) |
| PLUS ™ | LipofectAMINE ™ | | |
| LipoTAXI ® | (proprietary formulation) | Madry H, Trippel S B. Efficient lipid-mediated gene | Stratagene |
| | | transfer to articular chondrocytes. Gene Ther. 2000 | |
| | | Feb; 7(4): 286-91. | |
| monocationic | (examples:) 1-deoxy-1- | J Med Chem 2001 Nov 22; 44(24): 4176-85. Design, | |
| transfection lipids | [dihexadecyl(methyl)ammonio]-D- | synthesis, and transfection biology of novel cationic | |
| | xylitol; 1-deoxy-1- | glycolipids for use in liposomal gene delivery. | |
| | [methyl(ditetradecyl)ammonio]-D- | Banerjee R, Mahidhar Y V, Chaudhuri A, Gopal V, | |
| | arabinitol; 1-deoxy-1- | Rao N M. | |
| | [dihexadecyl(methyl)ammonio]-D- | | |
| | arabinitol; 1-deoxy-1- | | |
| | [methyl(dioctadecyl)ammonio]-D- | | |
| | arabinitol | | |
| O-Chol | 3 beta[l-ornithinamide-carbamoyl] | Gene Ther 2002 Jul; 9(13): 859-66. Intraperitoneal | |
| | cholesterol | gene delivery mediated by a novel cationic liposome | |
| | | in a peritoneal disseminated ovarian cancer model. | |
| | | Lee M J, Cho S S, You J R, Lee Y, Kang B D, Choi J S, | |
| | | Park J W, Suh Y L, Kim J A, Kim D K, Park J S. | |
| OliogfectAMINE ™ | (proprietary formulation) | | Invitrogen (LTI) |
| Piperazine based | Piperazine based amphilic cationic | U.S. Pat. No. 5,861,397 and 6,022,874 | Vical |
| amphilic cationic | lipids | | |
| lipids | | | |
| PolyFect | (activated-dendrimer molecules | | Qiagen |
| | with a defined spherical | | |
| | architecture) | | |
| Protamine | Protamine mixture prepared from, | Gene Ther 1997 Sep; 4(9): 961-8. Protamine sulfate | Sigma |
| | e.g., salmon, salt herring, etc.; can | enhances lipid-mediated gene transfer. Sorgi F L, | |
| | be supplied as, e.g., a sulfate or | Bhattacharya S, Huang L. | |
| | phosphate. | | |
| SuperFect | (activated-dendrimer molecules | Tang, M. X., Redemann, C. T., and Szoka, Jr., F. C. | Qiagen |
| | with a defined spherical | (1996) In vitro gene delivery by degraded | |
| | architecture) | polyamidoamine dendrimers. Bioconjugate Chem. | |
| | | 7: 703; published PCT applications WO 93/19768 | |
| | | and WO 95/02397 | |
| Tfx ™ | N,N,N′,N′-tetramethyl-N,N′-bis(2- | | Promega |
| | hydroxyethyl)-2,3-di(oleoyloxy)- | | |
| | 1,4-butanediammonium iodide] and | | |
| | DOPE | | |
| TransFAST ™ | N,N [bis (2-hydroxyethyl)-N- | | Promega |
| | methyl-N-[2,3-di(tetradecanoyloxy) | | |
| | propyl] ammonium iodide and | | |
| | DOPE | | |
| TransfectAce | | | Invitrogen (LTI) |
| TRANSFECTAM ™ | 5-carboxylspermylglycine | Behr et al. 1989. _. Proc. Natl. Acad. Sci. USA | Promega |
| | dioctadecylamide (DOGS) | 86: 6982-6986; EPO Publication 0394 111 | |
| TransIT ®-LT1, | Proprietary combination of a | | Panvera, Mirus |
| TransIT ®-LT2 and | nontoxic cellular protein & a | | |
| various other | proprietary polyamine | | |
| TransIT ® products | | | |
| TransMessenger | (lipid-based formulation that is used | | Qiagen |
| | in conjunction with a specific RNA- | | |
| | condensing enhancer and an | | |
| | optimized buffer; particularly useful | | |
| | for mRNA transfection) | | |
| Vectamidine | 3-tetradecylamino-N-tert-butyl-N′- | FEBS Lett 1997 Sep 8; 414(2): 187-92. The role of | |
| | tetradecylpropionamidine (a.k.a. | endosome destabilizing activity in the gene transfer | |
| | diC14-amidine) | process mediated by cationic lipids. El Ouahabi A, | |
| | | Thiry M, Pector V, Fuks R, Ruysschaert J M, | |
| | | Vandenbranden M. | |
| X-tremeGENE Q2 | (proprietary formulation) | | Roche Molecular |
| | | | Biochemicals |

b. Release of Nucleic Acids Intracellularly

Once internatlized into a cell (typically via endocytosis), transfected nucleic acids are usually sequestered within lipid membrane-enclosed vesicles (including endosomes, as well as components of the endoplasmic reticulum (ER) and/or Golgi apparatus). The release of nucleic acids into the cytosol from endosomes, the ER or the Golgi enhances transfection. Endosomal disrupting agents can be used in the context of the invention and are defined herein as agents that cause or enhance the release of nucleic acids into the cytosol. Endosomal disrupting agents can act, by way of non-limiting example, by disrupting membranes of endosomes, the ER, the Golgi apparatus and/or other membranes; blocking or reducing endosome fusion to lysosomes; and/or altering, preferably raising, the pH of endosomes. The pH of an endosome is generally lower than that of the cytosol by one to two pH units. This pH gradient can be exploited for cellular delivery using agents that disrupt lipid bilayer membranes at pH 6.5 and below (Asokan A, Cho M J. 2002.

Exploitation of intracellular pH gradients in the cellular delivery of macromolecules. J Pharm Sci 91:903-913).

Membrane-disruptive pH-sensitive synthetic polymers have been described and include by way of non-limiting example poly(amidoamine)s (PAAs) (Pattrick et al., 2001. Poly(amidoamine)-mediated intracytoplasmic delivery of ricin A-chain and gelonin. J Control Release 77:225-32; U.S. Pat. No. 6,413,941); poly(propylacrylic acid) (PPAA) (Kyriakides et al., 2002. pH-sensitive polymers that enhance intracellular drug delivery in vivo. J Control Release78:295-303); and poly(ethyl acrylic acid) (PEAAc) (Murthy et al., 1999. The design and synthesis of polymers for eukaryotic membrane disruption. J Control Release 61:137-43).

The capacity of adenoviruses to disrupt endosomes as part of their entry mechanism has been exploited in various gene delivery systems. See Zatloukal et al., 1994. Genetic modification of cells by receptor-mediated adenovirus-augmented gene delivery: a new approach for immunotherapy of cancer. Verh Dtsch Ges Pathol 78:171-6; Michael et al., 1993. Binding-incompetent adenovirus facilitates molecular conjugate-mediated gene transfer by the receptor-mediated endocytosis pathway. J Biol Chem 268:6866-9; Cotten et al., 1992. High-efficiency receptor-mediated delivery of small and large (48 kilobase gene constructs using the endosome-disruption activity of defective or chemically inactivated adenovirus particles. Proc Natl Acad Sci USA 89:6094-8. Curiel et al., 1991. Adenovirus enhancement of transferrin-polylysine-mediated gene delivery. Proc Natl Acad Sci USA 88:8850-4. In addition to human adenovirus, other adenoviruses, including by way of non-limiting example chicken adenovirus, can be used as endosomal disrupting agents (Cotten et al., 1993. Chicken adenovirus (CELO virus) particles augment receptor-mediated DNA delivery to mammalian cells and yield exceptional levels of stable transformants. J Virol 67:3777-85).

Some cationic lipid transfection reagents, such as vectamidine and DMRIE-C, may have inherent endosomal disrupting properties. See El Ouahabi et al., 1997. The role of endosome destabilizing activity in the gene transfer process mediated by cationic lipids. FEBS Lett 414:187-92. Moreover, cationic lipids that are acid-labile have been described (Boomer et al., 2002. Formation of plasmid-based transfection complexes with an acid-labile cationic lipid: characterization of in vitro and in vivo gene transfer. Pharm Res 19:1292-1301; Wetzer et al., 2001. Reducible cationic lipids for gene transfer. Biochem J 356:747-756).

Other endosome disrupting agents include viral fusogenic peptides, including without limitation influenza virus hemagglutinin fusogenic peptides (Bongartz et al., 1994. Improved biological activity of antisense oligonucleotides conjugated to a fusogenic peptide. Nucleic Acids Res 22:4681-4688) and synthetic derivatives thereof (Plank et al., 1994. The influence of endosome-disruptive peptides on gene transfer using synthetic virus-like gene transfer systems. J. Biol.

Chem. 269:12918-12924. These peptides are thought to change conformation at acidic pH and destabilize endosomal membranes.

The ricin A chain, which is capable of penetrating out of endosomes and into the cytosol, can be attached to a nucleic acid or protein to in order to effect the endosomal release thereof (Beaumell et al., 1993. ATP-dependent translocation of ricin across the membrane of purified endosomes J. Biol. Chem. 268:23661-23669).

Agents that alter the pH of endosomes can be used to practice the invention. Lysosomotropic amines are generally thought to effect of raising the pH of endosomes. Such agents include without limitation ammonium chloride, 4-aminoquinolines (e.g., chloroquine, amodiaquine), 8-aminoquinolines (e.g., primaquine and WR242511), pyrimethamine, quinacrine, quinine and quinidine (Tsiang H, Superti F. Ammonium chloride and chloroquine inhibit rabies virus infection in neuroblastoma cells. Brief report. Arch Virol 81:377-382; Deshpande et al., 1997. Efficacy of certain quinolines as pharmacological antagonists in botulinum neurotoxin poisoning. Toxicon 35:433-445).

C. Artificial Chromosomes

The nucleic acid molecules used in the compositions, complexes and methods of the present invention may alternatively be in the form of artificial chromosomes (ACs). An AC is a DNA molecule that comprises, at a minimum, at least one origin of DNA replication (ori), one or more telomeres and a centromere. Each ori is preferably derived from a genomic chromosome, so that replication of the AC is coordinated with cellular DNA replication. The telomeres are elements that preserve the terminal sequences of chromosomes for any number of rounds of replication and cell division. The centromere mediates proper segregation of the AC through each cell division (Willard H F. Centromeres: the missing link in the development of human artificial chromosomes. Curr Opin Genet Dev 8:219-225, 1998).

Ideally, ACs are stably maintained and are properly segregated during both mitosis and meiosis. Generally, an AC contains a segment of cloned DNA, and is usually more stable the larger the piece of cloned DNA. It is possible to engineer ACs to improve or add functions (Grimes B, Cooke H. Engineering mammalian chromosomes. Hum Mol Genet 7:1635-1640, 1998; Saffery R, Choo KH. Strategies for engineering human chromosomes with therapeutic potential. J Gene Med 4:5-13, 2002).

Bacterial and yeast artificial chromosomes (BACs and YACs, respectively) have been described. BACs and YACs are reviewed in Shizuya H, Kouros-Mehr H. The development and applications of the bacterial artificial chromosome cloning system. Keio J Med 50:26-30, 2001; and Fabb S A, Ragoussis J. Yeast artificial chromosome vectors. Mol Cell Biol Hum Dis Ser 5:104-124, 1995; Anand R. Yeast artificial chromosomes (YACs) and the analysis of complex genomes, Trends Biotechnol 10:35-40, 1992.

Mammalian artificial chromosomes (MACs) have been prepared and may be used as vectors for somatic gene therapy. See Brown W R. Mammalian artificial chromosomes. Curr Opin Genet Dev 2:479-486, 1992; Huxley C. Mammalian artificial chromosomes and chromosome transgenics. Trends Genet 13:345-347, 1997; Ascenzioni F, Donini P, Lipps H J. Mammalian artificial chromosomes—vectors for somatic gene therapy. Cancer Lett 118:135-142, 1997; Vos J M. Mammalian artificial chromosomes as tools for gene therapy. Curr Opin Genet Dev 8:351-359, 1998; and Vos J M. Therapeutic mammalian artificial episomal chromosomes. Curr Opin Mol Ther 1:204-215, 1999.

Human artificial chromosomes (HACs) have been described (Henning K A, Novotny E A, Compton S T, Guan X Y, Liu P P, Ashlock M A. Human artificial chromosomes generated by modification of a yeast artificial chromosome containing both human alpha satellite and single-copy DNA sequences. Proc Natl Acad Sci U S A. 96:592-597, 1999; Larin Z, Mejia J E. Advances in human artificial chromosome technology. Trends Genet 18:313-319, 2002). HACs include but are not limited to satellite DNA-based artificial chromosomes (SATACs). SATACs have been made by mixing human telomeric DNA, genomic DNA, and arrays of repetitive α-satellite DNA having centromeric activity (Hadlaczky G. Satellite DNA-based artificial chromosomes for use in gene therapy. Curr Opin Mol Ther. 3:125-132, 2001).

In addition to gene therapy, ACs have been used to stably clone large pieces of DNA in a variety of cell types (Schlessinger D, Nagaraja R. Impact and implications of yeast and human artificial chromosomes. Ann Med 30:186-191, 1998; Monaco A P, Larin Z. YACs, BACs, PACs and MACs: artificial chromosomes as research tools. Trends Biotechnol. 12:280-286, 1994). In addition, ACs can be also be used in transgenic animal technologies to introduce large transgenes in animals, especially human transgenes in mouse models of human genetic diseases. See Giraldo P, Montoliu L. Size matters: use of YACs, BACs and PACs in transgenic animals. Transgenic Res 10:83-103, 2001; Jakobovits A, Lamb B T, Peterson K R. Production of transgenic mice with yeast artificial chromosomes. Methods Mol Biol 136:435-453, 2000; Lamb B T, Gearhart J D. YAC transgenics and the study of genetics and human disease. Curr Opin Genet Dev 5:342-348, 1995; Jakobovits A. YAC vectors. Humanizing the mouse genome. Curr Biol 4:761-763, 1994; Huxley C. Transfer of YACs to mammalian cells and transgenic mice. Genet Eng (N Y) 16:65-91, 1994; Huxley C, Gnirke A. Transfer of yeast artificial chromosomes from yeast to mammalian cells. Bioessays 13:545-550, 1991; and Heintz N. BAC to the future: the use of bac transgenic mice for neuroscience research. Nat Rev Neurosci 2:861-870, 2001.

D. Peptide Nucleic Acids (PNAs)

The nucleic acid molecules used in the compositions, complexes and methods of the present invention may alternatively be in the form of peptide nucleic acids (PNAs). PNAs are analogs of nucleic acid molecules in which the backbone is a pseudopeptide rather than a sugar. Like DNA and RNA, a PNA molecule binds single-stranded nucleic acid having a reverse complementary sequence; however, the neutral backbone of PNAs can result in stronger binding and greater specificity. For a review, see Corey D R. Peptide nucleic acids: expanding the scope of nucleic acid recognition. Trends Biotechnol 15:224-229, 1997. The synthesis of PNAs is reviewed by Hyrup et al. (Peptide nucleic acids (PNA): synthesis, properties and potential applications. Bioorg Med Chem. 4:5-23, 1996). For exemplary protocols for making and using PNAs, see Peptide Nucleic Acids: Protocols and Applications, Nielsen, P. E. and Egholm, M., eds. Horizon Scientific Press, Norfolk, U.K. 1999. PNAs can be prepared according to methods known in the art or purchased commercially from, e.g., Monomer Sciences Inc. (New Market, Ala., U.S.) and Dalton Chemical Laboratories Inc. (Toronto, ON, Canada). Methods for attaching fluorescent moieties to PNA have been described. See, e.g., Murakami et al., A novel method for detecting HIV-1 by non-radioactive in situ hybridization: application of a peptide nucleic acid probe and catalysed signal amplification. Pathol 194:130-135, 2001.

VI. Fluorescent Molecules and Moieties

In certain embodiments, the compositions and complexes of the invention will comprise one or more marker or activation molecules or moieties, such as one or more molecules or moieties that are linked to, complexed with, or comprise, one or more fluorophores. Contemplated by this aspect of the invention are compositions in which the one or more fluorophores is linked (e.g., bound covalently or ionically) to one or more components of the compositions of the invention (e.g., fluorescently tagged nucleic acid molecules, nucleotides, proteins, peptides, and the like). Also contemplated by this aspect of the invention are compositions in which the one or more fluorophores is contained separately within the composition, without necessarily being directly linked to one or more of the other components within the composition.

A. Fluorophores

For the purpose of the present invention, a fluorophore can be a substance which itself fluoresces, or a substance that fluoresces in particular situations (e.g., when in proximity to another fluorophore, as occurs in FRET). The term “fluorophore” or “fluor” is meant to encompass fluorescent moieties that are covalently linked to another molecule, fluorescent molecules that are non-covalently attached to another molecule, as well as free fluorescent molecules. Molecules that become fluorescent only after attachment to another molecule, such as a peptide or nucleic acid, are also within the scope of the invention.

In principal, any fluorophore now known, or later discovered, can be used in accordance with the methods, compositions and kits of the present invention. In certain embodiments, fluorophores suitable for use in the present invention include those that are excitable at, and/or emit fluorescence at, a wavelength falling within the range of wavelengths from about 200 nm to about 800 nm; from about 250 nm to about 800 nm; from about 250 nm to about 750 nm; from about 300 nm to about 700 nm; from about 350 nm to about 650 nm; from about 400 nm to about 600 nm; from about 450 nm to about 600 nm; from about 450 nm to about 580 nm; from about 450 nm to about 575 nm; from about 450 nm to about 570 nm; from about 500 nm to about 600 nm; from about 500 nm to about 590 nm; from about 500 nm to about 580 nm; from about 500 nm to about 575 nm; from about 500 nm to about 570 nm; and the like. As one of ordinary skill will readily appreciate, any fluorophore with an excitation maximum and an emission maximum within the recited ranges is suitable for use in accordance with the present invention, whether or not the actual, specific excitation and emission maxima for that given fluorophore are specifically set forth above.

In view of the availability of an array of appropriate compounds, it is well within the capabilities of one skilled in the art to choose a reactive fluorescent molecule or set of molecules that is appropriate to the practice of the present invention, given the above-noted guidelines for excitation and emission maxima. Many appropriate fluorophores are commercially available from sources such as Molecular Probes Inc. (Eugene, Oreg.).

Many of these methods are quite appropriate for use in preparing the various compounds required to practice the present invention. One skilled in the art will be able, without undue experimentation, to choose a suitable method for preparing a desired fluorescently labeled nucleic acid, oligonucleotide or the like. See, for example, Protocols for Oligonucleotide Conjugates, Vol. 26 of Methods in Molecular Biology, Agrawal, ed., Humana Press, Totowa, New Jersey (1994). Additionally, as the art of organic synthesis, particularly in the area of nucleic acid chemistry, continues to expand in scope new methods will be developed which are equally as suitable as those now known. The following discussion is offered as representative of the array of compounds and techniques that can be used to modify nucleic acids. Methods useful in conjunction with the present invention are not to be construed as limited by this discussion.

Fluorescent moieties and molecules useful in practicing the present invention include but are not limited to derivatives of fluorescein, rhodamine, coumarin, dimethylaminonaph-thalene sulfonic acid (dansyl), pyrene, anthracene, nitrobenz-oxadiazole (NBD), acridine and dipyrrometheneboron difluoride. More specifically, non-limiting examples of fluorescent moieties and molecules useful in practicing the present invention include, but are not limited to:

carbocyanine, dicarbocyanine, merocyanine and other cyanine dyes (e.g., CyDye™ fluorophores, such as Cy3, Cy3.5, Cy5, Cy5.5 and Cy7 from Pharmacia). These dyes have a maximum fluorescence at a variety of wavelengths: green (506 nm and 520 nm), green-yellow (540 nm), orange (570 nm), scarlet (596 nm), far-red (670 nm), and near infrared (694 nm and 767 nm);

coumarin and its derivatives (e.g., 7-amino-4-methylcoumarin, aminocoumarin and hydroxycoumarin);

BODIPY dyes (e.g., BODIPY FL, BODIPY 630/650, BODIPY 650/665, BODIPY TMR);

fluorescein and its derivatives (e.g., fluorescein isothiocyanate);

rhodamine dyes (e.g. rhodamine green, rhodamine red, tetramethylrhodamine, rhodamine 6G and Lissamine rhodamine B);

Alexa dyes (e.g., Alexa Fluor-350, -430, -488, -532, -546, -568, -594, -663 and -660, from Molecular Probes);

fluorescent energy transfer dyes (e.g., thiazole orange-ethidium heterodimer, TOTAB, etc.);

proteins with luminescent properties, e.g.: green fluorescent protein (GFP) and mutants and variants thereof, including by way of non-limiting example fluorescent proteins having altered wavelengths (e.g., YFP, RFP, etc.). See Chiesa et al. (2001). Recombinant aequorin and green fluorescent protein as valuable tools in the study of cell signalling. Biochem J. 355:1-12; Sacchetti et al. (2000). The molecular determinants of the efficiency of green fluorescent protein mutants. Histol Histopathol. 15:101-107; Larrick et al. (1995). Green fluorescent protein: untapped potential in immunotechnology. Immunotechnology 1:83-86);

aequorin and mutants and variants thereof;

DsRed protein (Baird et al., 2000. Biochemistry, mutagenesis, and oligomerization of DsRed, a red fluorescent protein from coral. Proc Natl Acad Sci USA 97:11984-9), and mutants and variants thereof (see Verkhusha et al., 2001. An enhanced mutant of red fluorescent protein DsRed for double labeling and developmental timer of neural fiber bundle formation. J Biol Chem 276:29621-4; Bevis B J, Glick BS., 2002. Rapidly maturing variants of the Discosoma red fluorescent protein (DsRed). Nat Biotechnol 20:83-87; Terskikh et al., 2002. Analysis of DsRed Mutants. Space around the fluorophore accelerates fluorescence development. J Biol Chem 277:7633-6; Campbell et al., 2002. A monomeric red fluorescent protein. Proc Natl Acad Sci USA 99:7877-82; and Knop et al., 2002. Improved version of the red fluorescent protein (drFP583/DsRed/RFP). Biotechniques 33:592, 594, 596-598); and

other fluors, e.g., 6-FAM, HEX, TET, F12-dUTP, L5-dCTP, 8-anilino-1-napthalene sulfonate, pyrene, ethenoadenosine, ethidium bromide prollavine monosemicarbazide, p-terphenyl, 2,5-diphenyl-1,3,4-oxadiazole, 2,5-diphenyloxazole, p-bis[2-(5-phenyloxazolyl)]benzene, 1,4-bis-2-(4-methyl-5-phenyloxazolyl)-benzene, lanthanide chelates, Pacific blue, Cascade blue, Cascade Yellow, Oregon Green, Marina Blue, Texas Red, phycoerythrin, eosins and erythrosins;

as well as derivatives of any of the preceding molecules and moieties. Fluorophores, and kits for attaching fluorophores to nucleic acids and peptides, are commercially available from, e.g., Molecular Probes (Eugene, Oreg.) and Sigma/Aldrich (St. Louis, Mo.).

B. Fluorescent Oligonucleotides and Other Nucleic Acids

Fluorescent moieties useful in practicing the present invention can be attached to any location on a nucleic acid, including sites on the base segment and sites on the sugar segment. Thus, the fluorophore is covalently attached to a nucleic acid at a position selected from the group consisting of the 3′-terminus, the 5′-terminus, an internal position and combinations thereof. See, generally, Goodchild, Bioconjug. Chem. 1:165-187 (1990). Although any suitable fluorophore can be associated with an oligonucleotide, some of the more commonly used ones are fluorescein, tetramethylrhodamine, Texas Red and Lissamine rhodamine B.

A number of techniques have been developed for converting specific constituents of DNA and RNA strands into fluorescent adducts. For a review, see, Leonard and Tolman, in “Chemistry, Biology and Clinical Uses of Nucleoside Analogs,” A. Bloch, ed., Ann. N.Y Acad. Sci. 255:43-58 (1975).

Chemical methods are available to introduce fluorescence into specific nucleic acid bases. For example, reaction of chloracetaldehyde with adenosine and cytidine yields fluorescent products. The reaction can be controlled with respect to which of the two bases is derivatized by manipulating the pH of the reaction mixture; the reaction at 37° C. proceeds rapidly at the optimum pH of 4.5 for adenosine and 3.5 for cytidine (Barrio et al., Biochem. Biophys. Res. Commun. 46:597-604, 1972). This reaction is also useful for rendering fluorescent the deoxyribosyl derivatives of these bases (Kochetkov et al., Dokl. Akad. Nauk. SSSR C 213:1327-1330, 1973).

DNA and RNA can be modified by reacting their cytidine residues with sodium bisulfite to form sulfonate intermediates that are then coupled to reactive nitrogen compounds such as hydrazides or amines (Viscidi et al J. Clin. Microbiol. 23:311, 1986; and Draper and Gold, Biochemistry 19:1774, 1980). RNA can also be labeled at the 3′ terminus by selective oxidation. The selective oxidation of the 3′ ribose of RNA by periodate yields a dialdehyde which can then be coupled with an amine or hydrazide reagent (Churchich, Biochim. Biophys. Acta 75:274-276, 1963; Hileman et al. Bioconjug Chem. 5:436-444, 1994).

Individual nucleotides can be derivatized with fluorescent moieties on the base or sugar components. Modification to the base can occur at exocyclic amines or at the carbons of the ring. See, for example, Levina et al., Bioconjug Chem. 4:319-325 (1993). Modification of the sugar moiety can take place at the oxygens of the hydroxyl groups or the carbon atoms of the ribose ring. See, for example, Augustyns et al., Nucleic Acids Symp. Ser. 24;224 (1991); Yamana et al., Bioconjug Chem. 7:715-720 (1996); Guzaev et al., Bioconjug. Chem. 5:501-503 (1994); and Ono et al., Bioconjug. Chem. 4:499-508 (1993), and references contained within, the disclosure of each of which is incorporated herein by reference.

The modified labeled nucleic acids can also be 2′-deoxyribonucleic acids which are labeled at the 3′-hydroxyl via, for example, alkylation or acylation. These labeled nucleic acids will function like dideoxynucleic acids, terminating synthesis, when used in the Sanger method.

Fluorescent G derivatives have also been prepared from the natural base upon its reaction with variously substituted malondialdehydes. See, Leonard and Tolman, in “Chemistry, Biology and Clinical Uses of Nucleoside Analogs,” A. Bloch, ed., Ann. N.Y Acad. Sci. 255:43-58 (1975).

In addition to the various methods for converting the bases of an intact oligonucleotide into their fluorescent analogs, there are a number of methods for introducing fluorescence into an oligonucleotide during its de novo synthesis.

Generally, at least three methods are available for fluorescently tagging a synthetic oligonucleotide. These methods utilize fluorescently tagged supports, fluorescently tagged 5′ modification reagents and fluorescently tagged monomers.

The first of these methods utilizes a fluorescently tagged linker that tethers the oligonucleotide strand to the solid support. When the oligonucleotide strand is cleaved from the solid support, the fluorescent tether remains attached to the oligonucleotide. This method affords an oligonucleotide that is fluorescently labeled at its 3′-end. In a variation on this method, the 3′-end of the nucleic acid is labeled with a linker that bears an amine, or other reactive or masked reactive group, which can be coupled to a reactive fluorophore following cleavage of the oligonucleotide from the solid support. This method is particularly useful when the fluorophore is not stable to the cleavage or deprotection conditions.

A second method relies on the selective labeling of the 5′ terminus of the oligonucleotide chain. Although many methods are known for labeling the 5′ terminus, the most versatile methods make use of phosphoramidites which are derivatized with fluorophore or, if the fluorophore is unstable under the cleaving and deprotection conditions, a protected reactive functional group. The reactive functional group is labeled with a fluorophore following cleavage and deprotection of the oligonucleotide and deprotection of the reactive functional group. The 5′ derivatizing amidites are coupled to the growing nucleic acid strand as a last synthetic cycle that is generally accomplished in the same manner as the previous steps that incorporated single nucleotides.

Many reagents for effecting these conversions are commercially available from chemical houses such as Glen Research (Sterling, Va.). Other agents can be prepared de novo and the commercial agents can be modified by methods well known in the art.

It is also known in the art to prepare oligonucleotides with terminal amino groups that can be used for conjugation of fluorophores and other moieties. See, by way of non-limiting example, U.S. Pat. Nos. 5,118,802 (DNA-reporter conjugates linked via the 2′ or 5′-primary amino group of the 5′-terminal nucleoside) and U.S. Pat. No. 5,118,800 (Oligonucleotides possessing a primary amino group in the terminal nucleotide), both to Smith et al.

C. Fluorescent Peptides, Polypeptides and Proteins

Fluorescent moieties useful in practicing the present invention can be attached to any location on a peptide or protein, including sites on the N-terminus, the C-terminus, a side group, an internal position and combinations thereof.

By way of non-limiting example, a highly fluorescent molecule can be chemically linked to a native amino acid group. The chemical modification occurs on the amino acid side-chain, leaving the carboxyl and amino functionalities free to participate in a polypeptide bond formation. Highly fluorescent dansyl chloride can be linked to the nucleophilic side chains of a variety of amino acids including lysine, arginine, tyrosine, cysteine, histidine, etc., mainly as a sulfonamide for amino groups or sulfate bonds to yield fluorescent derivatives. Such derivatization leaves the ability to form peptide bond intact, allowing for the incorporation of dansyllysine into a protein.

More specifically, non-limiting examples of fluorescent moieties and molecules useful in practicing the present invention include amine-reactive fluorophores, which can react with the N-terminus of a peptide or a side group of an amino acid residue. These include without limitation fluorophores associated with succinimidyl esters and carboxylic acids thereof; aldehydes; sulfonyl chlorides, e.g., dansyl, pyrene, Lissamine rhodamine B and Texas Red derivatives; and arylating reagents (e.g., NBD chloride, NBD fluoride and dichlorotriazines).

Fluorescamine is intrinsically nonfluorescent but reacts rapidly with primary aliphatic amines, including those in peptides and proteins, to yield a blue-green-fluorescent derivative.

The aromatic dialdehydes o-phthaldialdehyde (OPA) and naphthalene-2,3-dicarboxaldehyde (NDA) are essentially nonfluorescent until reacted with a primary amine to yield a fluorescent isoindole.

Sulfonyl chlorides, including dansyl chloride, 1-pyrenesulfonyl chloride and dapoxyl sulfonyl chloride, react with amines to yield blue- or blue-green-fluorescent sulfonamides.

FITC and benzofuran isothiocyanates can be used. A unique method for specific derivatization of the N-terminus of peptides by FITC has been described (“Attachment of a single fluorescent label to peptides for determination by capillary zone electrophoresis.” Zhao J Y, Waldron K C, Miller J, Zhang J Z, Harke H, Dovichi N J. J Chromatogr 608, 239-242, 1992).

N-methylisatoic anhydride and the succinimidyl ester of N-methylanthranilic acid can be used to prepare esters or amides of the small N-methylanthranilic acid fluorophore. The small size of this fluorophore should reduce the likelihood that the label will interfere with the function of the protein.

The type of fluorophore, the site of its attachment to the peptide, the type of linker used to attach the fluorophore and the site of attachment of the peptide to the fluorophore can affect the efficiency of cellular delivery and/or light-induced release of components from the complex. Specifically for fluorescein and fluorescein derivatives having the ring structure of fluorescein, attachment of the peptide at the 5 ring position of the fluorensein fluorophore is preferred.

Fluorophores can be linked to the peptide through linking groups which comprise a spacer portion and groups that form the covalent bonds to the peptide and the fluorophore For fluorescein and fluorescein derivatives having the ring structure of fluorescein, carboxy amine linkers are preferred. Various reagents are commerically available for linking fluorophores to peptides and for generating spacers in the linker. Spacers may include, for example, hydrocarbon spacers (—CH₂),), ether or polyether spacers.

D. Non-Covalent Association of Fluorophores with Nucleic Acids and Proteins

In one embodiment, the fluorophore is non-covalently bound to the translocating peptides and/or nucleic acids of the complexes. Without wishing to be limited to any particular theory, the association of a translocating peptide and a nucleic acid is believed to be non-covalent. When the fluorophore is also non-covalently bound, to the peptide, nucleic acid, or both, the resulting complex is referred to as a fully non-covalent complex.

Nucleic acids that bind fluorophores, including by way of non-limiting example aptamers, can be prepared and used to prepare fully non-covalent complexes of nucleic acids, proteins and fluorophores. Similarly, proteins and peptides that bind fluorophores can be prepared, including without limitation antibodies and derivatives thereof (e.g., single-chain antibodies, camelid antibodies, CDRs, etc.).

A non-covalent specific binding pair can be used to prepare fully non-covalent complexes. In this embodiment, one member of the specific binding pair is associated with the nucleic acid or peptide, and the other member is associated with the fluorophore. The specific binding of members of the pair to each other results in a non-covalent linkage between the nucleic acid or peptide that comprises a member of the binding pair and the fluorophore. For example, biotin and streptavidin can be used to cause the non-covalent association of as fluorophore with a nucleic acid or protein. A strong non-covalent bond is formed between the biotin and avidin moieties (the dissociation constant is approximately 10¹⁵).

In one mode, a biotin moiety can be attached to the fluorophore, and the peptide or oligonucleotide may comprise a streptavidin or avidin moiety. See Sano T, Vajda S, Cantor C R. Genetic engineering of streptavidin, a versatile affinity tag. J Chromatogr B Biomed Sci Appl. 715:85-91, 1998. For example, a fusion protein comprising VP22 translocating protein and strepavidin may be generated and complexed with a biotinylated fluorophore.

VII. Compositions and Methods of Use

Thus, the invention provides conjugates or complexes comprising one or more proteins or peptides, one or more nucleic acid molecules, and optionally one or more fluorophores, produced by the methods of this invention and other methods known to those in the art, including automated and semi-automated methods. For example, an automated device for forming complexes of nucleic acids and poly-Lys is described in U.S. Pat. No. 6,281,005 to Casal, et al. In related aspects, the invention also provides compositions comprising one or more such conjugates or complexes. Compositions according to this aspect of the invention will comprise one or more (e.g., one, two, three, four, five, ten, etc.) of the above-described conjugates or complexes of the invention. In certain such aspects, the compositions may comprise one or more additional components, such as one or more buffer salts, one or more chaotropic agents, one or more detergents, one or more proteins (e.g., one or more enzymes), one or more polymers and the like. The compositions of this aspect of the invention may be in any form, including solid (e.g., dry powder) or solution (particularly in the form of a physiologically compatible buffered salt solution comprising one or more of the conjugates of the invention).

A. Pharmaceutical Compositions

Certain compositions of the invention are particularly formulated for use as pharmaceutical compositions for use in prophylactic, diagnostic or therapeutic applications. Such compositions will typically comprise one or more of the conjugates, complexes or compositions of the invention and one or more pharmaceutically acceptable carriers or excipients. The term “pharmaceutically acceptable carrier or excipient,” as used herein, refers to a non-toxic solid, semisolid or liquid filler, diluent, encapsulating material or formulation auxiliary of any type that is capable of being tolerated by a recipient animal, including a human or other mammal, into which the pharmaceutical composition is introduced, without adverse effects resulting from its addition.

The pharmaceutical compositions of the invention may be administered to a recipient via any suitable mode of administration, such as orally, rectally, parenterally, intrasystemically, vaginally, intraperitoneally, topically (as by powders, ointments, drops or transdermal patch), buccally, as an oral or nasal spray or by inhalation. The term “parenteral” as used herein refers to modes of administration that include intravenous, intramuscular, intraperitoneal, intracisternal, subcutaneous and intra-articular injection and infusion.

Pharmaceutical compositions provided by the present invention for parenteral injection can comprise pharmaceutically acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, poly(ethylene glycol), and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

Such pharmaceutical compositions of the present invention may also contain adjuvants such as preservatives, wetting agents, emulsifying agents and dispersing agents. Prevention of the action of microorganisms may be ensured by the inclusion of various antibacterial and antifungal agents, for example, paraben, benzyl alcohol, chlorobutanol, phenol, sorbic acid, and the like. It may also be desirable to include osmotic agents such as sugars, sodium chloride and the like. Prolonged absorption of the injectable pharmaceutical form may be brought about by the inclusion of agents that delay absorption, such as aluminum monostearate, hydrogels and gelatin.

In some cases, in order to prolong the effect of the drugs, it is desirable to slow the absorption from subcutaneous or intramuscular injection. This may be accomplished by the use of a liquid suspension of crystalline or amorphous material with poor solubility in aqueous body fluids. The rate of absorption of the drug then depends upon its rate of dissolution, which, in turn, may depend upon its physical form. Alternatively, delayed absorption of a parenterally administered drug form is accomplished by dissolving or suspending the drug in an oil vehicle.

Injectable depot forms are made by forming microencapsulated matrices of the drug in biodegradable polymers such as polylactide-polyglycolide. Depending upon the ratio of drug to carrier polymer and the nature of the particular carrier polymer employed, the rate of drug release can be controlled. Examples of other biodegradable polymers include biocompatible poly(orthoesters) and poly(anhydrides). Depot injectable formulations are also prepared by entrapping the drug in liposomes or microemulsions that are compatible with body tissues.

The injectable formulations can be sterilized, for example, by filtration through a bacteria-retaining filter, or by incorporating sterilizing agents in the form of sterile solid compositions that can be dissolved or dispersed in sterile water or other sterile injectable medium prior to use.

Solid dosage forms for oral administration include capsules, tablets, pills, powders and granules. In such solid dosage forms, the active compounds are mixed with at least one pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and gum acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) accelerators of absorption, such as quaternary ammonium compounds, g) wetting agents such as, for example, cetyl alcohol and glycerol monostearate, h) adsorbents such as kaolin and bentonite clay, and i) lubricants such as talc, calcium stearate, magnesium stearate, solid poly(ethylene glycols), sodium lauryl sulfate, and mixtures thereof. In the case of capsules, tablets and pills, the dosage form may also comprise buffering agents.

Solid compositions of a similar type may also be employed as fillers in soft- and hard-filled gelatin capsules using such excipients as lactose (milk sugar) as well as high molecular weight poly(ethylene glycols) and the like.

The solid dosage forms of tablets, dragees, capsules, pills and granules can be prepared with coatings and shells such as enteric or chronomodulating coatings and other coatings well known in the pharmaceutical formulating art. They may optionally contain opacifying agents and can also be of such a composition that they release the active ingredient(s) only, or preferentially, in a certain part of the gastrointestinal tract, optionally, in a delayed manner. Examples of embedding compositions that can be used include polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, poly(ethylene glycols) and fatty acid esters of sorbitan, and mixtures thereof.

In addition to inert diluents, the oral compositions can also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Topical administration includes administration to the skin or mucosa, including surfaces of the lung and eye. Compositions for topical administration, including those for inhalation, may be prepared as a dry powder which may be pressurized or non-pressurized. In non-pressurized powder compositions, the active ingredients in finely divided form may be used in admixture with a larger-sized pharmaceutically acceptable inert carrier comprising particles having a size, for example, of up to 100 micrometers in diameter. Suitable inert carriers include sugars such as lactose and sucrose. Desirably, at least 95% by weight of the particles of the active ingredient have an effective particle size in the range of 0.01 to 10 micrometer.

Alternatively, the pharmaceutical composition may be pressurized and contain a compressed gas, such as nitrogen or a liquefied gas propellant. The liquefied propellant medium and indeed the total composition may be preferably such that the active ingredients do not dissolve therein to any substantial extent. The pressurized composition may also contain a surface-active agent. The surface-active agent may be a liquid or solid non-ionic surface-active agent or may be a solid anionic surface-active agent. It is preferable to use the solid anionic surface-active agent in the form of a sodium salt.

A further form of topical administration is to the eye. In this mode of administration, the conjugates or compositions of the invention are delivered in a pharmaceutically acceptable ophthalmic vehicle, such that the active compounds are maintained in contact with the ocular surface for a sufficient time period to allow the compounds to penetrate the conjunctiva or the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material.

Compositions for rectal or vaginal administration are preferably suppositories that can be prepared by mixing the conjugates or compositions of the invention with suitable non-irritating excipients or carriers such as cocoa butter, PEG or a suppository wax, which are solid at room temperature but liquid at body temperature and therefore melt in the rectum or vaginal cavity and release the drugs.

The pharmaceutical compositions used in the present therapeutic methods may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. In addition to one or more of the conjugates or compositions of the invention, the present pharmaceutical compositions in liposome form can also contain one or more stabilizers, preservatives, excipients, and the like. The preferred lipids are the phospholipids and the phosphatidyl cholines (lecithins), both natural and synthetic. Methods to form liposomes are known in the art (see, e.g., Zalipsky, S., et al., U.S. Pat. No. 5,395,619). Liposomes that comprise phospholipids that are conjugated to poly(ethylene glycol) (“PEG”), most commonly phosphatidyl ethanolamine coupled to monomethoxy-PEG, have advantageous properties, including prolonged lifetimes in the blood circulation of mammals (Fisher, D., U.S. Pat. No. 6,132,763).

B. Uses

As noted elsewhere herein, the conjugates and compositions of the present invention are advantageously used in methods for delivering one or more components (e.g., one or more peptides and/or one or more nucleic acid molecules and/or one or more fluorophores) of the conjugates and compositions to cells, tissues, organs or organisms. In particular, the invention provides controlled delivery of the one or more components of the complexes or compositions to cells, tissues, organs or organisms, thereby providing the user with the ability to regulate, temporally and spacially, the amount of a particular component that is released for activity on the cells, tissues, organs or organisms.

In general, such methods of the invention involve one or more activities. For example, one such method of the invention comprises: (a) preparing one or more complexes or compositions of the invention as detailed herein; (b) contacting one or more cells, tissues, organs or organisms with the one or more complexes or compositions, under conditions favoring the uptake of the one or more complexes or compositions of the invention by the cells, tissues, organs or organisms; and (c) treating the cells, tissues, organs or organisms that contain the one or more complexes or compositions of the invention with a treatment that releases one or more of the bioactive components of the conjugates or compositions into the cells, tissues, organs or organisms. In certain embodiments, for example, the releasing treatment comprises irradiating the cells, tissues, organs or organisms with electromagnetic radiation, particularly light, at a wavelength and intensity and for a duration of time sufficient to activate one or more radiative-sensitive components (e.g., one or more fluorophores) of the complexes or compositions, thereby releasing one or more of the bioactive components (e.g., one or more peptides and/or one or more nucleic acids) into the cells, tissues, organs or organisms. In certain such aspects of the invention, the treatment comprises irradiating the cells, tissues, organs or organisms with light having an excitation wavelength falling within the range of wavelengths of from about 200 nm to about 800 nm. Other wavelengths suitable for use in accordance with the methods of the invention are detailed hereinabove, and will be familiar to the ordinarily skilled artisan.

Once the bioactive components of the complexes and/or compositions of the invention have been released into the cells, tissues, organs or organisms, the components proceed to carry out their intended biological functions. For example, peptide components released into the cells, tissues, organs or organisms may proceed to bind to receptors or other compounds or components within the cells, tissues, organs or organisms; to participate in metabolic reactions within the cells, tissues, organs or organisms; to carry out, upregulate or activate, or downregulate or inhibit, one or more enzymatic activities within the cells, tissues, organs or organisms; to provide a missing structural component to the cells, tissues, organs or organisms; to provide one or more nutritional needs to the cells, tissues, organs or organisms; to inhibit, treat, reverse or otherwise ameliorate one or more processes or symptoms of a disease or physical disorder; and the like. In other examples, nucleic acid components released inato the cells, tissues, organs or organisms may proceed to bind to receptors or other compounds or components within the cells, tissues, organs or organisms; to become incorporated into the genetic material within the cells, tissues, organs or organisms, whether chromosomal or extrachromosomal, genomic or otherwise; to carry out, upregulate or activate, or downregulate or inhibit, one or more enzymatic activities within the cells, tissues, organs or organisms; to provide a missing genetic component to the cells, tissues, organs or organisms; to increase or decrease the copy number of one or more genes within the cells, tissues, organs or organisms; to inhibit, treat, reverse or otherwise ameliorate one or more processes or symptoms of a disease or physical disorder; and the like. In related aspects, the complexes and compositions of the invention can be used to produce transgenic cells, tissues, organs or organisms, including non-human transgenic animals such as mice, rats, dogs, cows, pigs, rabbits, dogs, monkeys and the like, using methods (such as nuclear transfer cloning) that are well-known in the art and that will be familiar to the ordinarily skilled artisan (see, e.g., U.S. Pat. Nos. 5,322,775, 5,366,894, 5,476,995, 5,650,503 and 5,861,299; WIPO/PCT publication nos. WO 98/37183 and WO 00/42174; U.S. patent application publication no. 0012660-A1 (published on Jan. 31, 2002); Dai et al., Nature Biotechnology 20: 251-255 (2002); Betthauser et al., Nature Biotechnology 18: 1055-1059 (2000); Onishi et al., Science 289:1188-1190 (2000); and Polejaeva et al., Nature 407:86-90 (2000). The disclosures of all of these documents are incorporated herein by reference in their entireties).

C. Dose Regimens

The conjugates, complexes or compositions of the invention can be administered in vitro, ex vivo or in vivo to cells, tissues, organs or organisms to deliver one or more bioactive components (i.e., one or more peptides or nucleic acid molecules) thereto. One of ordinary skill will appreciate that effective amounts of a given active compound, conjugate, complex or composition can be determined empirically and may be employed in pure form or, where such forms exist, in pharmaceutically acceptable formulation or prodrug form. The compounds, conjugates, complexes or compositions of the invention may be administered to an animal (including a mammal, such as a human) patient in need thereof as veterinary or pharmaceutical compositions in combination with one or more pharmaceutically acceptable excipients. It will be understood that, when administered to a human patient, the total daily, weekly or monthly usage of the compounds and compositions of the present invention will be decided by the attending physician within the scope of sound medical judgment. The therapeutically effective dose level for any particular patient will depend upon a variety of factors including the type and degree of the cellular response to be achieved; the identity and/or activity of the specific compound(s), conjugate(s), complex(es) or composition(s) employed; the age, body weight or surface area, general health, gender and diet of the patient; the time of administration, route of administration, and rate of excretion of the active compound(s); the duration of the treatment; other drugs used in combination or coincidental with the specific compound(s), conjugate(s), complex(es) or composition(s); and like factors that are well known to those of ordinary skill in the pharmaceutical and medical arts. For example, it is well within the skill of the art to start doses of a given compound, conjugate, complex or composition of the invention at levels lower than those required to achieve the desired therapeutic effect and to gradually increase the dosages until the desired effect is achieved.

Dose regimens may also be arranged in a patient-specific manner to provide a predetermined concentration of a given active compound in the blood, as determined by techniques accepted and routine in the art, e.g. size-exclusion, ion-exchange or reversed-phase BPLC. Thus, patient dose regimens may be adjusted to achieve relatively constant blood levels, as measured by HPLC, according to methods that are routine and familiar to those of ordinary skill in the medical, pharmaceutical and/or pharmacological arts.

D. Diagnostic and Therapeutic Uses

A diagnostic use of a conjugate of the invention might be for locating an antigenic moiety, e.g., a cancer, within the body of an animal, especially a human, by administration of a complex or composition of the invention, in which the complex or conjugate is labeled or comprises one or more detectable labels so as to enable detection, e.g., by optical, radiometric, fluorescent or resonant detection according to art-known methods. Hence, in another aspect of the invention, the conjugates and compositions of the invention may be used in diagnostic or therapeutic methods, for example in diagnosing, treating or preventing a variety of physical disorders in an animal, particularly a mammal such as a human, predisposed to or suffering from such a disorder. In such approaches, the goal of the therapy is to delay or prevent the development of the disorder, and/or to cure or induce a remission of the disorder, and/or to decrease or minimize the side effects of other therapeutic regimens. Hence, the complexes and compositions of the present invention may be used for protection, suppression or treatment of physical disorders, such as infections or diseases. The term “protection” from a physical disorder, as used herein, encompasses “prevention,” “suppression” and “treatment.” “Prevention” involves the administration of a complex or composition of the invention prior to the induction of the disease or physical disorder, while “suppression” involves the administration of the complex or composition prior to the clinical appearance of the disease; hence, “prevention” and “suppression” of a physical disorder typically are undertaken in an animal that is predisposed to or susceptible to the disorder, but that is not yet suffering therefrom. “Treatment” of a physical disorder, however, involves administration of the therapeutic complex or composition of the invention after the appearance of the disease. It will be understood that in human and veterinary medicine, it is not always possible to distinguish between “preventing” and “suppressing” a physical disorder. In many cases, the ultimate inductive event or events may be unknown or latent, and neither the patient nor the physician may be aware of the inductive event until well after its occurrence. Therefore, it is common to use the term “prophylaxis,” as distinct from “treatment,” to encompass both “preventing” and “suppressing” as defined herein. The term “protection,” used in accordance with the methods of the present invention, therefore is meant to include “prophylaxis.”

Methods according to this aspect of the invention may comprise one or more steps that allow the clinician to achieve the above-described therapeutic goals. One such method of the invention may comprise, for example:

(a) identifying an animal (preferably a mammal, such as a human) suffering from or predisposed to a physical disorder; and

(b) administering to the animal an effective amount of one or more of the conjugates, complexes or compositions of the present invention as described herein, particularly one or more complexes comprising one or more peptides, one or more nucleic acids, and one or more fluorophores (or one or more pharmaceutical compositions comprising such conjugates), such that the administration of the conjugate, complex or composition prevents, delays or diagnoses the development of, or cures or induces remission of, the physical disorder in the animal.

As used herein, an animal that is “predisposed to” a physical disorder is defined as an animal that does not exhibit a plurality of overt physical symptoms of the disorder but that is genetically, physiologically or otherwise at risk for developing the disorder. In the present methods, the identification of an animal (such as a mammal, including a human) that is predisposed to, at risk for, or suffering from a given physical disorder may be accomplished according to standard art-known methods that will be familiar to the ordinarily skilled clinician, including, for example, radiological assays, biochemical assays (e.g., assays of the relative levels of particular peptides, proteins, electrolytes, etc., in a sample obtained from an animal), surgical methods, genetic screening, family history, physical palpation, pathological or histological tests (e.g., microscopic evaluation of tissue or bodily fluid samples or smears, immunological assays, etc.), testing of bodily fluids (e.g., blood, serum, plasma, cerebrospinal fluid, urine, saliva, semen and the like), imaging, (e.g., radiologic, fluorescent, optical, resonant (e.g., using nuclear magnetic resonance (NMR) or electron spin resonance (ESR)), etc. Once an animal has been identified by one or more such methods, the animal may be aggressively and/or proactively treated to prevent, suppress, delay or cure the physical disorder.

Physical disorders that can be prevented, diagnosed or treated with the complexes, compositions and methods of the present invention include any physical disorders for which the peptide and/or nucleic acid component(s) of the complexes or compositions may be used in the prevention, diagnosis or treatment. Such disorders include, but are not limited to, a variety of cancers (e.g., breast cancers, uterine cancers, ovarian cancers, prostate cancers, testicular cancers, leukemias, lymphomas, lung cancers, neurological cancers, skin cancers, head and neck cancers, bone cancers, colon and other gastrointestinal cancers, pancreatic cancers, bladder cancers, kidney cancers and other carcinomas, sarcomas, adenomas and myelomas); infectious diseases (e.g., bacterial diseases, fungal diseases, viral diseases (including hepatitis and HIV/AIDS), parasitic diseases, and the like); genetic disorders (e.g., cystic fibrosis, amyotrophic lateral sclerosis, muscular dystrophy, Gaucher's disease, Pompe's disease, severe combined immunodeficiency disorder and the like), anemia, neutropenia, hemophilia and other blood disorders; neurological disorders (e.g., multiple sclerosis and Alzheimer's disease); enzymatic disorders (e.g., gout, uremia, hypercholesterolemia, and the like); disorders of uncertain or multifocal etiology (e.g., cardiovascular disease, hypertension, and the like); and other disorders of medical importance that will be readily familiar to the ordinarily skilled artisan. The complexes, compositions and methods of the present invention may also be used in the prevention of disease progression, such as in chemoprevention of the progression of a premalignant lesion to a malignant lesion.

The therapeutic methods of the invention thus use one or more conjugates, complexes or compositions of the invention, or one or more of the pharmaceutical compositions of the invention, that may be administered to an animal in need thereof by a variety of routes of administration, including orally, rectally, parenterally (including intravenously, intramuscularly, intraperitoneally, intracisternally, subcutaneously and intra-articular injection and infusion), intrasystemically, vaginally, intraperitoneally, topically (as by powders, ointments, drops or transdermal patch), buccally, as an oral or nasal spray or by inhalation. By the invention, an effective amount of the conjugates, complexes or compositions can be administered in vitro, ex vivo or in vivo to cells or to animals suffering from or predisposed to a particular disorder, thereby preventing, delaying, diagnosing or treating the disorder in the animal. As used herein, “an effective amount of a conjugate (or complex or composition)” refers to an amount such that the conjugate (or complex or composition) carries out the biological activity of the bioactive component (i.e., the peptide and/or nucleic acid component) of the conjugate/complex/composition, thereby preventing, delaying, diagnosing, treating or curing the physical disorder in the animal to which the conjugate, complex or composition of the invention has been administered. One of ordinary skill will appreciate that effective amounts of the conjugates, complexes or compositions of the invention can be determined empirically, according to standard methods well-known to those of ordinary skill in the pharmaceutical and medical arts; see, e.g., Beers, M. H., et al., eds. (1999) Merck Manual of Diagnosis & Therapy, 17th edition, Merck and Co., Rahway, N J; Hardman, J. G., et al., eds. (2001) Goodman and Gilman's The Pharmacological Basis of Therapeutics, 10th edition, McGraw-Hill Professional Publishing, Elmsford, N.Y.; Speight, T. M., et al., eds. (1997) Avery's Drug Treatment: Principles and Practice of Clinical Pharmacology and Therapeutics, 4th edition, Blackwell Science, Inc., Boston; Katzung, B. G. (2000) Basic and Clinical Phamacology, 8th edition, Appleton and Lange, Norwalk, Conn.; which references and references cited therein are incorporated entirely herein by reference.

It will be understood that, when administered to a human patient, the total daily, weekly or monthly dosage of the conjugates, complexes and compositions of the present invention will be decided by the attending physician within the scope of sound medical judgment. For example, satisfactory results are obtained by administration of certain of the conjugates, complexes or compositions of the invention at appropriate dosages depending on the specific bioactive compound used, which dosages will be readily familiar to the ordinarily skilled artisan or which may be readily determined empirically using only routine experimentation. According to this aspect of the invention, the conjugates, complexes or compositions can be administered once or, in divided doses, e.g., twice per day or per week or per month. Appropriate dose regimens for various modes of administration (e.g., parenteral, subcutaneous, intramuscular, intraocular, intranasal, etc.) can also be readily determined empirically, using only routine experimentation, or will be readily apparent to the ordinarily skilled artisan, depending on the identity of the bioactive component (i.e., the peptide and/or nucleic acid component) of the conjugate, complex or composition.

In additional applications, the conjugates, complexes and compositions of the invention may be used to specifically target a diagnostic or therapeutic agent to a cell, tissue, organ or organism that expresses a receptor for, binds, incorporates or otherwise can take up, the bioactive component (i.e., the peptide and/or nucleic acid component) of the conjugate, complex or composition. Methods according to this aspect of the invention may comprise, for example, contacting the cell, tissue, organ or organism with one or more conjugates, complexes or compositions of the invention, which additionally comprise one or more diagnostic or therapeutic agents, such that the conjugate, complex or composition is taken up by the cell, tissue, organ or organism by any mechanism (e.g., by receptor-mediated endocytosis, pinocytosis, phagocytosis, diffusion, etc.), thereby delivering the diagnostic or therapeutic agent to the cell, tissue, organ or organism. The diagnostic or therapeutic agent used in accordance with this aspect of the invention may be, but is not limited to, at least one agent selected from a nucleic acid, an organic compound, a protein or peptide, an antibody, an enzyme, a glycoprotein, a lipoprotein, an element, a lipid, a saccharide, an isotope, a carbohydrate, an imaging agent, a detectable probe, or any combination thereof, which may be detectably labeled as described herein. A therapeutic agent used in this aspect of the present invention may have a therapeutic effect on the target cell (or tissue, organ or organism), the effect being selected from, but not limited to, correcting a defective gene or protein, a drug action, a toxic effect, a growth stimulating effect, a growth inhibiting effect, a metabolic effect, a catabolic affect, an anabolic effect, an antiviral effect, an antifungal effect, an antibacterial effect, a hormonal effect, a neurohumoral effect, a cell differentiation stimulatory effect, a cell differentiation inhibitory effect, a neuromodulatory effect, an anti-neoplastic effect, an anti-tumor effect, an insulin stimulating or inhibiting effect, a bone marrow stimulating effect, a pluripotent stem cell stimulating effect, an immune system stimulating effect, and any other known therapeutic effect that may be provided by a therapeutic agent delivered to a cell (or tissue, organ or organism) via a delivery system according to this aspect of the present invention.

Such additional therapeutic agents may be selected from, but are not limited to, known and new compounds and compositions including antibiotics, steroids, cytotoxic agents, vasoactive drugs, antibodies and other therapeutic agents. Non-limiting examples of such agents include antibiotics and other drugs used in the treatment of bacterial shock, such as gentamycin, tobramycin, nafcillin, parenteral cephalosporins, etc.; adrenal corticosteroids and analogs thereof, such as dexamethasone, mitigate the cellular injury caused by endotoxins; vasoactive drugs, such as an alpha adrenergic receptor blocking agent (e.g., phenoxybenzamine), a beta adrenergic receptor agonist (e.g., isoproterenol), and dopamine.

The conjugates, complexes and compositions of the invention may also be used for diagnosis of disease and to monitor therapeutic response. In certain such methods, the conjugates, complexes or compositions of the invention may comprise one or more detectable labels (such as those described elsewhere herein). In specific such methods, these detectably labeled conjugates, complexes or compositions of the invention may be used to detect cells, tissues, organs or organisms expressing receptors for, or otherwise taking up, the bioactive component (i.e., the peptide and/or nucleic acid component) of the conjugates, complexes or compositions. In one example of such a method, the cell, tissue, organ or organism is contacted with one or more of the conjugates, complexes or compositions of the invention under conditions that favor the uptake of the conjugate by the cell, tissue or organism (e.g., by binding of the conjugate to a cell-surface receptor or by pinocytosis or diffusion of the conjugate into the cell), and then detecting the conjugate bound to or incorporated into the cell using detection means specific to the label used (e.g., fluorescence detection for fluorescently labeled conjugates; magnetic resonance imaging for magnetically labeled conjugates; radioimaging for radiolabeled conjugates; etc.). Other uses of such detectably labeled conjugates may include, for example, imaging a cell, tissue, organ or organism, or the internal structure of an animal (including a human), by administering an effective amount of a labeled form of one or more of the conjugates of the invention and measuring detectable radiation associated with the cell, tissue, organ or organism (or animal). Methods of detecting various types of labels and their uses in diagnostic and therapeutic imaging are well known to the ordinarily skilled artisan, and are described elsewhere herein.

In another aspect, the conjugates and compositions of the invention may be used in methods to modulate the concentration or activity of a specific receptor for the bioactive component of the conjugate on the surface of a cell that expresses such a receptor. By “modulating” the activity of a given receptor is meant that the conjugate, upon binding to the receptor, either activates or inhibits the physiological activity (e.g., the intracellular signaling cascade) mediated through that receptor. While not intending to be bound by any particular mechanistic explanation for the regulatory activity of the conjugates of the present invention, such conjugates can antagonize the physiological activity of a cellular receptor by binding to the receptor via the bioactive component of the conjugate, thereby blocking the binding of the natural agonist (e.g., the unconjugated bioactive component) and preventing activation of the receptor by the natural agonist, while not inducing a substantial activation of the physiological activity of the receptor itself. Methods according to this aspect of the invention may comprise one or more steps, for example contacting the cell (which may be done in vitro or in vivo) with one or more of the conjugates of the invention, under conditions such that the conjugate (i.e., the bioactive component portion of the conjugate) binds to a receptor for the bioactive component on the cell surface but does not substantially activate the receptor. Such methods will be useful in a variety of diagnostic, and therapeutic applications, as the ordinarily skilled artisan will readily appreciate.

VIII. Kits

The invention also provides kits comprising the conjugates and/or compositions of the invention. Such kits typically comprise a carrier, such as a box, carton, tube or the like, having in close confinement therein one or more containers, such as vials, tubes, ampules, bottles and the like, wherein a first container contains one or more of the conjugates and/or compositions of the present invention. The kits encompassed by this aspect of the present invention may further comprise one or more additional components (e.g., reagents and compounds) necessary for carrying out one or more particular applications of the conjugates and compositions of the present invention, such as one or more components useful for the diagnosis, treatment or prevention of a particular disease or physical disorder (e.g., one or more additional therapeutic compounds or compositions, one or more diagnostic reagents, one or more carriers or excipients, and the like), one or more additional conjugates or compositions of the invention, one or more sets of instructions, and the like.

It will be readily apparent to one of ordinary skill in the relevant arts that other suitable modifications and adaptations to the methods and applications described herein may be made without departing from the scope of the invention or any embodiment thereof. Having now described the present invention in detail, the same will be more clearly understood by reference to the following examples, which are included herewith for purposes of illustration only and are not intended to be limiting of the invention.

### THE EXAMPLES

### Example 1

### Cellular Delivery Polypeptides

Some of the peptides used in the Examples are shown in Table 5. Synthetic polypeptides were prepared by standard methods known in the art. Standard cloning techniques were used to prepare fusion proteins. The expression vector pCR®T7/VP22-1 (Invitrogen) was used.

TABLE 5
Peptide Sequences

| | | | |
|---|---|---|---|
| Designation | Amino Acid Sequence | pI ⁽¹⁾ | SEQ ID NO.: |

| | | | |
|---|---|---|---|
| R7 | RRRRRRR | 12.78 | 3 |
| R9 | RRRRRRRRR | 10.90 | 4 |
| R11 | RRRRRRRRRRR | 13.00 | 5 |
| RRG | RRGRGRGRR | 12.70 | 6 |
| RRQ | RRQRQRGRR | 12.70 | 7 |
| RG9 | RRRRGRRRR | 12.85 | 8 |
| K7 | KKKKKKK | 10.78 | 9 |
| K9 | KKKKKKKKK | 10.90 | 10 |
| K11 | KKKKKKKKKKK | 11.00 | 11 |
| PTD3 | YARKARRQARR ⁽²⁾ | 12.18 | 12 |
| PTDA | YAAKAAAQAAA ⁽²⁾ | 8.59 | 13 |
| Ant(42-58) | ERQIKIFFQNRRMKFKK ⁽³⁾ | 11.7 | 14 |
| AntFF(42-58) | ERQIKIWFQNRRMKWKK ⁽³⁾ | 11.7 | 15 |
| Ant(43-58) | RQIKIFFQNRRMKFKK ⁽³⁾ | 12.31 | |
| AntFF(43-58) | RQIKIWFQNRRMKWKK ⁽³⁾ | 12.31 | |
| Ant(41-60) | TERQIKIFFQNRRMKFKKKE ⁽³⁾ | 11.12 | |
| VP22(159-301) ⁽⁴⁾ | — | 11.31 | — |
| Poly-D-Lys | (K)ₙ, where n ≈ 35-100 ⁽⁴⁾ | ˜11.5- | — |
| | | 12.0 | |
| Poly-D-Arg | (R)ₙ, where n ≈ 35-100 ⁽⁵⁾ | ˜13.5- | — |
| | | 14.0 | |

⁽¹⁾ pI predicted on-line by Swissprot (http://us.expasy.org/tools/pi_toolref.html), September 2002 version. See Bjellqvist, B., et al., Electrophoresis 14, 1023-1031 (1993); Bjellqvist, B., et al., Electrophoresis 15, 529-539 (1994); and Wilkins, M. R., et al., Protein Identification and Analysis Tools in the ExPASy Server in: 2-D Proteome Analysis Protocols, Editor A. J. Link. Humana Press, New Jersey (1998).
⁽²⁾ See Ho et al., 2001.
⁽³⁾ See Derossi, D., et al., J. Biol. Chem. 269: 10444-10450 (1994).
⁽⁴⁾ See: Elliot and O'Hare (1997) Cell 88: 223-33; Dilber et al. (1999) Gene Ther. 6: 12-21; and Phelan et al. (1998) Nature Biotechnol. 16: 440-3).
⁽⁴⁾ Sigma # P-6403.
⁽⁵⁾ Sigma # P-4663.

### Example 2

Controlled Delivery and Redistribution of Fluorescent Oligonucleotides

2.1. Materials and Methods

The peptide-oligonucleotide complexes were prepared as follows. Each peptide was diluted to a concentration of 1 μM in 10 μl PBS (Invitrogen). A FITC-labeled oligonucleotide (FITC-5′-TCCCGCGCACTTGATGCATT*) (SEQ ID NO:16) was used (Normand, N., et al., J. Biol. Chem.. 276:15042-15050 (2001)). The FITC-labeled oligonucleotide was synthesized according to techniques known in the art (see, generally: Hagmar et al. Synthesis and characterisation of fluorescent oligonucleotides. Effect of internal labelling on protein recognition. Biochim Biophys Acta. 1244:259-268, 1995; Aubert et al. Optimized synthesis of phosphorothioate oligodeoxyribonucleotides substituted with a 5′-protected thiol function and a 3′-amino group. Nucleic Acids Res. 28:818-825, 2000; and Dubey I, Pratviel G, Meunier B. Modification of the thiourea linkage of a fluorescein-oligonucleotide conjugate to a guanidinium motif during ammonia deprotection. Bioconjug Chem 9:627-632, 1998).

The oligonucleotide was diluted to 0.5 μM in 10 μl PBS, which was then combined with the 10 ul peptide solution described above. The peptide-oligonucleotide mix was incubated for 10 min at room temperature.

CHO cells were seeded in 24 well plates at a density of 5×10⁴ cells/well. Twenty-four hours (24 h) later, the medium in each well of cells was replaced with 0.5 ml HAM/10% fetal bovine serum (Invitrogen). The peptide-oligonucleotide mixture was then added to the medium and allowed to incubate with the cells for 16 hr at 37° C.

Next, the medium was replaced with fresh media and cells were observed using a Nikon fluorescence microscope equipped with FITC filter and a 40× objective. Cells were photographed immediately (t=0 time point for all Figures). Cells were observed for 30 s, then the fluorescence light path was closed and cells were allowed to incubate for a further 90 s without illumination. The cells were then photographed a second time (t=2 time point for all Figures).

2.2. Results: Arginine-Rich Peptides

Following the 16 hr incubation of cells with the peptide-oligonucleotide mixture, complexes containing the R9 (RRRRRRRRR) (SEQ ID NO:4) and PTD3 (YARKARRQARR) (SEQ ID NO: 12) peptides were detected in cells by fluorescence microscopy, whereas PTDA (YAAKAAAQAAA) (SEQ ID NO:13) was not. Following illumination for 30 s and a further 90 s incubation without illumination, redistribution of fluorescence could be detected in experiments using the R9 or PTD3 peptides. That is, illumination of cells comprising cytoplasmic fluorescent particles resulted in more-or-less uniform cytoplasmic staining followed by nuclear accumulation.

The time course of fluorescence redistribution observed using the R9 peptide/oligonucleotide complex (FIG. 1) was typical of that seen with each peptide. After 30 s continuous illumination redistribution could be detected in some cells. After 1 min continuous illumination, redistribution in most cells was seen, and by 2 min redistribution was complete. Similar patterns of redistribution were seen with PTD3, whereas PTDA did not appear to form complexes with the FITC oligonucleotide. With the poly-Lys and longer poly-Arg (i.e., Argₙ, where n=˜35-100) peptides, redistribution was not seen although uptake could be detected.

2.3. Results: Ant Peptides

Following 16 hr incubation, complexes containing Ant(42-58) and AntFF(42-58)peptides were detected in cells by fluorescence microscopy. Following illumination for 30 s and a further 90 s incubation without illumination, redistribution of fluorescence could be detected using the Ant(42-58) peptide and corresponding AntFF peptide.

AntFF (42-58) is a mutant form of the Ant(42-58) peptide in which two tryptophan residues are substituted for phenylalanines. Uptake of a FITC-labeled oligonucleotide complexed with AntFF(42-58) was observed in th e present work. In addition, following illumination, redistribution of the fluorescence signal was seen in some cells.

Derossi, D., et al., J. Biol. Chem. 269:10444-10450 (1994) had shown that a 16 amino acid peptide, Ant(43-58), and a 20 amino acid peptide, Ant(41-60), derived from the third helix of the Antennapedia homeodomain translocate through biological membranes. Two other peptides from this region Ant(46-60) and Ant(41-55) were reported not to be internalized in cells. A mutant of Ant(43-58) in which two tryptophan residues are substituted for phenylalanines designated AntFF was reported to exhibit a much lower efficiency for internalization than the wild-type peptide.

The finding of this work suggests, however, that the translocating property associated with the AntFF peptide alone (without oligonucleotide) need not be retained in order for peptide-oligonucleotide complex delivery and release.

### Example 3

### Delivery and Activation of Antisense Oligonucleotides

The R9 peptide (SEQ ID NO:4) was used to make complexes with a FITC-antisense oligonucleotide directed against the human raf kinase. The FITC-anti-raf oligonucleotide, and its delivery to the lung carcinoma cell line A-549 using VP22, has been described (Normond et al., 2001).

Complexes of the FITC-anti-raf oligonucleotide with either the VP22(159-301) protein or the R9 peptide were prepared. The antisense effect was evaluated by contacting A549 cells with complexes comprising the antisense oligonucleotide and either the VP22(159-301) protein or the R9 peptide.

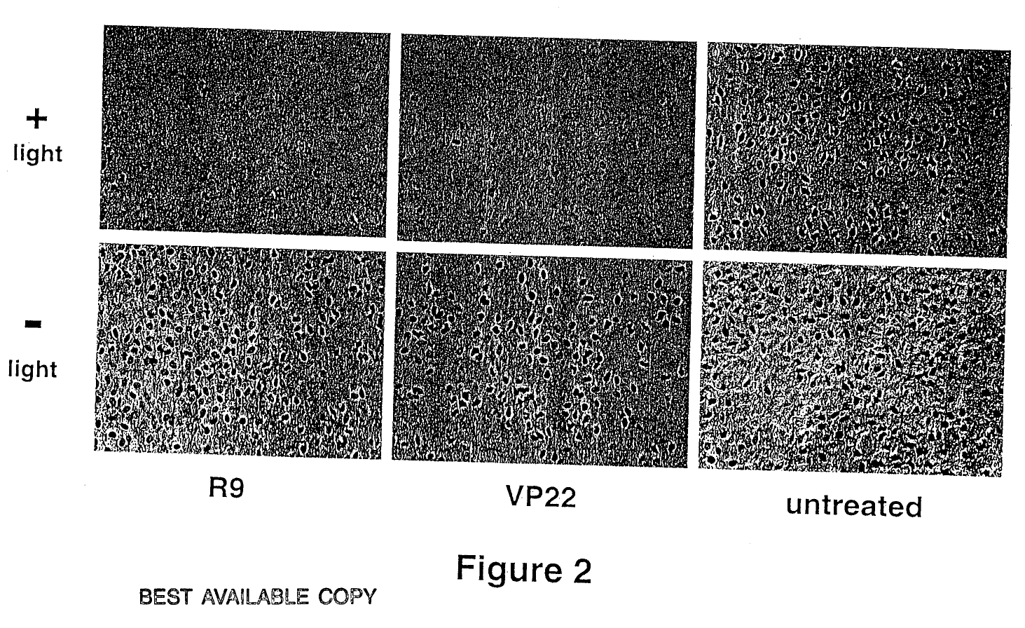

Cells were grown in 24 well plates and were either illuminated by putting the plate on an overhead projector (3M model 9100 with a 360 W bulb) for 5 min, or kept dark. Sixty hours after illumination, cells were labeled for 1 hour with Bromo-deoxyuridine (BrdU). Labeling and immunocytochemical detection were performed using anti-BrdU and alkaline phosphatase conjugated secondary antibodies according to the manufacturers' instructions (Roche). A reduced number of BrdU labeled cells and a reduction in the intensity of BrdU labeling indicates an effect on raf dependent signaling, leading to fewer cells progressing through the cell cycle. The observed effects appeared similar with oligonucleotide delivery using VP22 or R9. This result shows that the R9 peptide can be used for antisense oligonucleotide delivery and that the antisense effect is only seen when cells are illuminated, corresponding with dispersal of the peptide-oligonucleotide complex (FIG. 2).

### Example 4

### Evaluation of Cellular Delivery Polypeptides

The following peptides were tested for delivery and light dependent release of a FITC-labeled antisense oligonucleotide (FITC-anti-raf oligonucleotide) as described above: R7, R9, R11, K7, K9, K11, RG9 (RRRRGRRRR) (SEQ ID NO:8), RRG (RRGRGRGRR) (SEQ ID NO:6), and RRQ (RRQRQRGRR) (SEQ ID NO:7). The amino acid sequences, SEQ ID NOs., and predicted pIs of these peptides are shown above shown in Table 5.

After overnight incubation with peptide-oligonucleotide complexes, cells were illuminated under the fluorescence microscope and observed to check whether or not the initial fluorescence distribution was similar to that seen for the R9-oligonucleotide complexes. FITC-oligonucleotide complexes made using peptides R11 and RG9 showed very similar distributions to complexes made using peptide R9. However, using peptides R7, K7, K9, K11, RG9, RRG and RRQ, either very few fluorescent complexes could be seen, or large apparent aggregates were detected. Light-mediated redistribution was seen with peptides R9, R11 and RG9, but not with R7, K7, K9, K11, RRG and RRQ.

These findings indicate in general that peptides containing arginine are better able to form complexes with fluorescently-labeled oligonucleotides than lysine-containing peptides. Without wishing to be bound by any particular theory, for peptides of the same size, the arginine content may be important, and replacing arginine residues with glycine interferes with peptide-oligonucleotide complex formation, perhaps by reducing the pI of the peptide.

### Example 5

### Delivery and Activation of Proteins

5.1. Materials and Methods

A fusion protein comprising VP22 and Cre recombinase was constructed using the vector pCRT7/VP22-1-TOPO (Invitrogen) essentially according to the product manual. The fusion protein comprises amino acids 159 to 301 of VP22 and amino acids 1 to 343 of Cre recombinase. The VP22/Cre recombinase fusion protein was expressed in E. coli and purified using the Voyager™ Protein production Kit 1 (Invitrogen).

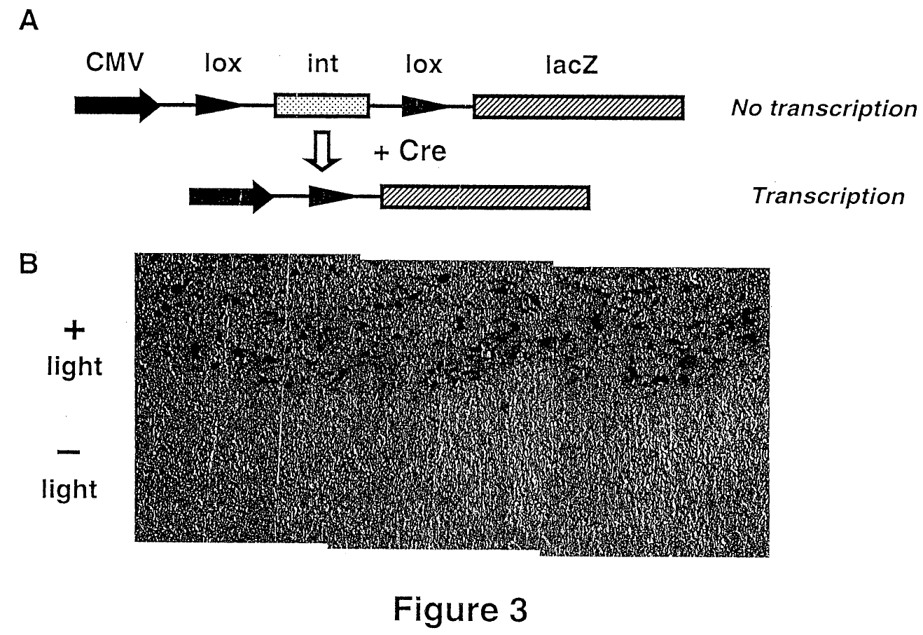

The fusion protein was tested using 293 cells transiently transfected with a Cre dependent lacZ reporter gene (FIG. 3A). In the absence of Cre, lacZ expression was not detected in any cell due to the insertion of a transcriptional termination cassette (Lasko, M., et al., Proc. Natl Acad. Sci. USA. 89:6232-6236 (1992)) between the CMV promoter and the lacZ ORF.

VP22-Cre was diluted to a concentration of 1 μM in 10 μl PBS (Invitrogen). The 5′-FITC-labeled oligonucleotide (FITC-anti-raf oligonucleotide) described in the preceding Examples was diluted to 0.5 μM in 10 ul PBS. The fusion protein-oligonucleotide mix was incubated for 10 min at room temperature. The medium in each well of cells was replaced with 0.5 ml DMEM/10% fetal bovine serum (Invitrogen). The protein-oligonucleotide complex was added to the medium, mixed and allowed to incubate with the cells for 16 hr at 37° C.

Prior to illumination, a boundary was set up between illuminated and unilluminated cells by covering half of the well with aluminum foil. Next, the medium was replaced and cells were observed using a Nikon fluorescence microscope equipped with FITC filter and 40× objective as above. The VP22-Cre fusion protein forms complexes with the FITC-oligonucleotide, and these complexes appear to have similar characteristics as the R9, Ant(42-58) and PTD3-FITC oligonucleotide complexes.

The VP22-Cre/FITC-oligonucleotide complexes were then treated by illumination for 10 min under the fluorescence microscope as before except that the objective was removed and light was allowed to reach the cells directly (without focusing). Dispersal of the VP22-Cre/FITC-oligonucleotide complexes was then seen in cells throughout the illuminated half of the well.

5.2. Results

After 40 hr, the cells were stained for β-galactosidase activity. A distinct boundary between illuminated and unilluminated cell could be seen. Cells having functional Cre recombinase activity, measured as cells expressing beta-galactosidase, were confined to the illuminated half of the well (FIG. 3B). In this system Cre recombinase activity, and hence lacZ expression, is controlled in a light dependent fashion.

These results indicate that activity of Cre recombinase may be controlled by addition of a short arginine containing tag thereto, i.e., as in a R9-Cre recombinase fusion protein. The Cre recombinase activity can be sequestered in complexes with a fluorescently labeled oligonucleotide (any oligonucleotde or other nucleic acid that would function for complex formation) until cells are illuminated. Furthermore, the addition of such a tag can be used in a general way to precisely control the activity of a protein. This can be especially useful for the manipulation of proteins involved in cell signaling events, where protein activation can occur in a few minutes following cell stimulation.

### Example 6

### Delivery and Activation of Short Interfering RNA (siRNA)

6.1. Materials and Methods

A stable 293 cell line expressing the luciferase gene was constructed using the Flp-In system according to the manufacturer's instructions (Invitrogen). A 21 bp siRNA directed against nucleotides 153-173 of the Photinus pyralis luciferase (GL2 variant) open reading frame was used. This siRNA has the sequence 5′-CGUACGCGGAAUACUUCGA-3′ (SEQ ID NO.:17) (E1bashir, S. M., et al., Nature 411:494-498 (2001)). A 21 bp siRNA directed against GFP was used to control for non-specific effects of siRNA delivery on gene expression (5′-CACUUGUCACUACUUUCUC-3′) (SEQ ID NO.:18). Each siRNA has an additional 3′ TdT overhang and was obtained from Xeragon, Inc. (Germantown, Md. 20874).

Prior to treatment, cells were grown in DMEM medium plus 10% FBS to 60-80% confluence in 24 well plates (0.5 ml medium/well). For each well of cells 100 pmol siRNA was diluted in 100 μl optiMEM medium (Invitrogen). 2 μl Lipofectamine 2000 was diluted separately in 100 μl optiMEM medium. After 5 min the diluted Lipofectamine 2000 and siRNA were combined and incubated at room temperature for 30 min before application to cells.

One hundred (100) pmol of each siRNA was also combined with 200 pmol FITC-R9 peptide. The FITC-R9 used in this experiment was a mixture of 5 and 6 FITC N terminal labeled R9. Application of complexes to cells and illumination was performed as described for VP22-Cre delivery and activation in the preceding Examples, with the exception that two separate plates of cells were used rather than one plate with an illuminated and non-illuminated half. One plate was kept in a 37° C. incubator without illumination. The second plate was treated as above and returned to the incubator for 24 hr. Cell lysates were then prepared and luciferase activity was measured using a EG+G Berthold microplate luminometer LB96V.

6.2. Results

As shown in Table 6 (below), light dependent reduction in luciferase expression was seen using the luciferase siRNA. The luciferase activity was similar to that seen when the siRNA was delivered using Lipofectamine 2000.

TABLE 6
Light Dependent Activation of Luciferase siRNA
and Inhibition of Luciferase Activity

| | With illumination | No illumination |
|---|---|---|
| | (RLU*) | (RLU*) |
| FITC-R9 + luciferase siRNA | 39418 | 218186 |
| FITC R9 + GFP siRNA | 182960 | 196455 |
| Lipofectamine 2000 + luciferase | 12919 | 11864 |
| siRNA | | |
| Lipofectamine 2000 + GFP | 228879 | 176527 |
| siRNA | | |
| Untreated cells | 228067 | n.d. |

*Relative luminescence units

### Example 7

### Delivery and Activation of Short Interfering RNA (siRNA)—Comparison of Different Fluorescently labeled Peptides

Experiments were performed as described in Example 6 except that a stable BHK cell line expressing the luciferase gene, constructed using the Flp-In system according to the manufacturer's instructions (Invitrogen), was used. Fluorescently-labeled R9 peptides were evaluated for their ability to deliver a siRNA against a luciferase reporter gene, leading to light-dependent knockdown of luciferase activity. The effect of certain labeling variations of the R9 peptide were assessed. Four different fluorescein-labeling reagents were employed to generate four different N-terminally labeled R9 peptides: 5-fluorescein succinimidyl ester (5-FAM), 6-fluorescein succinimidyl ester (6-FAM), 5-fluorescein isothiocyanate (5-FITC) and 6-fluorescein isothiocyanate (6-FTIC) as illustrated in the following structures:

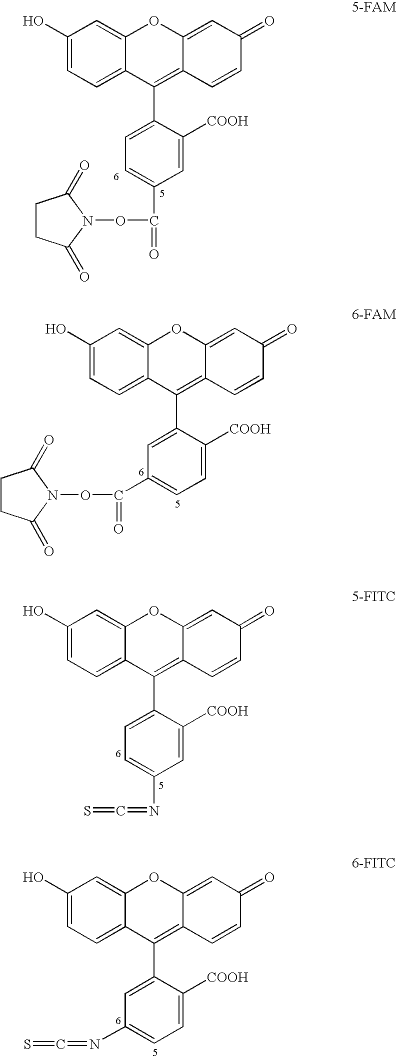

Fluoresecently labeled R9 peptides were obtained from Molecular Probes (prepared by custom synthesis). However, the N-terminal labeled R9 peptides can be prepared using known methods and commercially available reagents or labeling kits.

In the FITC labeled R9 peptides, the R9 peptide is linked through a thiourea linkage and in the FAM labeled R9 peptides, the R9 peptide is linked through a carboxyamide linkage:

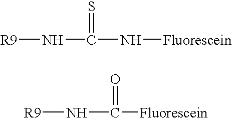

As in Example 6, 100 pmol of each siRNA was combined with 200 pmol of each of the fluorescein-labeled-R9 peptides. Application of complexes to cells and illumination was performed as described for VP22-Cre delivery and activation in the preceding Examples, with the exception that two separate plates of cells were used rather than one plate with an illuminated and non-illuminated half. One plate was kept in a 37° C. incubator without illumination. The second plate was treated as above and returned to the incubator for 24 hr. Cell lysates were then prepared and luciferase activity was measured using a EG+G Berthold microplate luminometer LB96V. Average luciferase reporter activity in treated cells after illumination is reported in Table 7.

TABLE 7
Average Luciferase Reporter Activity After Irradiation
(Total Relative Luciferase Activity, TRLA)*

| TREATMENT | TRLA |
|---|---|
| Untreated Cells | 837 |
| 6-FTIC | 673 |
| 5-FTIC | 609 |
| 6-FAM | 387 |
| 5-FAM | 86 |

*Delivering 10 pmol luciferase GL2 siRNA

These results indicate that the linkage between the peptide and the fluorescent molecule can affect the efficiency of delivery and/or release of a nucleotide delivered into cells using fluorescently-labeled translocating peptides.

### Example 8

### Delivery of Plasmid DNA

To test whether complexes between FITC-R9 peptides and plasmid DNA could be delivered to A-549 cells and dispersed in a light dependent fashion, the following experiment was performed with the plasmid pCMV●SPORT-βgal (Invitrogen). This plasmid can be used as a reporter vector to monitor transfection efficiency. The plasmid contains the E. coli β-galactosidase (β-gal) gene, a CMV promoter for high expression of β-gal in mammalian cells, and an SV40 polyadenylation signal downstream of the β-gal gene that direct proper processing of the mRNA in eukaryotic cells.

A range of plasmid amounts, from 0.5 μg to 3.75 ng, was mixed with 5 pmol of FITC-R9 peptide and applied to cells. Intracellular complexes were seen only when 12.5 ng or 6.25 ng plasmid were used. Prior to illumination, 5% of the cells comprised complexes. After photoillumination, nearly all of the complexes were redistributed.

These results demonstrate that complexes of FITC-R9 peptides and plasmids can be formed and dissociate following photoillumination.

### Example 9

### Chemically Mediated Redistribution of Oligonucleotides

CHO cells were treated with complexes containing the FITC-R9 peptide and the raf control oligonucleotide as described in the preceding Examples, except that chloroquine was included in the medium at a final concentration of 100 uM. After incubation for 16 hr at 37° C., cells were visualized using fluorescence microscopy. In approximately 50% of cells, fluorescence was seen distributed uniformly within the cells. The distribution in each cell appeared similar to that seen in experiments in which redistribution was seen as a result of photoillumination.

### Example 10

### Transfection Agents and Short Interfering RNA (siRNA)

The non-limiting examples of transfection agents described in Table 4 can be used in combination with the cellular delivery molecules and complexes described in the preceding Examples. These agents can also be used by themselves to deliver RNAi molecules. For example, Lipofectamine™ 2000 has been used to transfect siRNA into mammalian cells (Gitlin et al, Nature 418:379-380, 2002; Yu et al., Proc Natl Acad Sci USA 99:6047-6052, 2002), and Oligofectamine™ has been used to transfect siRNA into HeLa cells (E1bashir et al., Nature 411:494-498, 2001; Harborth et al., J Cell Sci 114:4557-4565, 2001).

In general, the following guidelines should be followed when using these transfection agents to introduce siRNA into cells. First, the cells should be transfected when they are about 30 to about 50% confluent. Second, antibiotics should not be added during the transfection as this may cause cell death. Third, for optimal results, the transfection agent should be diluted in Opti-MEM® I Reduced Media (Invitrogen) prior to being combined with siRNA.

### Example 11

### Multiwell Format

The compounds, compositions and methods described herein can be used to transfect cells in a multiwell format, e.g., a 24-, 48-, 96-, or 384-well plate. The following procedures describes the transfection of siRNA into cells using Lipofectamine™ 2000 or Oligofectamine™, and can be adapted to use with any other nucleic acids or transfection agents or combinations thereof.

In any procedure, one should have the following materials prepared beforehand: siRNA of interest (20 pmol/ul); prewarmed Opti-MEM® I Reduced Media (Invitrogen); and 24-well tissue culture plates and other tissue culture supplies. The cells to be transfected should be about 30 to about 50% confluent, and cell populations are preferably determined before transfection to comprise at least about 90% viable cells.

The following procedures are used to transfect mammalian cells in a 24-well format. To transfect cells in other tissue culture formats, optimal conditions for those formats might vary from those given herein for the 24-well format.

10.1 Lipofectamine™ 2000

For transfecting HEK293, BHK, CHO-1, or A549 cells, see Table 8 for suggested transfection conditions. Typically, in RNAi studies using these conditions, a decrease of ≧50%, preferably ≧70%, more preferably ≧80%, and most preferably ≧95% in the expression of a stably integrated reporter gene or an endogenous gene is observed by about 24 to about 48 hours after transfection.

TABLE 7
siRNA Transfection Conditions for Cell Lines

| | Cell Density | Amount of | Amount of |
|---|---|---|---|
| Cell Line | (cells/well) | Lipofectamine ™ 2000 | siRNA |
| HEK 293 | 1 × 10⁵ | 1 μl | 20 pmol |
| BHK | 1.5 × 10⁴ | 1 μl | 20 pmol |
| CHO-K1 | 4 × 10⁴ | 1 μl | 20 pmol |
| A549 | 1.5 × 10⁴ | 1 μl | 20 pmol |

1. One day before transfection, plate cells in 0.5 ml of growth medium without antibiotics so that they will be about 30 to about 50% confluent at the time of transfection.

2. For each transfection sample, prepare siRNA:Lipofectamine™ 2000 complexes as follows:

(a) Dilute the appropriate amount of siRNA in 50ml of Opti-MEM® Reduced Serum Medium without serum (or other medium without serum). Mix gently.

(b) Mix Lipofectamine™ 2000 gently before use, then dilute the appropriate amount in 50 ul of Opti-MEM®M Medium (or other medium without serum). Mix gently and incubate for 5 minutes at room temperature. Note: Combine the diluted Lipofectamine™ 2000 with the diluted siRNA within 30 minutes. Longer incubation times may decrease activity. If D-MEM is used as a diluent for the Lipofectamine™ 2000, mix with the diluted siRNA within 5 minutes.

(c) After the 5 minute incubation, combine the diluted siRNA with the diluted Lipofectamine™ 2000 (total volume is 100 ml). Mix gently and incubate for 20 minutes at room temperature.

3. Add the 100 ml of the siRNA/Lipofectamine™ 2000 mixture to each well. Mix gently by, for example, rocking the plate back and forth.

4. Incubate the cells at 37° C. in a CO₂ incubator for about 24 to about 72 hours until they are ready to be assayed for gene expression. It is generally not necessary to remove the complexes or change the medium; however, growth medium may be replaced after about 4 to about 6 hours without loss of tranfection activity.

10.2 Oligofectamine™

Typically, in RNAi studies of HeLa cells using the following conditions, a decrease of ≧50%, preferably ≧70%, more preferably ≧80%, and most preferably ≧95% in the expression of a stably integrated reporter gene or an endogenous gene is observed by about 24 to about 48 hours after transfection.

1. One day before transfection, plate cells in 0.5 ml of growth medium without antibiotics so that they will be about 50% confluent at the time of transfection.

2. For each transfection sample, prepare siRNA:Oligofectamine™ complexes as follows:

(a) Dilute 60 pmol of siRNA in 50 ul of Opti-MEM® Reduced Serum Medium without serum (or other medium without serum). Mix gently.

(b) Mix Oligofectamine™ gently before use, then dilute 3 ul in 12 ul of Opti-MEM® Medium (or other medium without serum). Mix gently and incubate for 5 minutes at room temperature.

(c) After the 5 minute incubation, combine the diluted siRNA with the diluted Oligofectamine™ (total volume is 68 ul). Mix gently and incubate for 20 minutes at room temperature.

3. Add the 68 ul of the siRNA:Oligofectamine™ mixture to each well. Mix gently by, for example, rocking the plate back and forth.

4. Incubate the cells at 37° C. in a CO₂ incubator for about 24 to about 72 hours until they are ready to be assayed for gene expression. It is generally not necessary to remove the complexes or change the medium; however, growth medium may be replaced after about 4 to about 6 hours without loss of tranfection activity.

One skilled in the art will readily appreciate that the present invention is well adapted to carry out the objects and obtain the ends and advantages described, as well as those inherent therein. The compositions and transportable complexes and the methods, procedures, treatments, molecules, and specific compounds described herein are presently representative of certain aspects of the invention, and thus are exemplary and are not intended as limitations on the scope of the invention. Alternatives, equivalents, changes, and other uses will occur to those skilled in the art, and those are encompassed within the spirit of the invention are defined by the scope of the claims below. It will be readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. Thus, for example, in each instance herein any of the terms “comprising,” “consisting essentially of,” and “consisting of” may be replaced with either of the other two terms. The terms and expressions that have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed herein, optional features, modification and variation of the concepts herein disclosed may be resorted to by those skilled in the art, and that such modifications and variations are considered to be within the scope of this invention as defined by the appended claims. In addition, where features or aspects of the invention are described in terms of Markush groups, those skilled in the art will recognize that the invention is also thereby described in terms of any individual member or subgroup of members of the Markush group.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other aspects of the invention are within the following claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference. References cited herein are incorporated by reference herein, at least in part, to provide details of various techniques (e.g., synthetic methods and assays methods), and sources of materials, (e.g., translocating peptides, biologically active molecules or other complex components).

What is claimed is:

1. A method of delivering a polypeptide to a cell, comprising:
(a) contacting said cell with, in any order or combination, said polypeptide, a nucleic acid, a fluorescent molecule, and a cellular delivery molecule; and
(b) treating said cell with a treatment that results in the dissociation of said polypeptide from one or more of said nucleic acid, said fluorescent molecule, and said cellular delivery molecule.

2. The method of claim 1, wherein said treatment comprises irradiation.

3. The method of claim 1, wherein two or more of said polypeptide, said nucleic acid, said fluorescent molecule, and said cellular delivery molecule are admixed before said contacting.

4. The method of claim 1, wherein said cellular delivery molecule is a cellular delivery polypeptide.

5. The method of claim 4, wherein said cellular delivery polypeptide is a synthetic peptide.

6. The method of claim 4, wherein said polypeptide is comprised within a fusion protein that further comprises said cellular delivery polypeptide.

7. The method of claim 6, wherein said fusion protein further comprises an accessory polypeptide.

8. The method of claim 7, wherein said accessory polypeptide is an enzyme that has a nucleic acid as one of its reactants or one of its products.

9. The method of claim 8, wherein said accessory polypeptide is a recombinase.

10. The method of claim 9, wherein said recombinase is a site-specific recombinase.

11. The method of claim 9, wherein said nucleic acid comprises at least one site recognized by said site-specific recombinase.

12. The method of claim 4, wherein said cellular delivery polypeptide:
(a) comprises m % basic amino acids, wherein m % is from about 50% to 100%;
(b) comprises a sequence of n contiguous basic amino acids, wherein n is any whole integer between 2 and about 75;
and, additionally or alternatively,
(c) has an amino acid sequence that is not present in the amino acid sequence of a protein encoded by herpes simplex virus (HSV).

13. The method of claim 12, wherein said polypeptide is a recombinase.

14. The method of claim 13, wherein said recombinase is a site-specific recombinase.

15. The method of claim 14, wherein said nucleic acid comprises at least one site recognized by said site-specific recombinase.

16. The method of claim 4, wherein said cellular delivery polypeptide has a pI of from about 10.5 to about 14.

17. The method of claim 12, wherein an oligopeptide having the sequence of said n contiguous basic amino acids has a pI of from about 10.5 to about 14.

18. The method of claim 1, wherein said fluorescent molecule is a fluorescent polypeptide.

19. The method of claim 18, wherein said polypeptide is comprised within a fusion protein that further comprises said fluorescent polypeptide.

20. The method of claim 4, wherein said fluorescent molecule is a fluorescent polypeptide.

21. The method of claim 20, wherein said fluorescent polypeptide is comprised within a fusion protein that further comprises said polypeptide and, additionally or alternatively, said fluorescent polypeptide.

22. The method of claim 20, wherein one or more of said polypeptide, said cellular delivery polypeptide and said fluorescent polypeptide are comprised within a fusion protein that further comprises an accessory polypeptide.

23. The method of claim 22, wherein said accessory polypeptide is an enzyme that has a nucleic acid as one of its reactants or one of its products.

24. The method of claim 1, wherein said cellular delivery molecule is a nucleic acid binding protein.

25. The method of claim 24, wherein said nucleic acid binding protein is selected from the group consisting of a histone, a histonelike protein, and poly-Lysine, and combinations and derivatives thereof.

26. The method of claim 4, wherein two or more of said polypeptide, said nucleic acid, said fluorescent molecule, and said cellular delivery polypeptide are admixed before said contacting.

27. The method of claim 1, wherein said nucleic acid is an oligonucleotide.

28. A method of delivering a polypeptide to a cell, comprising:
(a) contacting said cell with, in any order or combination, said polypeptide, a nucleic acid, a fluorescent molecule, a cellular delivery molecule, and a transfection agent; and
(b) treating said cell with a treatment that results in the dissociation of said polypeptide from one or more of said nucleic acid, said fluorescent molecule, and said cellular delivery molecule.

29. The method of claim 28, wherein two or more of said polypeptide, said nucleic acid, said fluorescent molecule, and said cellular delivery molecule are admixed before said contacting.

30. The method of claim 28, wherein one or more of said fluorescent molecule and said cellular delivery molecule is a polypeptide.

31. A kit comprising at least one fluorescent molecule and at least one cellular delivery molecule.

32. The kit of claim 31, further comprising one or more elements selected from the group consisting of one or more transfection agents, one or more cells, one or more nucleic acids, one or more sets of instructions, and one or more photoilluminators and, optionally, a power supply therefor.

33. The kit of claim 31, wherein one or both of said cellular delivery molecule and said fluorescent molecule are polypeptides.

34. The kit of claim 33, wherein said cellular delivery polypeptide and said fluorescent polypeptide are comprised within a fusion protein.

35. The kit of claim 31, further comprising at least one RNAi molecule.

36. The kit of claim 31, further comprising one or more cells.

37. The kit of claim 36, wherein said cells are competent for transfection or transformation.

38. The kit of claim 36, wherein said cells express or overexpress dicer.

39. A kit comprising at least one transfection agent and at least one RNAi molecule.

40. The kit of claim 39, further comprising one or more elements selected from the group consisting of one or more cells, one or more recombinases, one or more recombination proteins, and one or more sets of instructions.

41. A complex comprising a cellular delivery polypeptide and an agent that is desirably taken up by cells, wherein said cellular delivery polypeptide comprises a fluorescent moiety.

42. The complex of claim 41, wherein the activity of said agent that is desirably taken up by cells is repressed within said complex.

43. The complex of claim 42, wherein said agent is activated once said agent dissociates from said complex.

44. The complex of claim 41, wherein the dissociation of said cellular delivery polypeptide from said complex is photoactivatable.

45. The complex of claim 41, wherein the dissociation of said agent that is desirably taken up by cells from said complex is photoactivatable.

46. The complex of claim 41, further comprising nucleic acid.

47. The complex of claim 41, wherein said nucleic acid comprises from about 5 bases to about 200 kilobases.

48. The complex of claim 46, wherein said nucleic acid is selected from the group consisting of mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof.

49. The complex of claim 46, wherein said nucleic acid is an oligonucleotide.

50. The complex of claim 46, wherein said nucleic acid comprises one or more chemical modifications.

51. The complex of claim 46, wherein said nucleic acid is said agent that is desirably taken up by cells.

52. The complex of claim 46, wherein said cellular delivery polypeptide is comprised within a fusion protein with an accessory polypeptide.

53. The complex of claim 52, wherein said accessory polypeptide is biologically active.

54. The complex of claim 53, wherein said accessory polypeptide is selected from the group consisting of an affinity tag, an epitope, a protease cleavage site, a detectable polypeptide, an enzyme, a hormone, a receptor ligand, a receptor fragment, and an antibody or antibody derivative.

55. The complex of claim 41, wherein said fluorescent moiety is a fluorescent polypeptide.

56. The complex of claim 55, wherein said fluorescent polypeptide is comprised within a fusion protein with a second polypeptide.

57. The complex of claim 52, wherein said accessory polypeptide is said cellular delivery polypeptide.

58. The complex of claim 41, further comprising one or more transfection agents.

59. The complex of claim 41, further comprising one or more recombinases and, additionally or alternatively, a recombination protein.

60. A kit comprising the complex of claim 59.

61. A method of delivering a nucleic acid to a cell, comprising:
(a) contacting said cell with, in any order or combination, said nucleic acid, a fluorescent molecule, and a cellular delivery molecule; and
(b) treating said cell with a treatment that results in the dissociation of said nucleic acid from one or both of said fluorescent molecule and said cellular delivery molecule.

62. The method of claim 61, wherein said treatment comprises irradiation.

63. The method of claim 61, wherein said nucleic acid is biologically active following said treatment.

64. The method of claim 61, wherein said nucleic acid is dispersed in the cytoplasm of said cell following said treatment.

65. The method of claim 61, wherein said nucleic acid comprises from about 5 bases to about 200 kilobases.

66. The method of claim 61, wherein said nucleic acid is selected from the group consisting of mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof.

67. The method of claim 61, wherein said nucleic acid is an oligonucleotide.

68. The method of claim 61, wherein said nucleic acid comprises one or more chemical modifications.

69. The method of claim 61, wherein said fluorescent molecule is not attached to said nucleic acid.

70. The method of claim 61, wherein said fluorescent molecule is attached to said cellular delivery molecule.

71. The method of claim 61, wherein said cellular delivery molecule is a cellular delivery polypeptide.

72. The method of claim 71, wherein said cellular delivery polypeptide is a synthetic peptide.

73. The method of claim 71, wherein said cellular delivery polypeptide:
(a) comprises m % basic amino acids, wherein m % is from about 50% to 100%;
(b) comprises a sequence of n contiguous basic amino acids, wherein n is any whole integer between 2 and about 75;
and, additionally or alternatively,
(c) has an amino acid sequence that is not present in the amino acid sequence of a protein encoded by herpes simplex virus (HSV).

74. The method of claim 71, wherein said said cellular delivery polypeptide is comprised within a fusion protein.

75. The method of claim 74, wherein said fusion protein further comprises an accessory polypeptide.

76. The method of claim 75, wherein said accessory polypeptide is an enzyme that has a nucleic acid as one of its reactants or one of its products.

77. The method of claim 76, wherein said accessory polypeptide is a recombinase.

78. The method of claim 77, wherein said recombinase is a site-specific recombinase.

79. The method of claim 78, wherein said nucleic acid comprises at least one site recognized by said site-specific recombinase.

80. The method of claim 61, wherein said nucleic acid includes a sequence that encodes a protein or a portion thereof.

81. The method of claim 80, wherein said sequence encodes an amino acid sequence of a portion of a protein in said cell, wherein a cellular nucleic acid encoding said protein, or a portion thereof, is desirably replaced by said sequence.

82. The method of claim 80, wherein said protein is expressed in said cell.

83. The method of claim 73, wherein said cellular delivery polypeptide has a pI of from about 10.5 to about 14.

84. The method of claim 73, wherein an oligopeptide having the sequence of said n contiguous basic amino acids has a pI of from about 10.5 to about 14.

85. The method of claim 61, wherein said fluorescent molecule is a fluorescent polypeptide.

86. The method of claim 71, wherein said fluorescent molecule is a fluorescent polypeptide.

87. The method of claim 86, wherein said fluorescent molecule and said cellular delivery molecule are polypeptides that are comprised within a fusion protein.

88. A method of delivering a nucleic acid to a cell, comprising:
(a) contacting said cell with, in any order or combination, said nucleic acid, a fluorescent molecule, a cellular delivery molecule, and a transfection agent; and
(b) treating said cell with a treatment that results in the dissociation of said nucleic acid from one or more of said fluorescent molecule and said cellular delivery molecule.

89. The method of claim 88, wherein two or more of said nucleic acid, said fluorescent molecule, and said cellular delivery molecule are admixed before said contacting.

90. The method of claim 88, wherein one or more of said fluorescent molecule and said cellular delivery molecule is a polypeptide.

91. A molecular complex comprising one or more nucleic acids, one or more fluorophores, and one or more cellular delivery polypeptides, wherein each cellular delivery polypeptide:
(a) is m % basic amino acids, wherein m is from about 10% to 100%;
(b) comprises a sequence of n contiguous basic amino acids, wherein n is any whole integer between 2 and 50; and
(c) is not derived from a herpes simplex virus (HSV) protein.

92. The molecular complex of claim 91, wherein said fluorphore is covalently linked to either said nucleic acid or said cellular delivery polypeptide.

93. A composition comprising the molecular complex of claim 91.

94. The composition of claim 93 further comprising one or more agents selected from the group consisting of a transfection agent, a transfection enhancing agent, and an endosome disrupting agent.

95. A cell comprising the molecular complex of claim 91.

96. A composition comprising the cell of claim 95.

97. The composition of claim 96, wherein said cell remains viable after said composition is frozen.

98. The composition of claim 97, wherein said composition comprises glycerol.

99. A container comprising the molecular complex of claim 91.

100. A pharmaceutical composition comprising the molecular complex of claim 91 and a pharmaceutically acceptable excipient or carrier.

101. The pharmaceutical composition of claim 100, wherein one or more of said nucleic acid, said polypeptide and said fluorophore is biologically active.

102. The pharmaceutical composition of claim 100, wherein said nucleic acid is biologically active.

103. The pharmaceutical composition of claim 102, wherein said biologically active nucleic acid is selected from the group consisting of mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof.

104. The pharmaceutical composition of claim 100, wherein said polypeptide is biologically active.

105. A method of treating an individual suffering from a disease or disorder, said method comprising contacting said individual with the complex of claim 91, the composition of claim 93, or the pharmaceutical composition of claim 100.

106. The method of claim 105, further comprising exposing said individual to electromagnetic radiation.

107. A method of providing gene therapy to an individual in need thereof, comprising contacting said individual, or cells therefrom, with the complex of claim 91, the composition of claim 93, or the pharmaceutical composition of claim 100.

108. A method of determining a cellular response to a test compound comprising:
(a) contacting a first cell with, in any order or combination, a first nucleic acid, a fluorescent molecule, and a cellular delivery molecule;
(b) contacting a second cell with, in any order or combination, a second nucleic acid, said fluorescent molecule, and said cellular delivery molecule;
(c) treating said cells with a treatment that results in the dissociation of said polypeptide from one or more of said nucleic acid, said fluorescent molecule, and said cellular delivery molecule;
(d) contacting said cells with said test compound, before (a); during (a), (b) or (c); between (a) and (b); between (b) and (c); and, additionally or alternatively, after (c);
(e) detecting a signal from said cells, wherein said signal corresponds to a cellular response; and
(f) comparing the signal from said first cell with the signal from said second cell.

109. The method of claim 108, wherein one or more of said cells comprises one or more reporter genes.

110. The method of claim 108, wherein one or more of said nucleic acid, said cellular delivery molecule and said fluorophore is biologically active.

111. The method of claim 108, wherein said nucleic acid is biologically active.

112. The method of claim 111, wherein said biologically active nucleic acid is selected from the group consisting of mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof.

113. A method of identifying a compound having a preselected activity or effect comprising:
(a) contacting a first cell with, in any order or combination, a first nucleic acid, a fluorescent molecule, and a cellular delivery molecule;
(b) contacting a second cell with, in any order or combination, a second nucleic acid, a fluorescent molecule, and a cellular delivery molecule;
(c) treating said cells with a treatment that results in the dissociation of said polypeptide from one or more of said nucleic acid, said fluorescent molecule, and said cellular delivery molecule;
(d) contacting said cells with said test compound, before (a); during (a), (b) or (c); between (a) and (b); between (b) and (c); and, additionally or alternatively, after (c);
(e) detecting a signal from said cells, wherein said signal corresponds to a cellular response; and
(f) comparing the signal from said first cell with the signal from said second cell,
wherein a difference in the signal from first cell from the signal from said second cell corresponds to said preselected activity or effect.

114. The method of claim 113, wherein one or more of said cells comprise one or more reporter genes.

115. The method of claim 113, wherein one or more of said nucleic acid, said cellular delivery molecule and said fluorophore is biologically active.

116. The method of claim 113, wherein said nucleic acid is biologically active.

117. The method of claim 116, wherein said biologically active nucleic acid is selected from the group consisting of mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof.
